(19) United States
(12) Patent Application Publication
(10) Pub. No.: US 2024/0245786 A1
(43) Pub. Date: Jul. 25, 2024

(54) SMALL MOLECULE DEGRADERS OF PHOSPHATIDYLINOSITOL-5-PHOSPHATE 4-KINASE TYPE 2 AND USES THEREOF

(71) Applicants: DANA-FARBER CANCER INSTITUTE, INC., Boston, MA (US); CORNELL UNIVERSITY, Ithaca, NY (US)

(72) Inventors: Nathanael S. Gray, Boston, MA (US); Tinghu Zhang, Brookline, MA (US); Lewis C. Cantley, Cambridge, MA (US); Sindhu Carmen Sivakumaren, Boston, MA (US); Theresa Manz, Somerville, MA (US); Eric Wang, Brookline, MA (US); Wenzhi Ji, Boston, MA (US); Eric S. Fischer, Chestnut Hill, MA (US); Katherine Donovan, Boston, MA (US)

(73) Assignees: DANA-FARBER CANCER INSTITUTE, INC., Boston, MA (US); CORNELL UNIVERSITY, Ithaca, NY (US)

(21) Appl. No.: 18/269,122

(22) PCT Filed: Dec. 22, 2021
(86) PCT No.: PCT/US2021/064880

§ 371 Date: Jun. 22, 2023

Related U.S. Application Data
(60) Provisional application No. 63/130,150, filed on Dec. 23, 2020.
(60) Provisional application No. 63/273,388, filed on Oct. 29, 2021.

(51) Int. Cl. A61K 47/55; A61K 31/506; C07D 401/14; C07D 417/14

(52) U.S. Cl. CPC A61K 47/55; A61K 31/506; C07D 401/14; C07D 417/14

(57) ABSTRACT

The present invention relates to bifunctional compounds, compositions, and methods for treating diseases or conditions by modulating (e.g., reducing) the level or activity of at least one of PIP4K2A, PIP4K2B, and PIP4K2C.

### RELATED APPLICATIONS

This application claims the benefit of priority under 35 U.S.C. § 119(e) to U.S. Provisional Application No. 63/130,150, filed on Dec. 23, 2020 and to U.S. Provisional Application No. 63/273,388, filed on Oct. 29, 2021, each of which is incorporated herein by reference in its entirety.

### GOVERNMENT LICENSE RIGHTS

This invention was made with government support under grant number 5R01CA197329-05 awarded by the National Institutes of Health. The government has certain rights in the invention.

### BACKGROUND OF THE INVENTION

The phosphatidylinositol 5-phosphate 4-kinases (PI5P4Ks), consisting of the three isoforms, PI5P4Kα, β, and γ, are lipid kinases that catalyze phosphorylation of phosphatidylinositol 5-phosphate (PI5P) on its 4-position to form phosphatidylinositol-4,5-bisphosphate (PI-4,5-P2) (Rameh et al., Nature 390(6656): 192-196 (1997). In the cellular membrane, PI-4,5-P2 is also produced by another signaling pathway in which phosphatidylinositol 4-phosphate (PI4P) is phosphorylated by phosphatidylinositol 4-phosphate 5-kinases (PI4P5Ks). Although the majority of PI-4,5-P2 is produced via the PI4P5K pathway, the PI5P4Ks have been recognized as key regulators of many cell functions including metabolism, stress response, autophagy, and immunological processes (Hu et al., J. Lipid Res. 59:507-514 (2018); Lamia et al., Mol. Cell. Biol. 24(11):5080-5087 (2004); Shim et al., Proc. Natl. Acad. Sci. U.S.A. 113(27):7596-7601 (2016); Al-Ramahi et al., eLife 6:e29123 (2017); Lundquist et al., Mol. Cell 70(3):531-543 (2018); Bulley et al., Proc. Natl. Acad. Sci. U.S.A. 113(38):10571-10576 (2016); Keune et al., Adv. Biol. Regul. 53(2):179-189 (2013)). PIP4Ks have distinct catalytic and non-catalytic functions in controlling cellular metabolism and suppress PIP5K activity and insulin-stimulated production of PI(3,4,5)P3 (Wang et al., Cell Rep. 27:1991-2001 (2019)). Dysregulation of the PI5P4K signaling pathway has been further linked to diseases such as diabetes, neurodegenerative disorders, and cancers (Lamia et al., Mol. Cell. Biol. 24(11): 5080-5087 (2004); Al-Ramahi et al., eLife 6:e29123 (2017); Jude et al., Oncogene 34(10): 1253-1262 (2015); Luoh et al., Oncogene 23:1354-1363 (2004); Emerling et al., Cell 155(4):844-857 (2013). Analysis of PI(4,5)P2 levels in cells with single or double knockdown of PIP4K isoforms revealed an additive effect among all three isoforms that does not correlate with their relative catalytic activities. Double knockdown of the most active isoforms, PIP4KA/B, failed to phenocopy the triple knockdown, suggesting that catalytic activity is not the most important factor in regulating PI(4,5)P2 levels (Wang et al., Cell Rep. 27:1991-2001 (2019).

These findings indicate that the inhibition of PI5P4K kinase activity might have a therapeutic potential across various diseases.

### SUMMARY OF THE INVENTION

A first aspect of the present invention is directed to a bifunctional compound, comprising a targeting ligand that binds at least one of phosphatidylinositol-5-phosphate 4-kinase type 2 alpha (PIP4K2A), PIP4K2B, and PIP4K2C and a degron covalently attached to the targeting ligand by a linker, wherein the compound has a structure represented by formula (I):

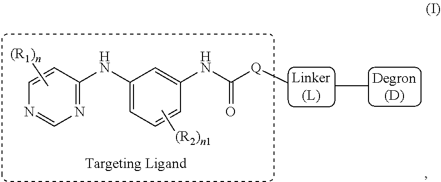

or a pharmaceutically acceptable salt or stereoisomer thereof, wherein:
   Q represents a bond or

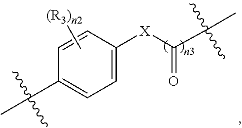

wherein X is a bond, CH₂, NH, or O;
each R₁ independently represents optionally substituted aryl, optionally substituted heteroaryl having 1-3 heteroatoms selected from N, O, and S, or NR₄R₅;
   each R₂ and R₃ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
   each R₄ and R₅ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, optionally substituted C₅-C₆ carbocyclyl or optionally substituted C₅-C₆ heterocarbocyclyl;
   n represents 1, 2, or 3;
   n₁ and n₂ independently represent 1, 2, 3, or 4;
   n₃ represents 0 or 1; and the
degron is a moiety that binds an E3 ubiquitin ligase.

Another aspect of the present invention is directed to a pharmaceutical composition containing a therapeutically effective amount of a bifunctional compound of formula (I) or a pharmaceutically acceptable salt or stereoisomer thereof, and a pharmaceutically acceptable carrier.

In another aspect of the present invention, methods of making the bifunctional compounds are provided.

A further aspect of the present invention is directed to a method of treating a disease or disorder by modulating (e.g., reducing) the level or activity of at least one of PIP4K2A, PIP4K2B, and PIP4K2C, that includes administering a therapeutically effective amount of a bifunctional compound of formula (I) or a pharmaceutically acceptable salt or stereoisomer thereof, to a subject in need thereof.

In some aspects, the present invention is directed to methods of modulating (e.g., enhancing) immune functions in a subject in need thereof, comprising administering to a subject a therapeutically effective amount of a bifunctional compound of formula (I) or pharmaceutically acceptable salt or stereoisomer thereof.

In some aspects, the present invention is directed to methods of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of a bifunctional compound of formula (I) or pharmaceutically acceptable salt or stereoisomer thereof.

As shown in working examples herein, bifunctional compounds embraced by formula (I) (also referred to herein as degraders) promote the degradation of at least one of PIP4K2A, PIP4K2B, and PIP4K2C.

Without intending to be bound by any particular theory of operation, the bispecific compounds of formula (I) are believed to cause degradation of at least one of PIP4K2A, PIP4K2B, and PIP4K2C by recruitment of cells' Ubiquitin/Proteasome System, whose function is to routinely identify and remove damaged proteins, into close proximity with PIP4K2A, PIP4K2B, or PIP4K2C as a result of binding between PIP4K2A, PIP4K2B, or PIP4K2C, and the targeting ligand. After destruction of a PIP4K2A, PIP4K2B, or PIP4K2C protein, the degrader is released and continues to be active. Thus, by engaging and exploiting the body's own natural protein disposal system, the bifunctional compounds of the present invention may represent a potential improvement over current small molecule inhibitors of PIP4K2A, PIP4K2B, and PIP4K2C and may overcome one or more limitations regarding their use. Therefore, effective intracellular concentrations of the degraders may be significantly lower than for small molecule PIP4K2A, PIP4K2B, or PIP4K2C inhibitors. Also, although PIP4K2A, PIP4K2B, and/or PIP4K2C genetic knockdown or knockout can be used to reduce the cellular concentration or amount of these proteins, it may be preferable to post-translationally disrupt, degrade, or destabilize PIP4K2A, PIP4K2B, and/or PIP4K2C proteins. Targeting proteins directly, rather than via the DNA or mRNA molecules that encode them, is a more direct and rapid method for reducing the scaffolding function of PIP4K proteins. Hence, degradation can allow some PIP4K2A, PIP4K2B, and/or PIP4K2C catalytic function to proceed while reducing the non-catalytic functions such as scaffolding between PIP4K2A, PIP4K2B, and/or PIP4K2C proteins and other cellular proteins and structures. Collectively, the present bifunctional compounds may represent a set of new chemical tools for at least one of PIP4K2A, PIP4K2B, and PIP4K2C knockdown and may provide a potential treatment modality for PIP4K2A, PIP4K2B, and/or PIP4K2C-associated cancers and autophagy-dependent diseases (e.g., neurodegenerative disorders, insulin). The bifunctional compounds may be used to enhance immune functions as described in WO2020210686 and Wang et al., Cell Rep. 27:1991-2001 (2019), each of which is incorporated by reference herein in its entirety.

### BRIEF DESCRIPTION OF THE DRAWINGS

FIG. 1A is an immunoblot of phosphatidylinositol-5-phosphate 4-kinase type 2 gamma (PIP4K2C) degradation in Molt4 cells treated with inventive bifunctional compounds 13-15 at the indicated concentrations for 8 h.

FIG. 1B is an immunoblot of PIP4K2C degradation in Molt4 cells treated with inventive bifunctional compound 14 at the indicated concentrations for 6 h.

FIG. 1C is a set of immunoblots of PIP4K2A, PIP4K2B, and PIP4K2C degradation in Molt4 cells treated with inventive bifunctional compound 1 at the indicated concentrations for 5 h or 8 h.

FIG. 1D is an immunoblot of PIP4K2A, PIP4K2B, PIP4K2C, and ERF degradation in Molt4 cells treated with inventive bifunctional compound 1 at the indicated concentrations for 6 h or 8 h.

FIG. 1E is an immunoblot of PIP4K2C degradation in Molt4 cells treated with inventive bifunctional compounds 1 and 8 at the indicated concentrations for 4h.

FIG. 1F is an immunoblot of PIP4K2C degradation in Molt4 cells treated with 1 μM of inventive bifunctional compounds 4 and 6-9 for 6 h.

FIG. 2 is a scatter plot that shows the relative change in relative protein abundance in Molt4 cells treated with 1 μM of inventive bifunctional compound 1 compared to dimethyl sulfoxide (DMSO) control.

FIG. 3A is an immunoblot of PIP4K2C degradation in Molt4 cells treated with inventive bifunctional compounds 1, 21, and 27-33 at the indicated concentrations for 4h.

FIG. 3B is an immunoblot of PIP4K2A, PIP4K2B, and PIP4K2C degradation in Molt4 cells treated with inventive bifunctional compounds 1, 8, 20, 21, and 25 at the indicated concentrations for 6 h.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C1), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein, which in this invention is at least one of phosphatidylinositol-5-phosphate 4-kinase type 2 alpha (PIP4K2A), PIP4K2B, and PIP4K2C, typically refers to an inter-molecular interaction that may be preferential (also referred to herein as “selective”) in that degradation of other proteins present in the cell is less and, in some cases, substantially less or even functionally insignificant.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bifunctional compounds comprise a targeting ligand that binds at least one of phosphatidylinositol-5-phosphate 4-kinase type 2 alpha (PIP4K2A), PIP4K2B, and PIP4K2C and a degron covalently attached to the targeting ligand by a linker, wherein the compound has a structure represented by formula (I):

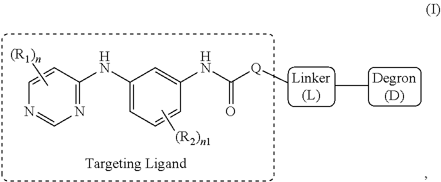

or a pharmaceutically acceptable salt or stereoisomer thereof, wherein:
   Q represents a bond or

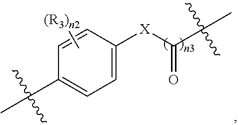

wherein X is a bond, CH₂, NH, or O;
each R₁ independently represents optionally substituted aryl, optionally substituted heteroaryl having 1-3 heteroatoms selected from N, O, and S, or NR₄R₅;
   each R₂ and R₃ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
   each R₄ and R₅ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, optionally substituted C₅-C₆ carbocyclyl or optionally substituted C₅-C₆ heterocarbocyclyl;
   n represents 1, 2, or 3;
   n₁ and n₂ independently represent 1, 2, 3, or 4;
   n₃ represents 0 or 1; and the
degron is a moiety that binds an E3 ubiquitin ligase.

In some embodiments, Q is a bond.

In some embodiments, Q is

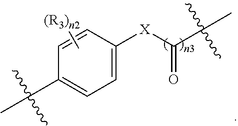

In some embodiments, wherein X is N.

In some embodiments, R₁ is optionally substituted aryl. In some embodiments, the optionally substituted aryl is optionally substituted phenyl.

In some embodiments, R₁ is

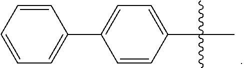 [SMILES: *c1ccc(-c2ccccc2)cc1]

In some embodiments, R₁ is optionally substituted heteroaryl having 1-3 heteroatoms selected from N, O, and S. In some embodiments, the optionally substituted heteroaryl is optionally substituted indole, indazole, or azaindole.

In some embodiments, R₁ is

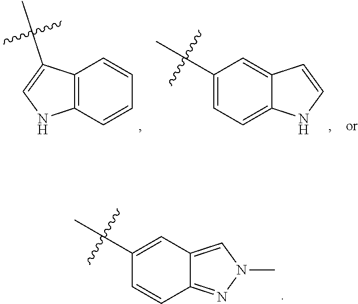 [SMILES: *c1c[nH]c2ccccc12 ; *c1ccc2[nH]ccc2c1 ; *c1ccc2nn(C)cc2c1]

In some embodiments, R₁ is NR₄R₅. In some embodiments, one of R₄ and R₅ is H. In some embodiments, one of R₄ and R₅ is optionally substituted C₅-C₆ carbocyclyl or optionally substituted C₅-C₆ heterocarbocyclyl.

In some embodiments, one of R₄ and R₅ is optionally substituted aryl. In some embodiments, the optionally substituted aryl is optionally substituted phenyl.

In some embodiments, R₁ is

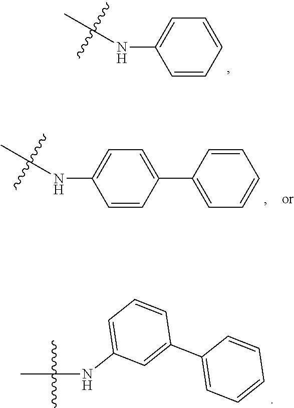 [SMILES: *Nc1ccccc1 ; *Nc1ccc(-c2ccccc2)cc1 ; *Nc1cccc(-c2ccccc2)c1]

In some embodiments, n is 1.

In some embodiments, R₂ and R₃ are H.

In some embodiments, the targeting ligand is represented by any one of structures TL1-TL7:

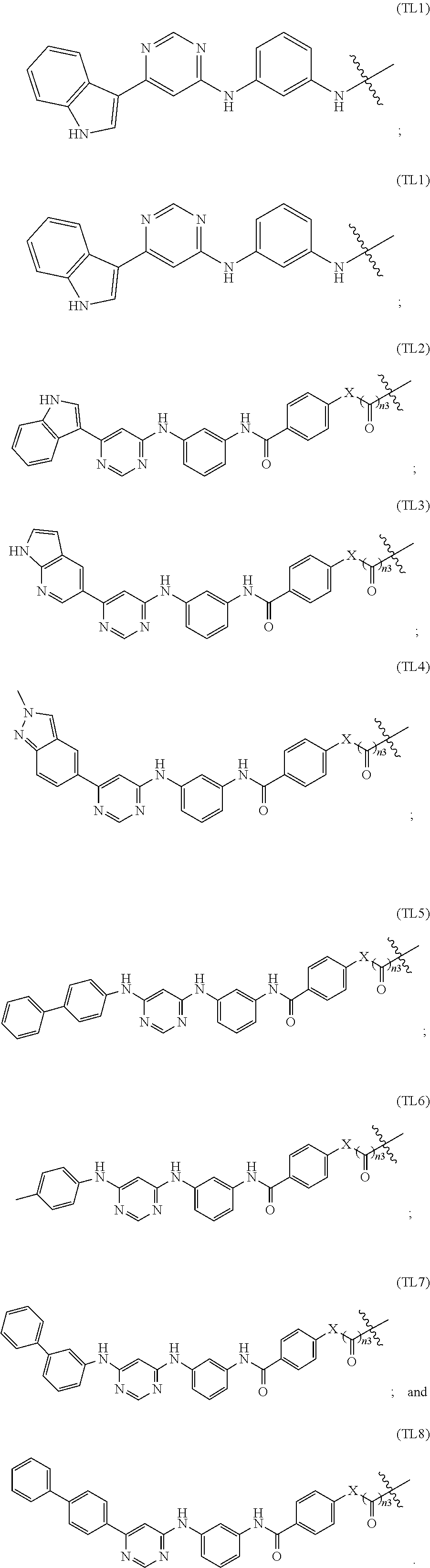

Thus, in some embodiments, the bifunctional compounds of the present invention may be represented by any one of the structures (I-1) to (I-8):

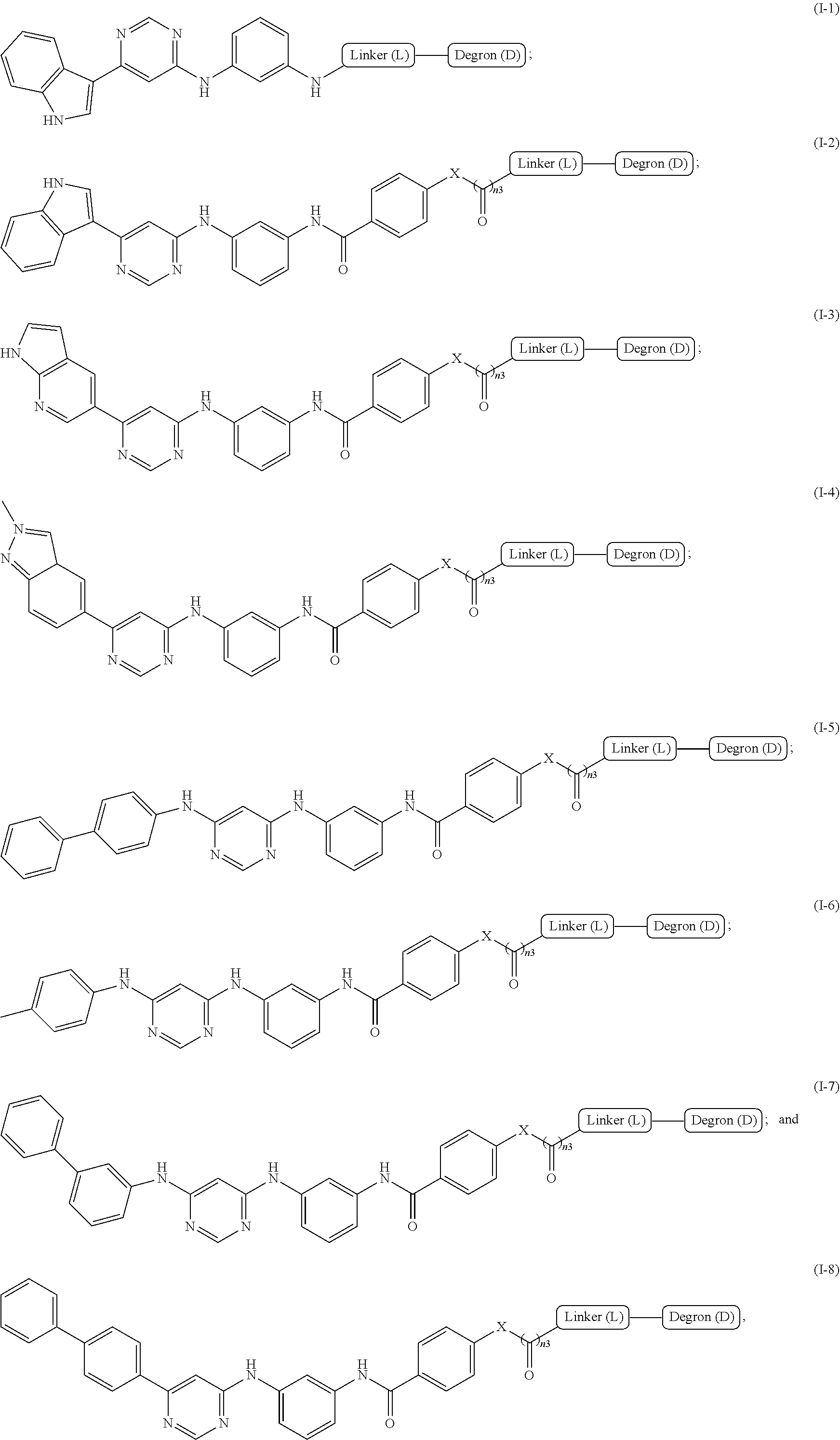

or a pharmaceutically acceptable salt or stereoisomer thereof.

### Linkers

The linker (“L”) provides a covalent attachment between the targeting ligand and the degron. The structure of linker may not be critical, provided it does not substantially interfere with the activity of the targeting ligand or the degron.

In other embodiments, the linker may include an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker includes an alkylene chain having 2-20 alkylene units. In some embodiments, the linker includes an alkylene chain having 3-12 alkylene units. In some embodiments, the linker may include a C₃-C₁₂ alkylene chain terminating in NH-group wherein the nitrogen is also bound to the degron.

In some embodiments, the linker includes an alkylene chain having 1-10 alkylene units that is interrupted by and/or terminating in

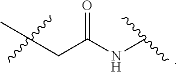 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present invention include the following:

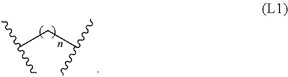

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

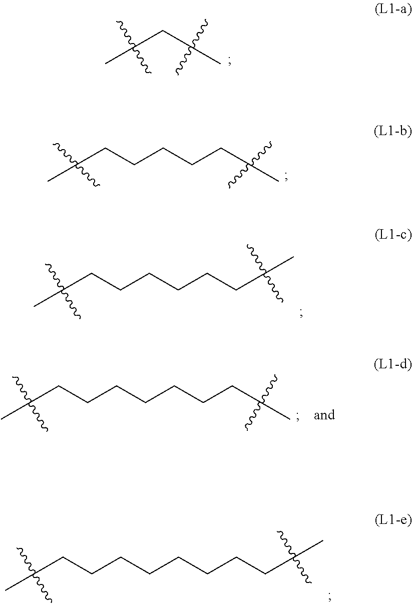

alkylene chains terminating in various functional groups (as described above), examples of which are as follows:

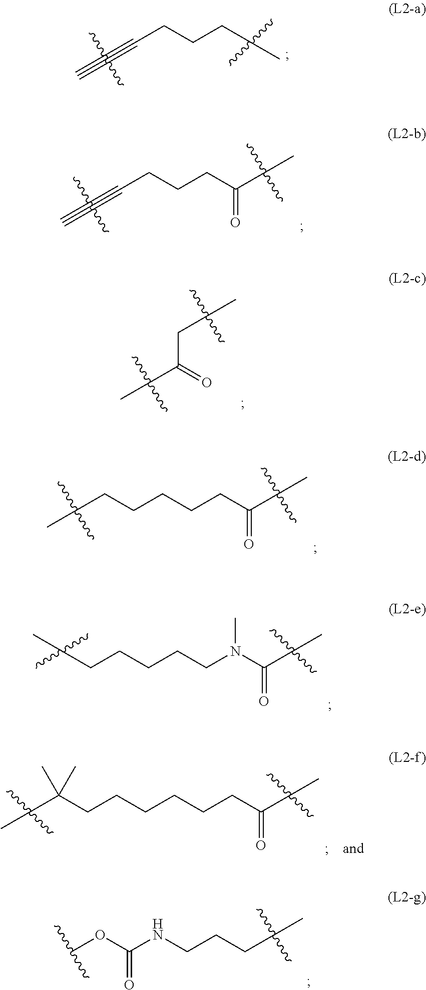

alkylene chains interrupted with various functional groups (as described above), examples of which are as follows:

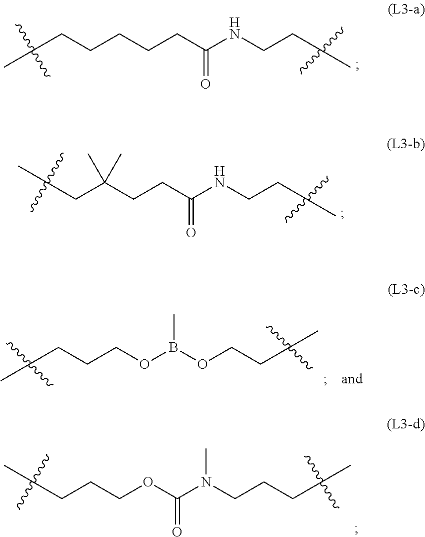 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.,

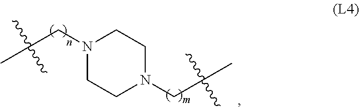

wherein m and n are independently integers of 0-10, examples of which include:

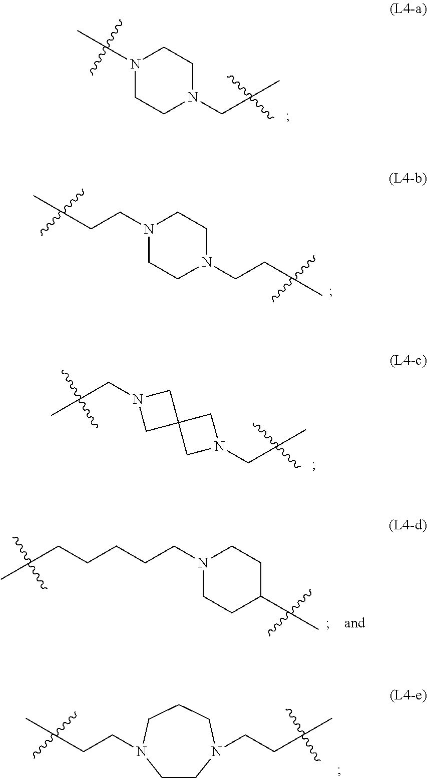 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

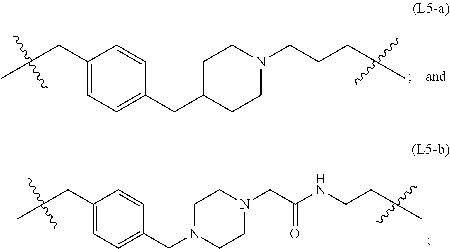 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

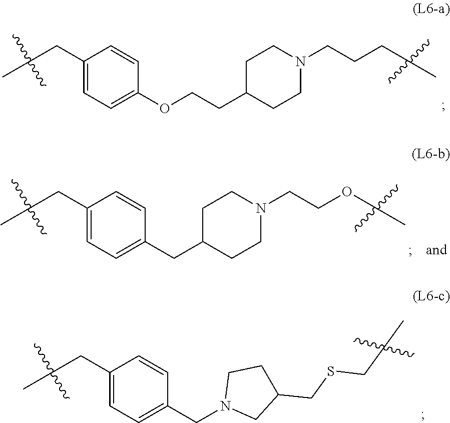 [SMILES: *CCCN1CCC(CCOc2ccc(C*)cc2)CC1 ; *Cc1ccc(CC2CCN(CCO*)CC2)cc1 ; *CSCC1CCN(Cc2ccc(C*)cc2)C1]

and
alkylene chains interrupted by a heteroatom such as N, O or B, e.g.,

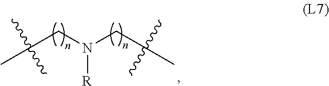

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

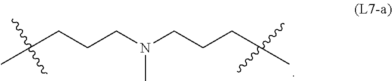 [SMILES: *CCCN(C)CCC*]

In some embodiments, the linker may include a polyethylene glycol chain that may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O), —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 1-10 PEG units. In some embodiments, the linker includes a polyethylene glycol chain having 1-6 PEG units.

In some embodiments, the linker includes a polyethylene glycol chain having 2-8 PEG units and terminating in

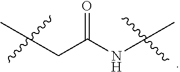 [SMILES: *CC(=O)N*]

Examples of linkers that include a polyethylene glycol chain include:

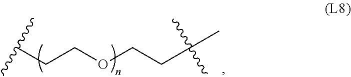

wherein n is an integer of 2-10, examples of which include:

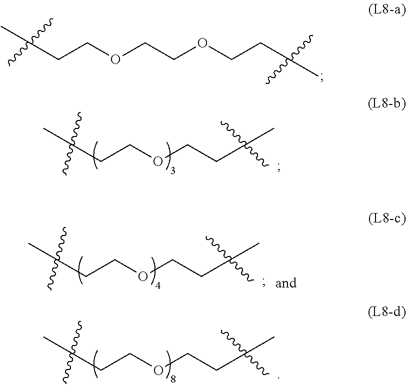

In some embodiments, the polyethylene glycol linker may terminate in a functional group, examples of which are as follows:

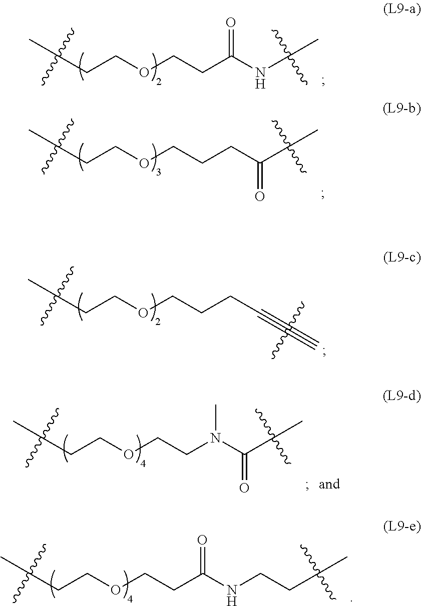

In some embodiments, the linker is represented by any one of structures L10 to L33:

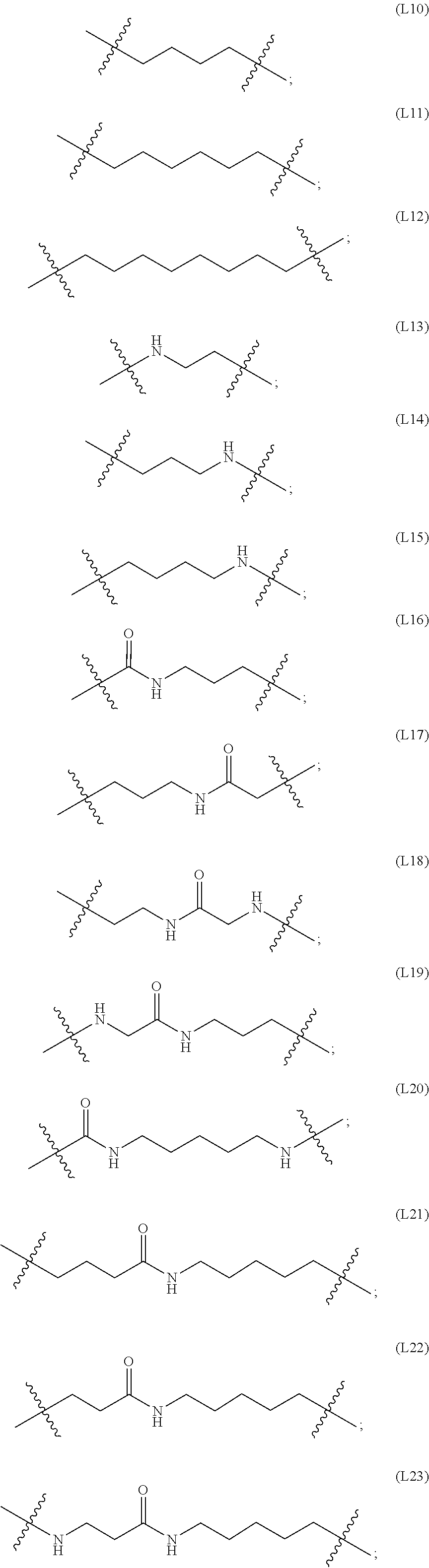

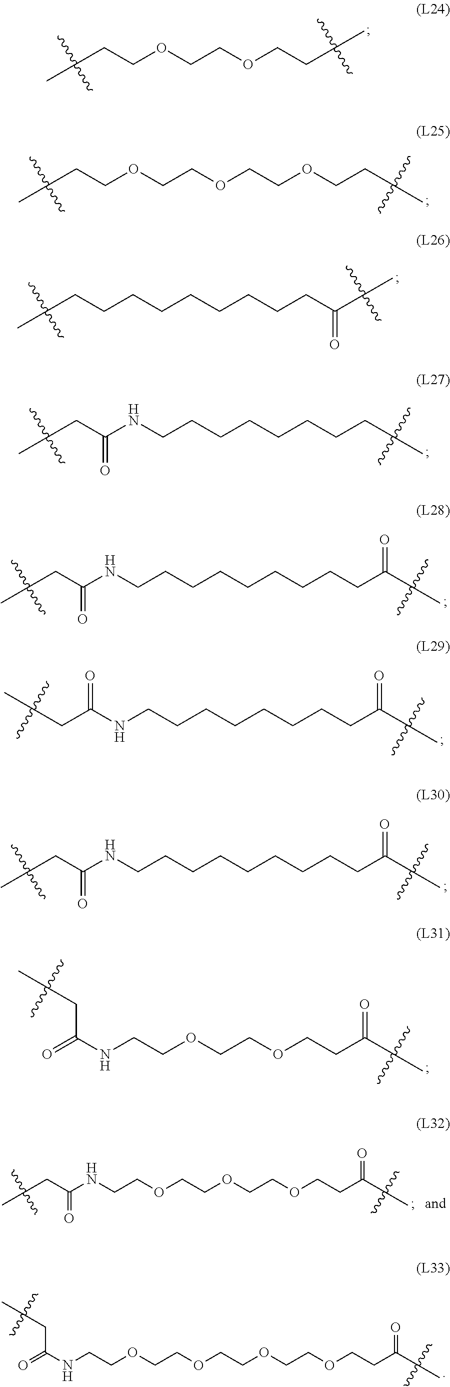

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

The degron binds the von Hippel-Lindau (VHL) E3 ubiquitin ligase.

Representative examples of such degrons may have structures represented by any one of structures (D1-a) to (D1-f):

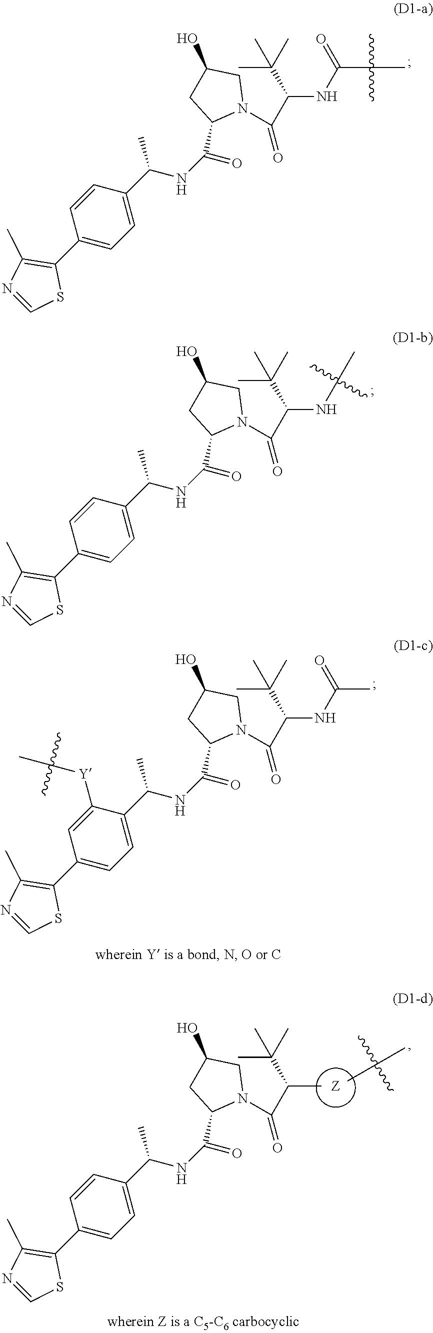

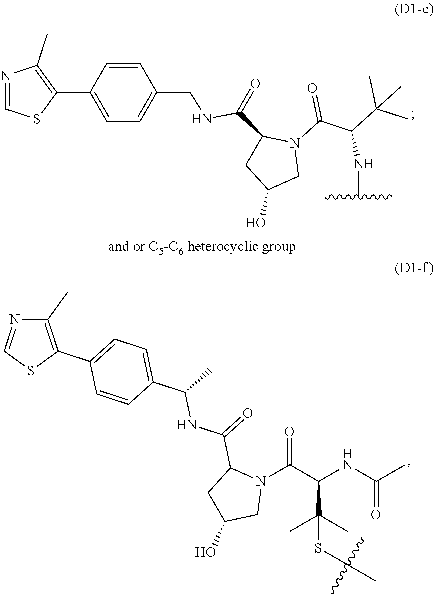 [SMILES: *N[C@H](C(=O)N1C[C@H](O)C[C@H]1C(=O)NCc1ccc(-c2scnc2C)cc1)C(C)(C)C ; *SC(C)(C)[C@H](NC(C)=O)C(=O)N1C[C@H](O)CC1C(=O)N[C@@H](C)c1ccc(-c2scnc2C)cc1]

or stereoisomer thereof. In some embodiments, Z is

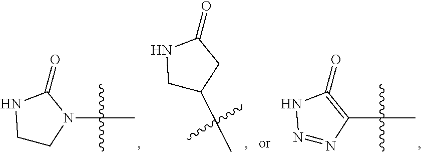

Yet other degrons that bind VHL and which may be suitable for use in the present invention are disclosed in U.S. Patent Application Publication 2017/0121321 A1.

In some embodiments, the degron binds the E3 ligase which is cereblon (CRBN).

Representative examples of such degrons have structures represented by any one of structures (D2-a) to (D2-d):

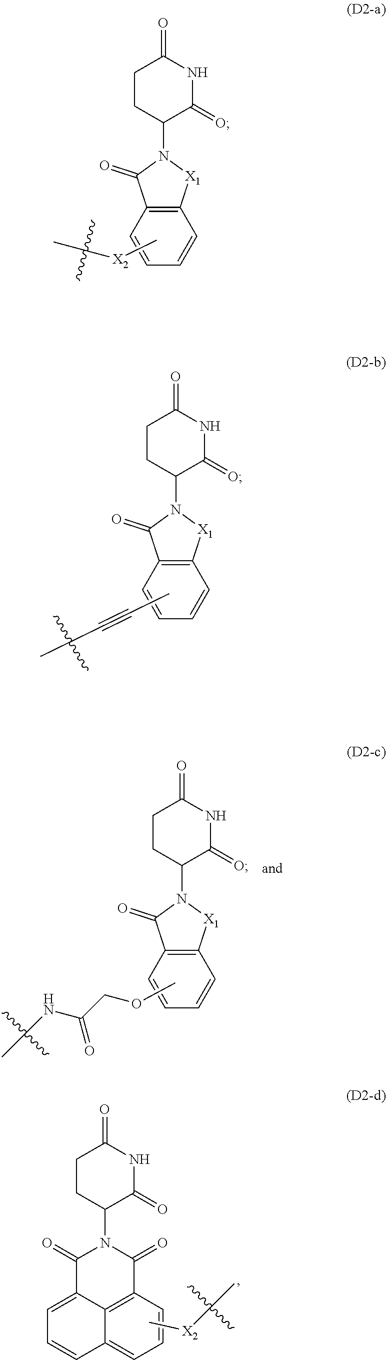

wherein X₁ is CH₂ or C(O) and X₂ is CR″₁R″₂, NR″₁R″₂, NH, O, or S, wherein R″₁ and R″₂ are independently H, halogen, OH, NH₂, C₁-C₃ alkyl, C₁-C₃ alkoxyl, or C₁-C₃ alkylamine, or R″₁ and R″₂, together with the atoms to which they are bound, form a C₃-C₇ carbocyclic or C₃-C₇ heterocyclic ring (e.g., azetidine, piperidine, pyrrolidine, cylobutane, cyclohexane).

Yet other degrons that bind cereblon and which may be suitable for use in the present invention are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

Therefore, in some embodiments, the bifunctional compounds of this invention are represented by any structures generated by the combination of any one of structures TL1 to TL8, L1 to L27, and D1-a to D1-f and D2-a to D2-d, or a pharmaceutically acceptable salt or stereoisomer thereof.

In some embodiments, the bifunctional compounds of the present invention may have any of the following structures:

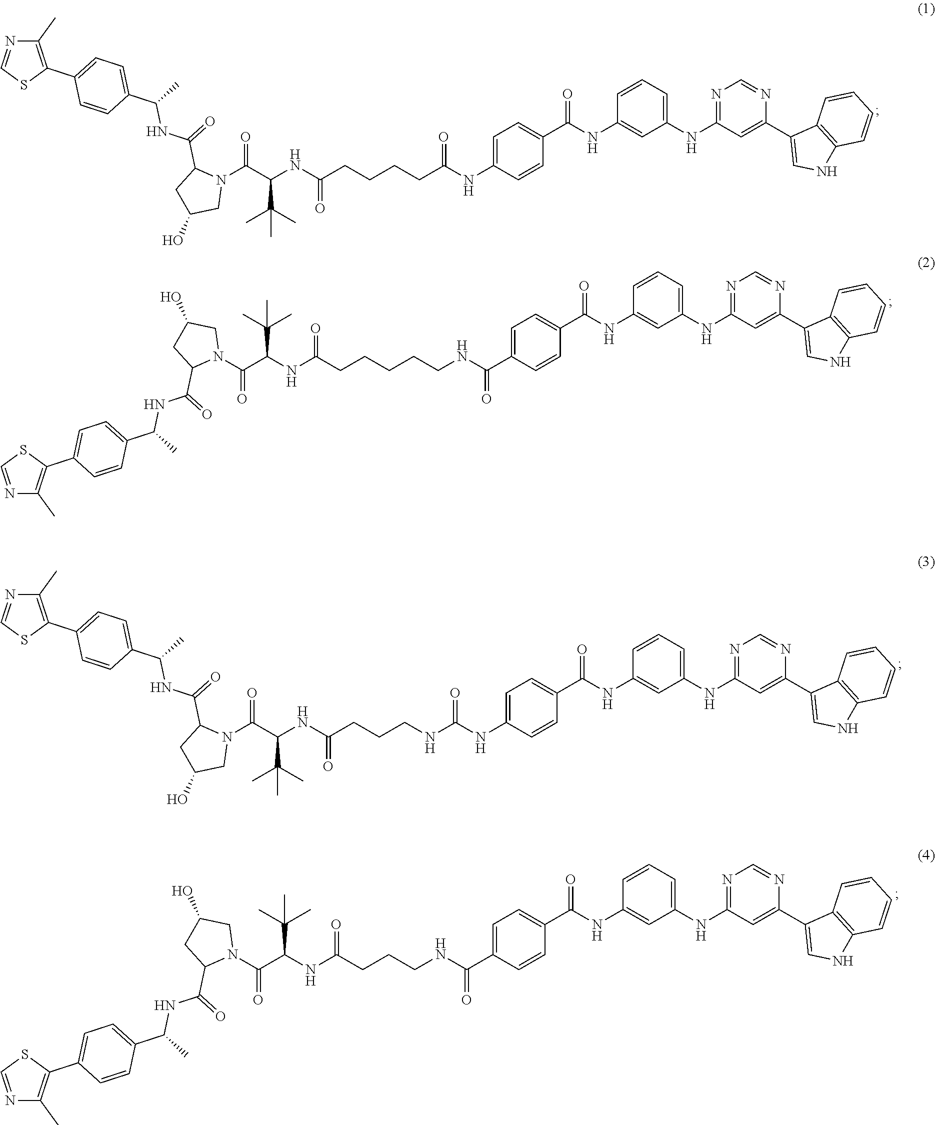

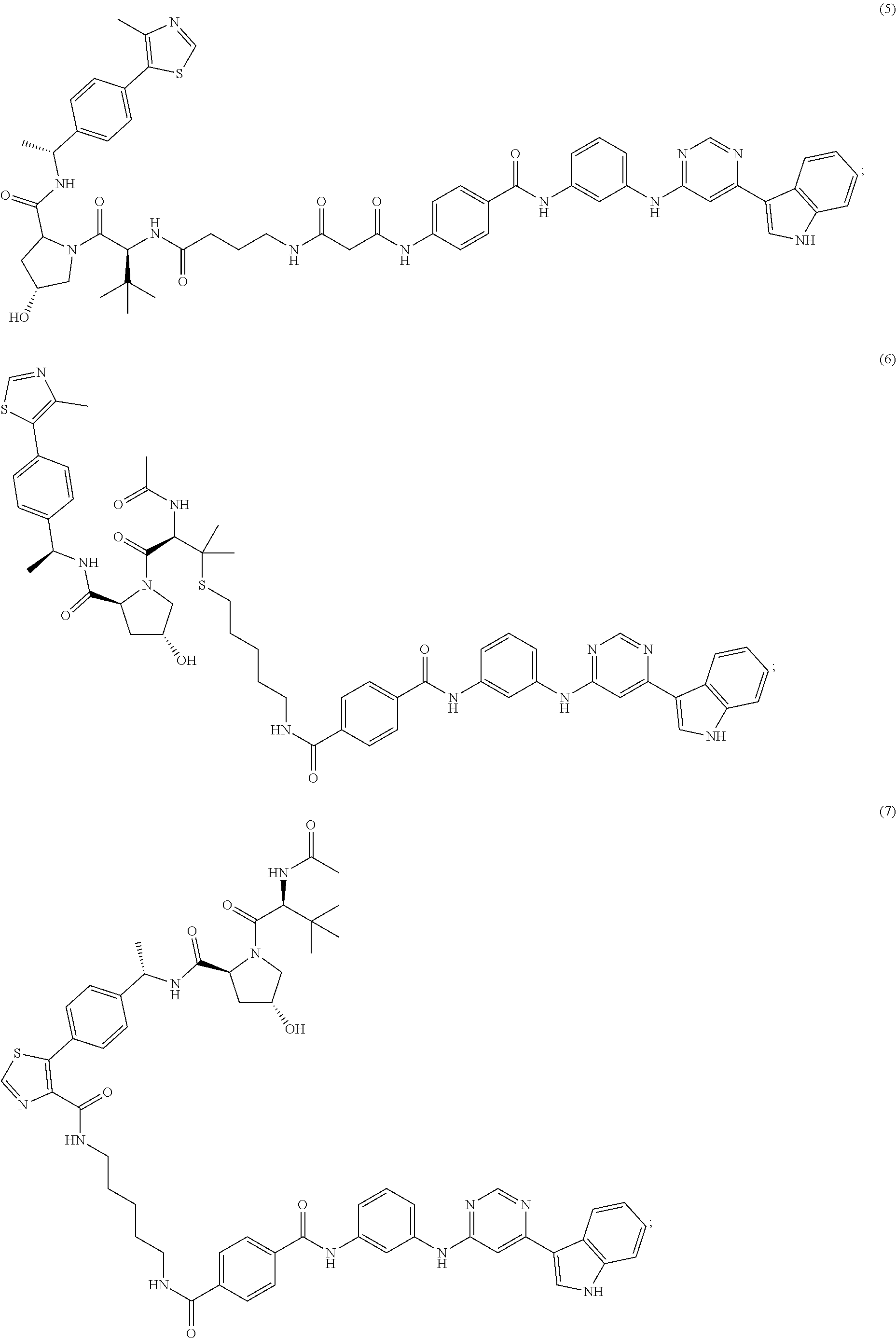 [SMILES: Cc1ncsc1-c1ccc([C@@H](C)NC(=O)C2C[C@@H](O)CN2C(=O)[C@@H](NC(=O)CCCNC(=O)CC(=O)Nc2ccc(C(=O)Nc3cccc(Nc4cc(-c5c[nH]c6ccccc56)ncn4)c3)cc2)C(C)(C)C)cc1 ; CC(=O)N[C@H](C(=O)N1C[C@H](O)C[C@H]1C(=O)N[C@@H](C)c1ccc(-c2scnc2C)cc1)C(C)(C)SCCCCCNC(=O)c1ccc(C(=O)Nc2cccc(Nc3cc(-c4c[nH]c5ccccc45)ncn3)c2)cc1 ; CC(=O)N[C@H](C(=O)N1C[C@H](O)C[C@H]1C(=O)N[C@@H](C)c1ccc(-c2scnc2C(=O)NCCCCCNC(=O)c2ccc(C(=O)Nc3cccc(Nc4cc(-c5c[nH]c6ccccc56)ncn4)c3)cc2)cc1)C(C)(C)C]

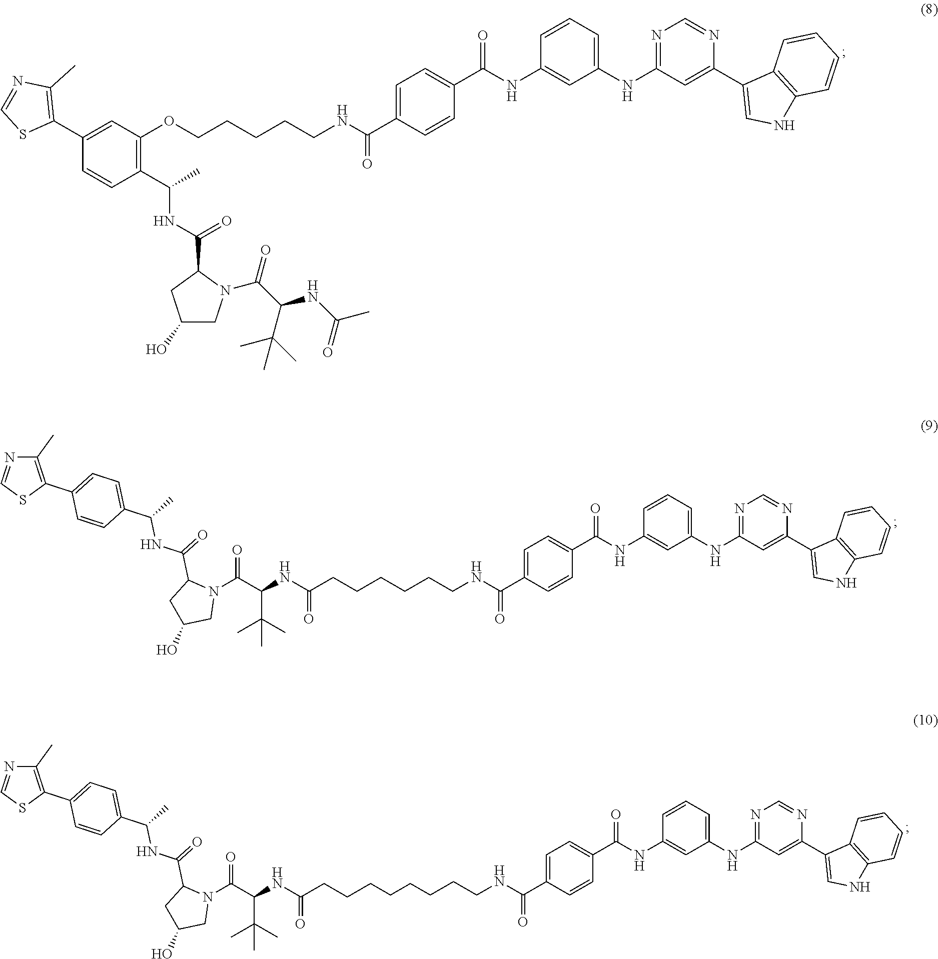 [SMILES: CC(=O)N[C@H](C(=O)N1C[C@H](O)C[C@H]1C(=O)N[C@@H](C)c1ccc(-c2scnc2C)cc1OCCCCCNC(=O)c1ccc(C(=O)Nc2cccc(Nc3cc(-c4c[nH]c5ccccc45)ncn3)c2)cc1)C(C)(C)C ; Cc1ncsc1-c1ccc([C@H](C)NC(=O)C2C[C@@H](O)CN2C(=O)[C@@H](NC(=O)CCCCCCNC(=O)c2ccc(C(=O)Nc3cccc(Nc4cc(-c5c[nH]c6ccccc56)ncn4)c3)cc2)C(C)(C)C)cc1 ; Cc1ncsc1-c1ccc([C@H](C)NC(=O)C2C[C@@H](O)CN2C(=O)[C@@H](NC(=O)CCCCCCCCNC(=O)c2ccc(C(=O)Nc3cccc(Nc4cc(-c5c[nH]c6ccccc56)ncn4)c3)cc2)C(C)(C)C)cc1]

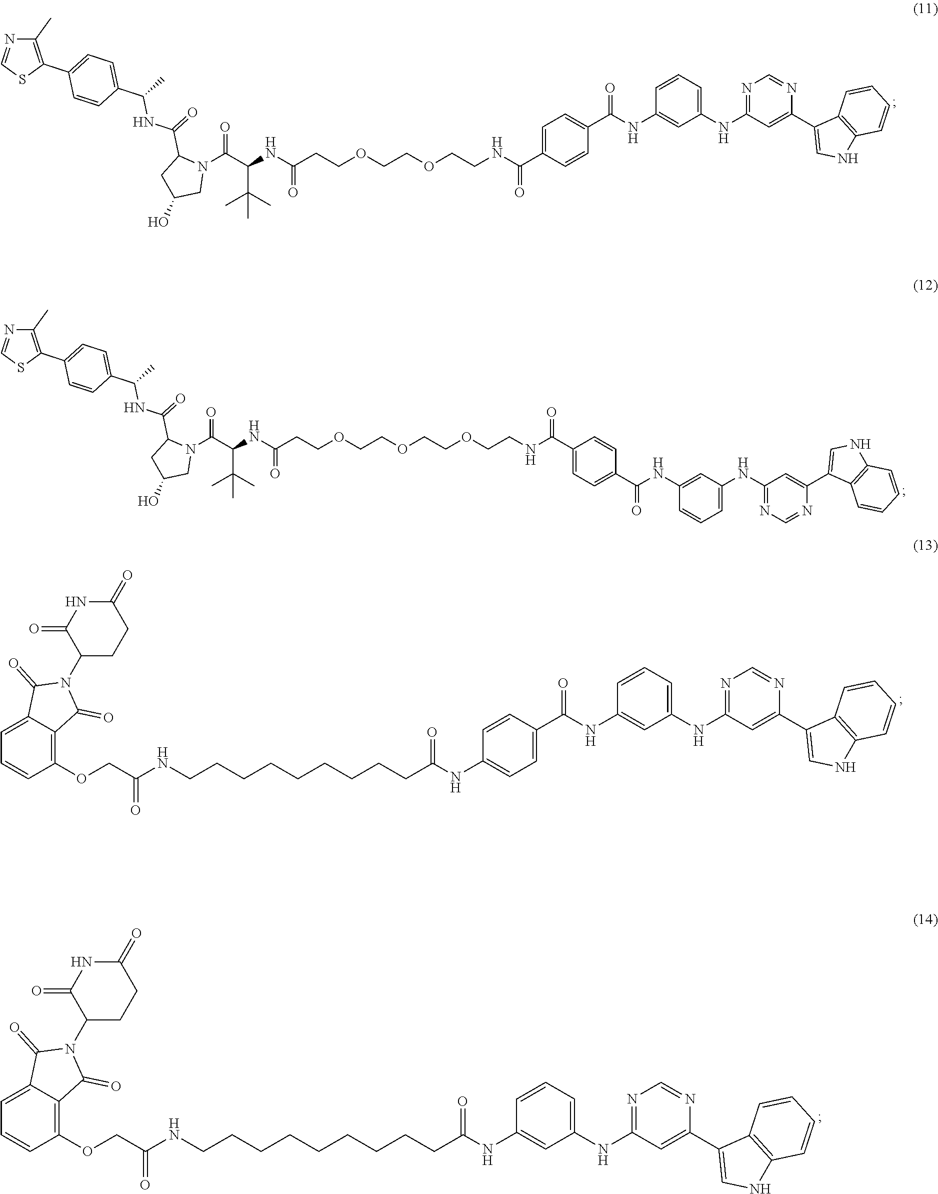

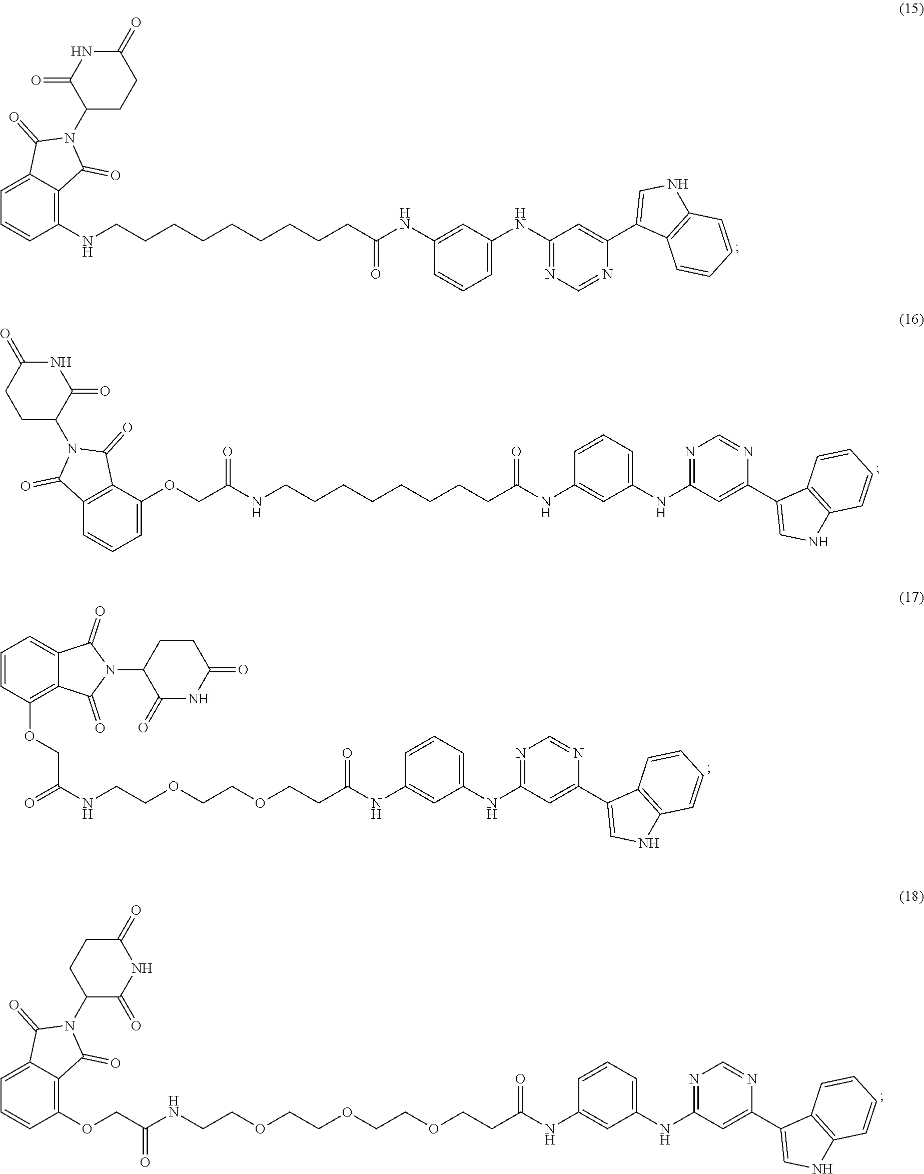 [SMILES: O=C1CCC(N2C(=O)c3cccc(NCCCCCCCCCC(=O)Nc4cccc(Nc5cc(-c6c[nH]c7ccccc67)ncn5)c4)c3C2=O)C(=O)N1 ; O=C(COc1cccc2c1C(=O)N(C1CCC(=O)NC1=O)C2=O)NCCCCCCCCC(=O)Nc1cccc(Nc2cc(-c3c[nH]c4ccccc34)ncn2)c1 ; O=C(COc1cccc2c1C(=O)N(C1CCC(=O)NC1=O)C2=O)NCCOCCOCCC(=O)Nc1cccc(Nc2cc(-c3c[nH]c4ccccc34)ncn2)c1 ; O=C(COc1cccc2c1C(=O)N(C1CCC(=O)NC1=O)C2=O)NCCOCCOCCOCCC(=O)Nc1cccc(Nc2cc(-c3c[nH]c4ccccc34)ncn2)c1]

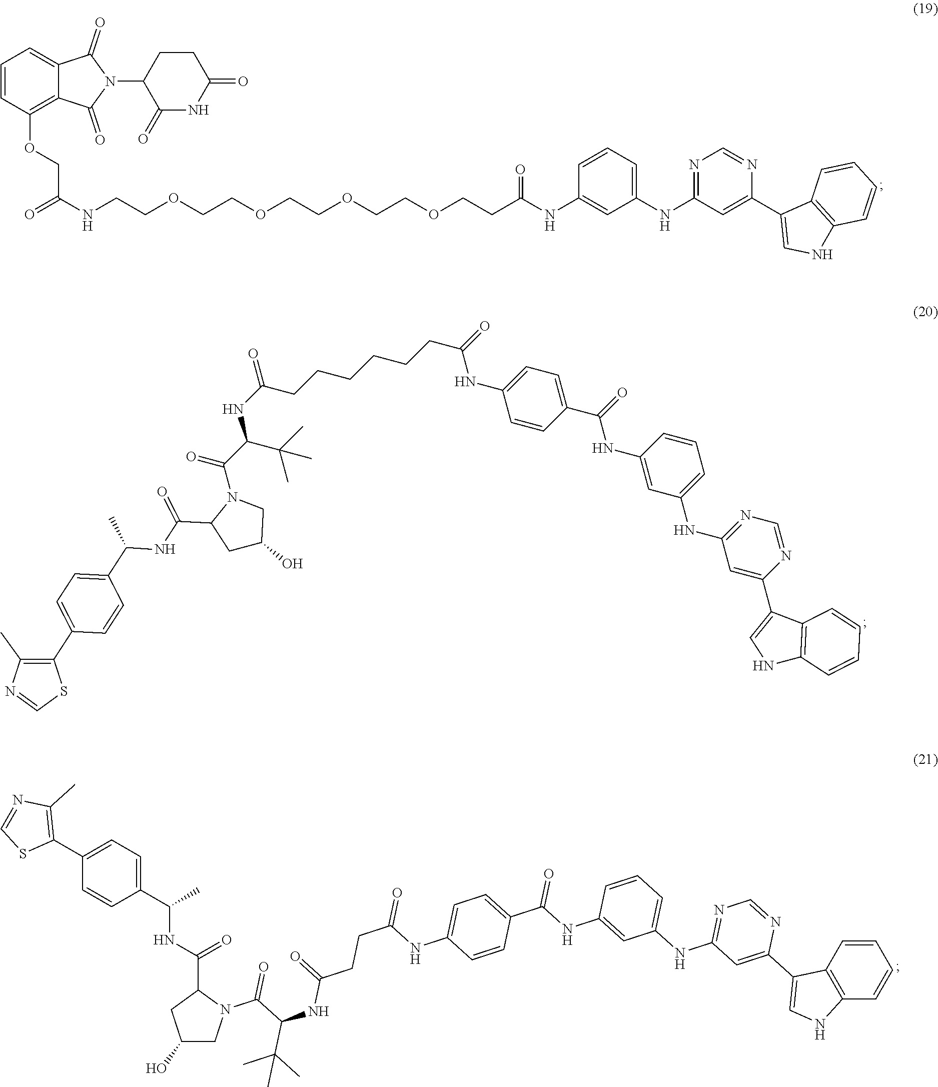 [SMILES: O=C(COc1cccc2c1C(=O)N(C1CCC(=O)NC1=O)C2=O)NCCOCCOCCOCCOCCC(=O)Nc1cccc(Nc2cc(-c3c[nH]c4ccccc34)ncn2)c1 ; Cc1ncsc1-c1ccc([C@H](C)NC(=O)C2C[C@@H](O)CN2C(=O)[C@@H](NC(=O)CCCCCCC(=O)Nc2ccc(C(=O)Nc3cccc(Nc4cc(-c5c[nH]c6ccccc56)ncn4)c3)cc2)C(C)(C)C)cc1 ; Cc1ncsc1-c1ccc([C@H](C)NC(=O)C2C[C@@H](O)CN2C(=O)[C@@H](NC(=O)CCC(=O)Nc2ccc(C(=O)Nc3cccc(Nc4cc(-c5c[nH]c6ccccc56)ncn4)c3)cc2)C(C)(C)C)cc1]

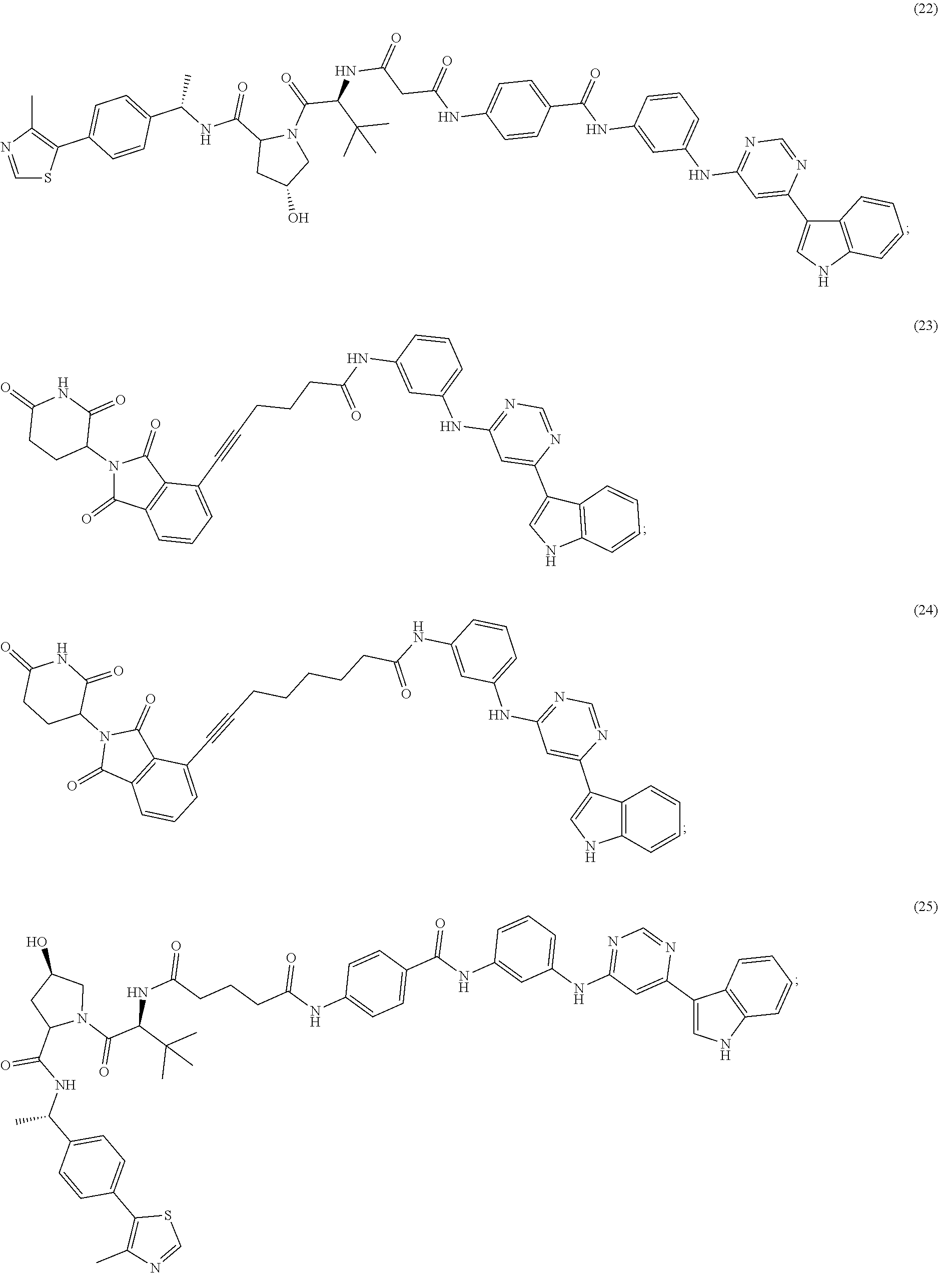 [SMILES: Cc1ncsc1-c1ccc([C@H](C)NC(=O)C2C[C@@H](O)CN2C(=O)[C@@H](NC(=O)CC(=O)Nc2ccc(C(=O)Nc3cccc(Nc4cc(-c5c[nH]c6ccccc56)ncn4)c3)cc2)C(C)(C)C)cc1 ; O=C1CCC(N2C(=O)c3cccc(C#CCCCC(=O)Nc4cccc(Nc5cc(-c6c[nH]c7ccccc67)ncn5)c4)c3C2=O)C(=O)N1 ; O=C1CCC(N2C(=O)c3cccc(C#CCCCCCC(=O)Nc4cccc(Nc5cc(-c6c[nH]c7ccccc67)ncn5)c4)c3C2=O)C(=O)N1 ; Cc1ncsc1-c1ccc([C@H](C)NC(=O)C2C[C@@H](O)CN2C(=O)[C@@H](NC(=O)CCCC(=O)Nc2ccc(C(=O)Nc3cccc(Nc4cc(-c5c[nH]c6ccccc56)ncn4)c3)cc2)C(C)(C)C)cc1]

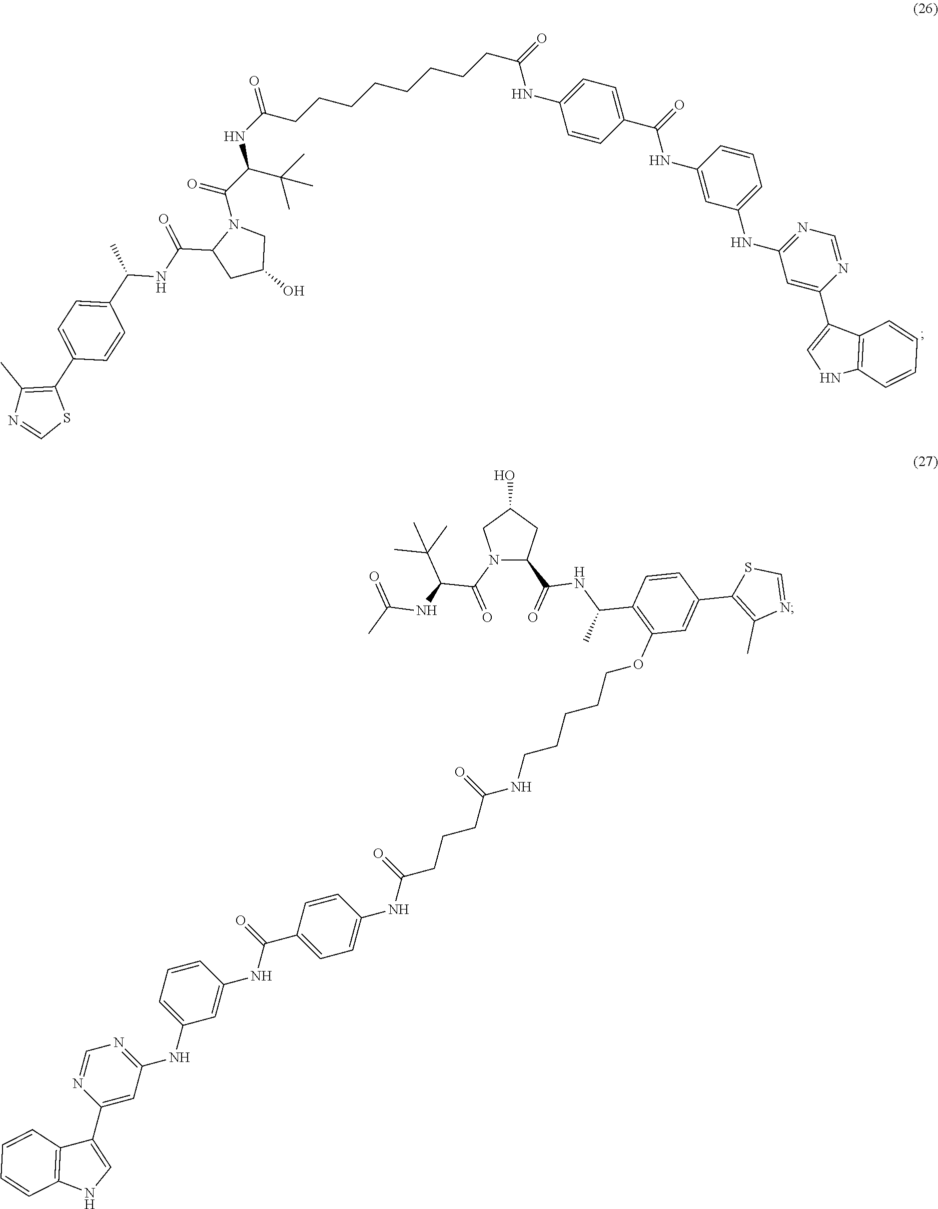 [SMILES: Cc1ncsc1-c1ccc([C@H](C)NC(=O)C2C[C@@H](O)CN2C(=O)[C@@H](NC(=O)CCCCCCCCC(=O)Nc2ccc(C(=O)Nc3cccc(Nc4cc(-c5c[nH]c6ccccc56)ncn4)c3)cc2)C(C)(C)C)cc1 ; CC(=O)N[C@H](C(=O)N1C[C@H](O)C[C@H]1C(=O)N[C@@H](C)c1ccc(-c2scnc2C)cc1OCCCCCNC(=O)CCCC(=O)Nc1ccc(C(=O)Nc2cccc(Nc3cc(-c4c[nH]c5ccccc45)ncn3)c2)cc1)C(C)(C)C]

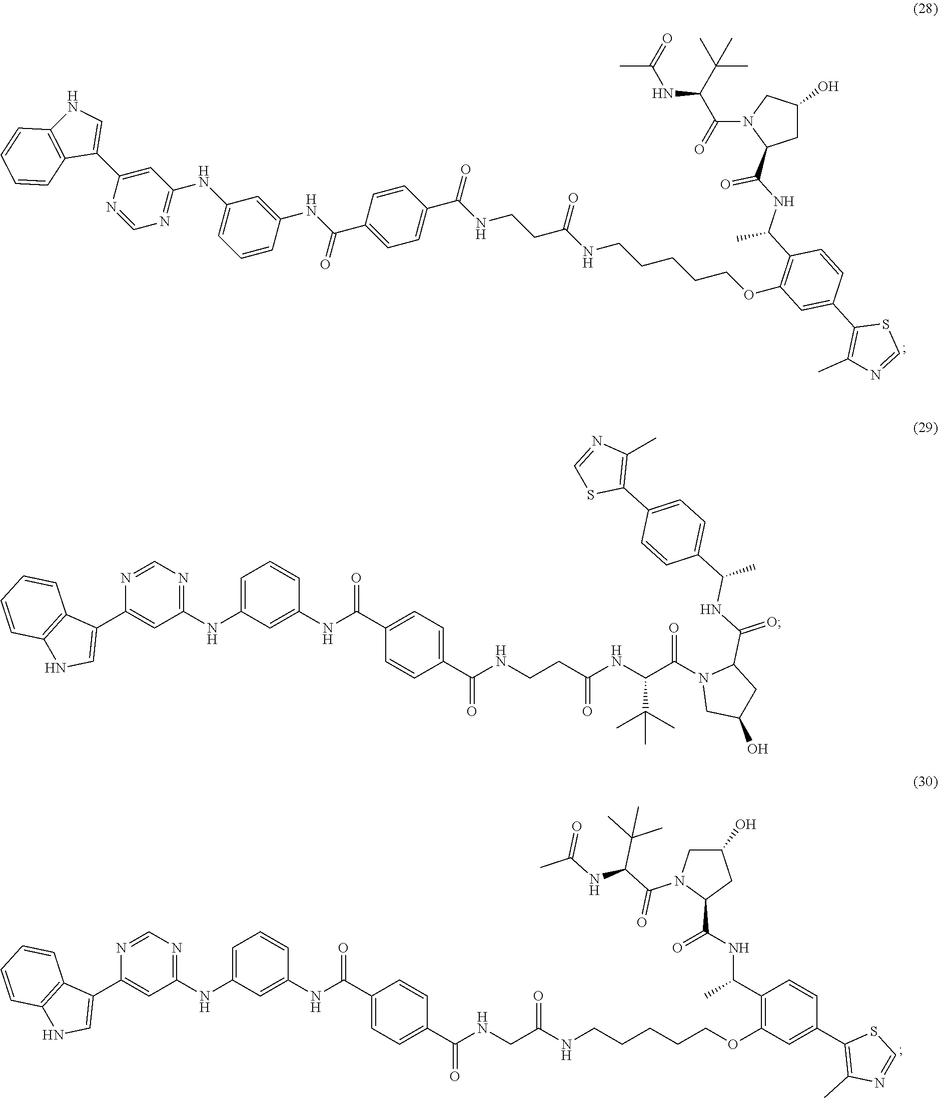

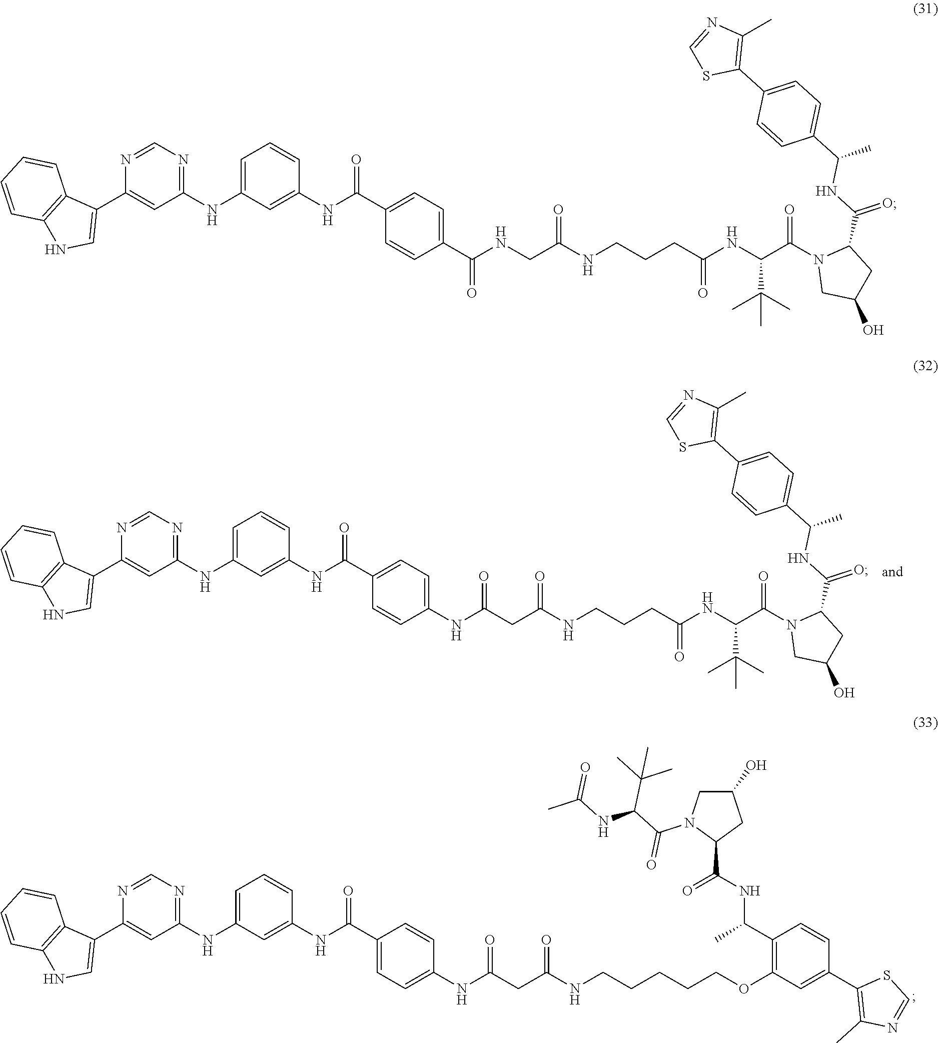

or a pharmaceutically acceptable salt or stereoisomer thereof.

Bifunctional compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bifunctional compounds of formula (I) may have at least one chiral center. Therefore, they may be in the form of a stereoisomer. As used herein, the term “stereoisomer” embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the bifunctional compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, bifunctional compounds of formula (I) embrace N-oxides, crystalline forms (also known as polymorphs), active metabolites of the compounds having the same type of activity, tautomers, and unsolvated as well as solvated and hydrated forms with pharmaceutically acceptable solvents such as water, ethanol, and the like, of the compounds. The solvated forms of the conjugates presented herein are also considered to be disclosed herein.

### Methods of Synthesis

In some embodiments, the present invention is directed to a method for making a bifunctional compound of formula (I) or a pharmaceutically acceptable salt or stereoisomer thereof. Broadly, the inventive compounds or pharmaceutically-acceptable salts or stereoisomers thereof, may be prepared by any process known to be applicable to the preparation of chemically related compounds. The compounds of the present invention will be better understood in connection with the synthetic schemes that described in various working examples that illustrate non-limiting methods by which the compounds of the invention may be prepared.

### Pharmaceutical Compositions

Another aspect of the present invention is directed to a pharmaceutical composition that includes a therapeutically effective amount of a bifunctional compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may further include one or more pharmaceutically acceptable excipients.

Broadly, bifunctional compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the present invention may be formulated in a hard or soft gelatin capsule. Representative excipients that may be used include 44egelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bifunctional compounds may be formulated for buccal or sublingual administration, examples of which include tablets, lozenges and gels.

The bifunctional compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula (I) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of formula (I) or a pharmaceutically acceptable salt or a stereoisomer thereof; or a composition including a bifunctional compound of formula (I) or a pharmaceutically acceptable salt or a stereoisomer thereof, effective in producing the desired therapeutic response in a particular patient in need thereof. Therefore, the term “therapeutically effective amount” includes the amount of a bifunctional compound of formula (I) or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer, autophagy-dependent disease (e.g., neurodegenerative disorder)) cells, or reduces the amount of at least one other of PIP4K2A, PIP4K2B, and PIP4K2C in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001).

Bifunctional compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a bifunctional compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders by modulating (e.g., reducing) the level or activity of at least one of PIP4K2A, PIP4K2B, and PIP4K2C, that entails administration of a therapeutically effective amount of a bifunctional compound formula (I) or a pharmaceutically acceptable salt or stereoisomer thereof, to a subject in need thereof.

In some embodiments, the level or activity of at least one of PIP4K2A, PIP4K2B, and PIP4K2C in the subject is reduced by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to an untreated control subject.

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Degradation or depletion of PIP4K2A, PIP4K2B, and/or PIP4K2C proteins is useful for preventing, treating and/or diagnosing cancer. In some cases, the PIP4K2C protein is targeted because it is expressed at higher levels in immune cells than the PIP4K2A and PIP4K2B proteins. Degradation or depletion of the PIP4K2A protein, the PIP4K2B protein, and/or particularly the PIP4K2C protein can enhance immune responses against cancer and tumors. Depletion or degradation of PIP4Ks, particularly PIP4K2B protein, is useful for treatment of insulin resistance.

In some embodiments, the bifunctional compounds may be used to modulate (e.g., reduce) the level of PIP4K2B in a patient suffering from or suspected of suffering from insulin resistance. In some embodiments, the bifunctional compounds may be used to modulate the level of PIP4K2C in a patient suffering from or suspected of suffering from cancer, immune deficiency, autoimmune disease, or infectious disease, or a combination thereof.

In some aspect, the present invention is directed to methods modulating (e.g., enhancing) immune functions in a subject in need thereof, comprising administering to a subject a therapeutically effective amount of the bifunctional compound of formula (I) or pharmaceutically acceptable salt or stereoisomer thereof.

In some embodiments, immune functions in the subject are enhanced by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to an untreated control subject.

In some aspect, the present invention is directed to methods of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of the bifunctional compound of formula (I) or pharmaceutically acceptable salt or stereoisomer thereof.

In some embodiments, bifunctional compounds of formula (I) and their pharmaceutically acceptable salts and stereoisomers may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the bifunctional compounds and their pharmaceutically acceptable salts and stereoisomers may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bifunctional compounds and their pharmaceutically acceptable salts and stereoisomers may be useful in the treatment of autoimmune diseases and disorders. As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome, Hashimoto thyroiditis, rheumatoid arthritis, juvenile (type 1) diabetes, polymyositis, scleroderma, Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenia purpura, psoriasis, psoriatic arthritis, dermatitis herpetiformis, ulcerative colitis, and temporal arteritis.

In other embodiments, the methods are directed to treating subjects having cancer. Broadly, the bifunctional compounds of formula (I) and their pharmaceutically acceptable salts and stereoisomers may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the bifunctional compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a bifunctional compound of the present invention may be used to treat non-metastatic or metastatic lung cancer (e.g., NSCLC, ALK-positive NSCLC, NSCLC harboring ROSI rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is leukemia, lymphoma or multiple myeloma.

The bifunctional compounds of formula (I) and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the bifunctional compounds may be administered to a patient who has received another therapy, such as chemotherapy, radioimmunotherapy, surgical therapy, immunotherapy, radiation therapy, targeted therapy or any combination thereof.

The methods of the present invention may entail administration of a bifunctional compound of formula (I) or a pharmaceutically acceptable salt or stereoisomer thereof or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

### Combination Therapy

Bifunctional compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound of formula (I) and their pharmaceutically acceptable salts and stereoisomers in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference, 60th ed., 2006. For example, anti-cancer agents that may be suitable for use in combination with the inventive bifunctional compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a bifunctional compound of formula (I) or a pharmaceutically acceptable salt or a stereoisomer thereof and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, a bifunctional compound of formula (I) or a pharmaceutically acceptable salt or stereoisomer thereof and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present bifunctional compounds and their pharmaceutically acceptable salts and stereoisomers and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bifunctional compound of formula (I) or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of N1-(4-((3-((6-(1H-indol-3-yl)pyrimidin-4-yl)amino)phenyl)carbamoyl)phenyl)-N6-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)adipamide (1)

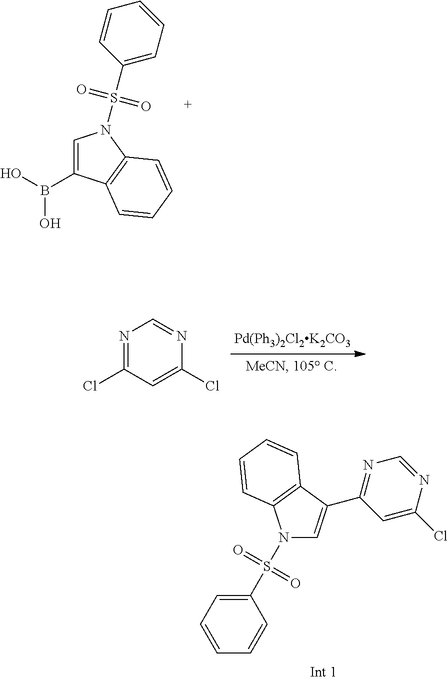 [SMILES: O=S(=O)(c1ccccc1)n1cc(B(O)O)c2ccccc21 ; Clc1cc(Cl)ncn1 ; O=S(=O)(c1ccccc1)n1cc(-c2cc(Cl)ncn2)c2ccccc21]

### 3-(6-Chloropyrimidin-4-yl)-1-(phenylsulfonyl)-1H-indole (Int 1)

4,6-Dichloropyrimidine (300 mg, 2 mmol, 1 eq.), (1-(phenylsulfonyl)-1H-indol-3-yl)boronic acid (602 mg, 2 mmol, 1 eq.), K₂CO₃ (828 mg, 3 eq.) and Pd(PPh₃)₂Cl₂ (70 mg, 0.05 eq.) were dissolved in MeCN (10 mL). The reaction mixture was heated to 105° C. for 12 hours (h) under N₂. The reaction was allowed to cool to room temperature (rt), and the solvent was removed under reduced pressure. The resulting residue was purified by flash chromatography to give Int 1 (339 mg) as a brown solid.

LC-MS (ESI) m/z: 370 [M+1]+.

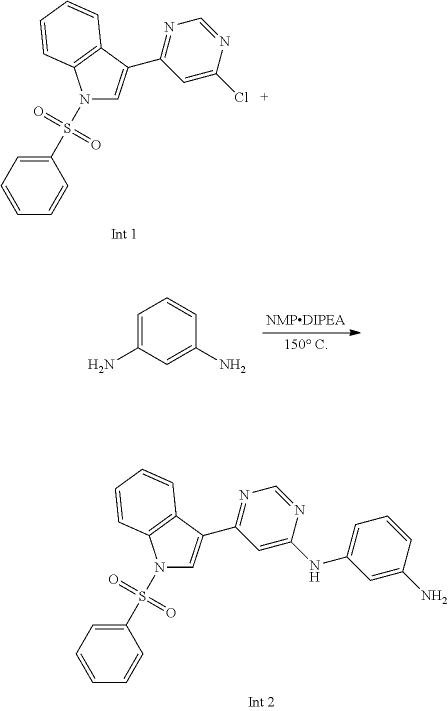 [SMILES: O=S(=O)(c1ccccc1)n1cc(-c2cc(Cl)ncn2)c2ccccc21 ; Nc1cccc(N)c1 ; Nc1cccc(Nc2cc(-c3cn(S(=O)(=O)c4ccccc4)c4ccccc34)ncn2)c1]

### N1-(6-(1-(Phenylsulfonyl)-1H-indol-3-yl)pyrimidin-4-yl)benzene-1,3-diamine (Int 2)

Int 1 (1.0 g, 2.7 mmol, 1 eq.) and phenylenediamine (1.46 g, 5 eq.) were dissolved in N-methylpyrrolidinone (NMP) (10 mL). N,N-diisopropylethylamine (DIEA) (4.7 mL, 10 eq.) was added, and the reaction mixture was stirred at 150° C. for 12 h. The resulting mixture was cooled to rt and diluted with EtOAc and water. The organic layer was washed with water and brine, dried over Na₂SO₄, and concentrated under reduced pressure. The resulting residue was purified by flash chromatography to give Int 2 (1.3 g) as a brown solid.

LC-MS (ESI) m/z: 442 [M+1]+.

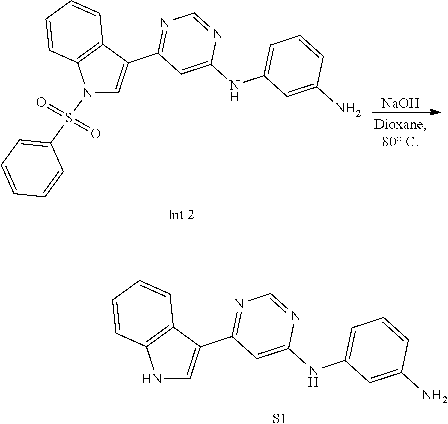

### N1-(6-(1H-Indol-3-yl)pyrimidin-4-yl)benzene-1,3-diamine (S1)

To a solution of Int 2 (300 mg, 0.67 mmol, 1 eq.) in dioxane (3 mL) was added 2M NaOH (2 mL), and the reaction mixture was stirred at 80° C. for 3 h. The mixture was cooled to rt, acidified to pH 5˜7 with 2M HCl, and extracted with EtOAc. The organic layer was washed with water and brine and concentrated under reduced pressure. The resulting residue was purified by flash chromatography to give S1 (167 mg) as a yellow solid.

LC-MS (ESI) m/z: 302 [M+1]+.

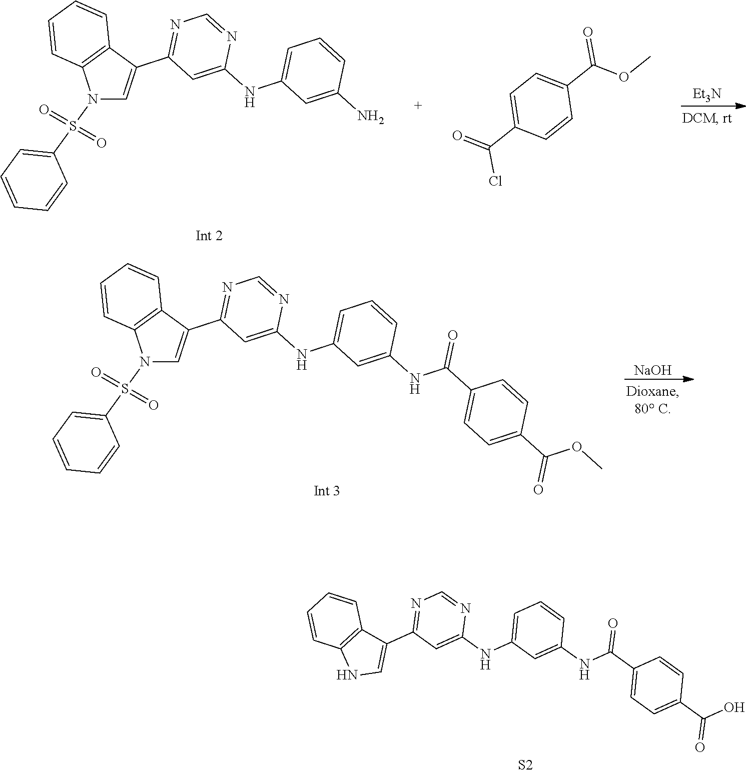

### 4-((3-((6-(1H-Indol-3-yl)pyrimidin-4-yl)amino)phenyl)carbamoyl)benzoic acid (S2)

To a solution of Int 2 (100 mg, 0.23 mmol, 1 eq.) and Et₃N (63 μL) in DCM (2 mL) was added methyl 4-(chlorocarbonyl)benzoate (50 mg, 2 eq.) at 0° C. The reaction mixture was stirred at rt overnight. The solvent was removed under reduced pressure. The resulting residue was diluted with water and extracted with EtOAc. The organic layer was washed with water and brine, dried over Na₂SO₄, and concentrated under reduced pressure. The resulting residue was purified by flash chromatography to give Int 3 (120 mg) as a yellow solid. LC-MS (ESI) m/z: 604 [M+1]+.

To a solution of Int 3 (120 mg, 1 eq.) in dioxane (2 mL) was added 2M NaOH (1 mL), and the reaction mixture was stirred at 80ºC for 3 h. The resulting mixture was cooled to rt, acidified to pH 5˜7 with 2M HCl, and extracted with EtOAc. The organic layer was washed with water and brine, dried over Na₂SO₄, and evaporated under reduced pressure. The resulting residue was purified by flash chromatography to give S2 (57 mg) as a yellow solid.

LC-MS (ESI) m/z: 450 [M+1]+.

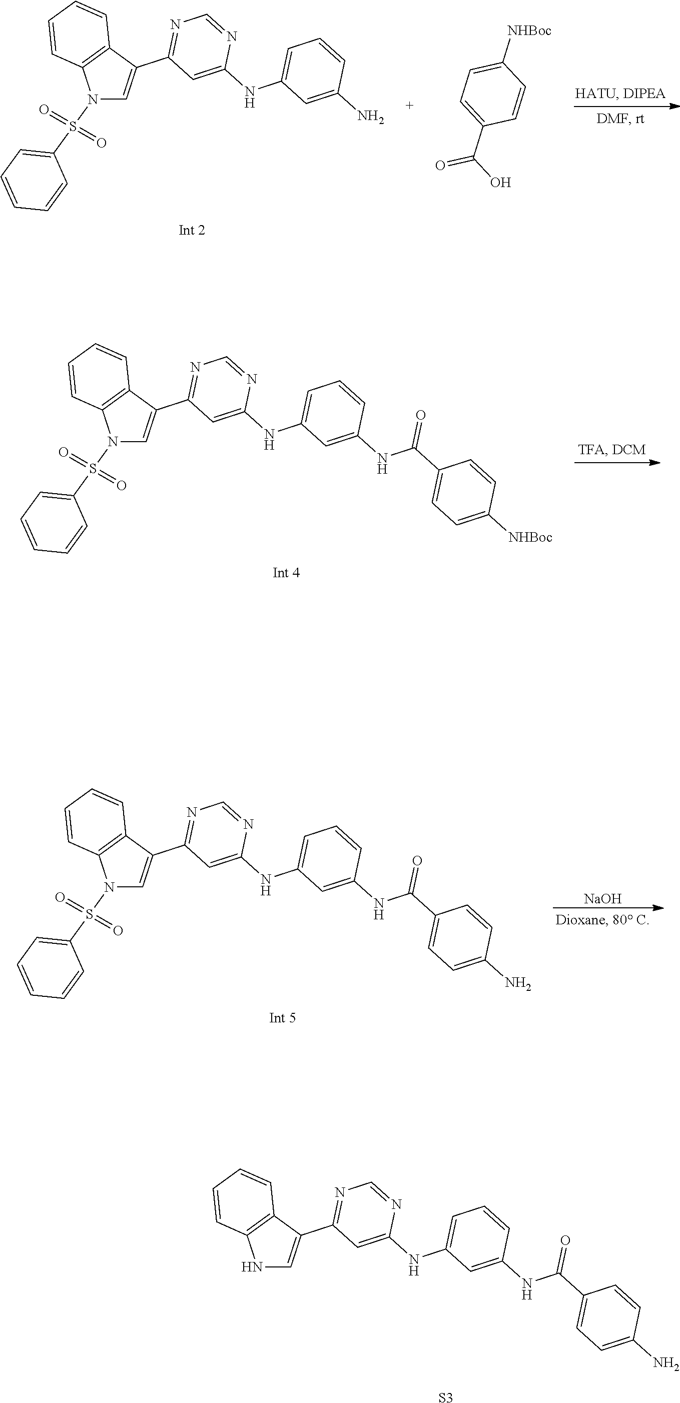

### N1-(6-(1H-Indol-3-yl)pyrimidin-4-yl)benzene-1,3-diamine (S3)

To a mixture of Int 2 (88 mg, 1.0 eq.), 4-((tert-butoxycarbonyl)amino)benzoic acid (71 mg, 1.5 eq.), and DIEA (4.0 eq.) in DMF (2 mL) was added hexafluorophosphate azabenzotriazole tetramethyl uranium (HATU) (114 mg, 1.5 eq). The resulting mixture was stirred at rt overnight, and the solvent was removed under reduced pressure. The resulting residue was purified by flash chromatography to give Int 4 (101 mg) as a yellow solid.

LC-MS (ESI) m/z: 661 [M+1]+.

To a solution of Int 4 (101 mg, 1 eq.) in DCM (2 mL) was added TFA (1 mL), and the reaction mixture was stirred at rt for 3 h. The solvent was removed under reduced pressure to give crude product Int 5 which was used in the next step without further purification.

LC-MS (ESI) m/z: 561 [M+1]+.

To a solution of Int 5 in dioxane (2 mL) was added 2M NaOH (1 mL). The mixture was stirred at 80ºC for 3 h. The reaction mixture was cooled to rt, acidified to pH 5˜7 with 2M HCl, and extracted with EtOAc. The organic layer was washed with water and brine and concentrated under reduced pressure. The resulting residue was purified by flash chromatography to give S3 (57 mg) as a yellow solid.

LC-MS (ESI) m/z: 421 [M+1]+.

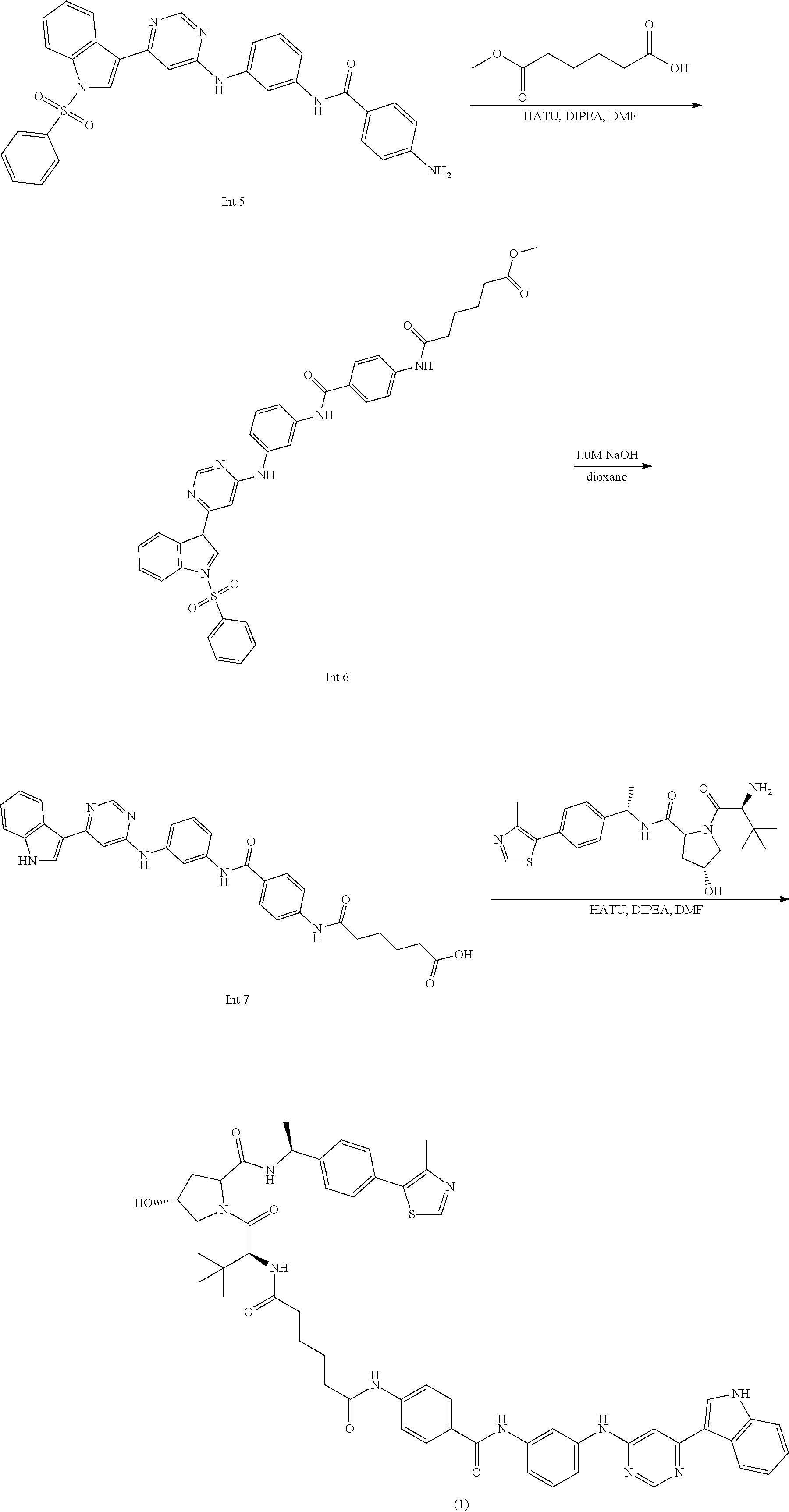

To a solution of Int 5 (56 mg, 0.1 mmol, 1 eq.) was in DMF (1 mL) was added 6-methoxy-6-oxohexanoic acid (19.2 mg, 1.2 eq.), HATU (76 mg, 2 eq.), and DIEA (64 mg, 5 eq.), and the reaction mixture was stirred at rt overnight. The crude was purified via prep-HPLC to yield the Int 6 (57 mg, 81%) as a yellow solid.

LC-MS (ESI) m/z: 703 [M+1]+.

To a solution of Int 6 (10 mg, 0.018 mmol, 1 eq.) in 1,4-dioxane (0.5 mL) was added 1.0 N NaOH (0.5 mL), and the reaction was stirred at rt for 5 h. The crude mixture was purified by prep-HPLC to yield the Int 7 (38 mg, 88%) as an ochre solid.

LC-MS (ESI) m/z: 549 [M+1]+.

To a solution of Int 7 (10 mg, 0.018 mmol, 1.2 eq.) in DMF was added (4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (7 mg, 1 eq.), HATU (11 mg, 2 eq.), and DIEA (10 mg, 5 eq.), and the reaction mixture was stirred at rt overnight. The crude mixture was purified via prep-HPLC to yield bifunctional compound 1 (6.1 mg, 42%) as a light yellow solid.

LC-MS (ESI) m/z 976.20 [M+H]+.

¹H NMR (500 MHZ, DMSO-d₆) δ 12.25 (s, 1H), 10.77 (s, 1H), 10.26 (d, J=8.3 Hz, 1H), 10.18 (s, 1H), 8.99 (s, 1H), 8.86 (s, 1H), 8.40-8.31 (m, 2H), 8.28 (d, J=2.2 Hz, 1H), 8.04 (d, J=7.8 Hz, 1H), 8.00-7.91 (m, 2H), 7.83 (d, J=9.2 Hz, 1H), 7.76 (d, J=8.6 Hz, 2H), 7.61 (d, J=7.8 Hz, 1H), 7.53 (dd, J=8.3, 3.8 Hz, 1H), 7.48 (d, J=8.3 Hz, 1H), 7.46-7.36 (m, 6H), 7.32 (q, J=8.5, 7.6 Hz, 2H), 4.98-4.85 (m, 1H), 4.53 (d, J=9.3 Hz, 1H), 4.43 (t, J=8.0 Hz, 1H), 4.29 (q, J=3.6 Hz, 1H), 3.64-3.62 (m, 1H), 2.46 (s, 3H), 2.36 (q, J=8.2, 7.4 Hz, 2H), 2.33-2.26 (m, 1H), 2.18 (dt, J=13.9, 6.9 Hz, 1H), 2.08 (s, 1H), 2.02 (ddd, J=11.0, 7.8, 2.8 Hz, 1H), 1.80 (ddd, J=12.9, 8.5, 4.6 Hz, 1H), 1.57 (ddd, J=26.6, 14.1, 7.5 Hz, 4H), 1.38 (d, J=7.0 Hz, 3H), 0.94 (d, J=7.7 Hz, 9H).

### Example 2: Synthesis of N1-(3-((6-(1H-indol-3-yl)pyrimidin-4-yl)amino)phenyl)-N4-(4-(((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)terephthalamide (4)

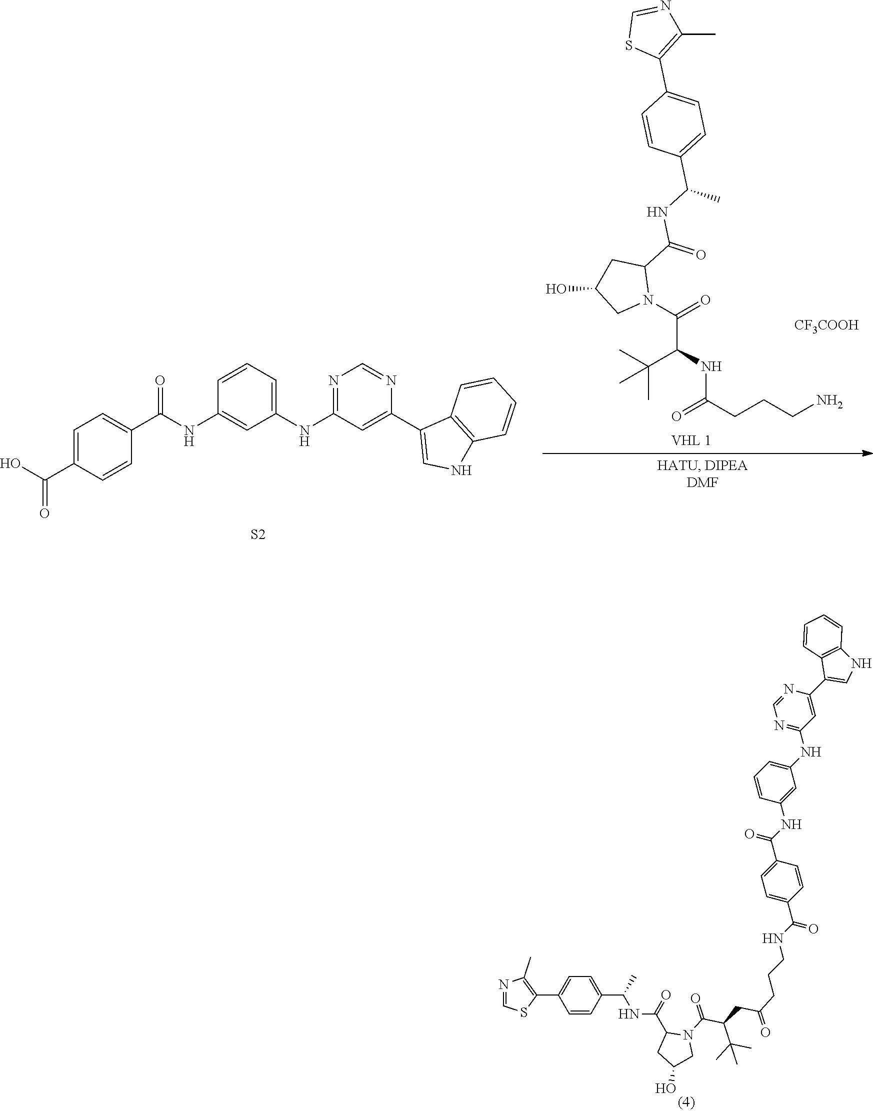 [SMILES: O=C(O)c1ccc(C(=O)Nc2cccc(Nc3cc(-c4c[nH]c5ccccc45)ncn3)c2)cc1 ; Cc1ncsc1-c1ccc([C@H](C)NC(=O)C2C[C@@H](O)CN2C(=O)[C@@H](NC(=O)CCCN)C(C)(C)C)cc1 ; "CF3COOH" ; "S2" ; Cc1ncsc1-c1ccc([C@H](C)NC(=O)C2C[C@@H](O)CN2C(=O)[C@@H](CC(=O)CCCNC(=O)c2ccc(C(=O)Nc3cccc(Nc4cc(-c5c[nH]c6ccccc56)ncn4)c3)cc2)C(C)(C)C)cc1]

To a mixture of S2 (9.0 mg, 1.0 eq.), VHL1 (13 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 4 (6.1 mg, 32%) as a white solid.

LC-MS (ESI) m/z: 961 [M+1]+.

¹H NMR (500 MHZ, DMSO-d₆) δ 12.32 (s, 1H), 10.90 (s, 1H), 10.53 (s, 1H), 8.99 (s, 1H), 8.88 (s, 1H), 8.66 (t, J=5.6 Hz, 1H), 8.41-8.28 (m, 3H), 8.10-7.95 (m, 5H), 7.91 (d, J=9.3 Hz, 1H), 7.66-7.25 (m, 11H), 4.93 (t, J=7.2 Hz, 2H), 4.62-4.39 (m, 4H), 4.29 (q, J=3.5 Hz, 2H), 3.68-3.57 (m, 4H), 3.29 (t, J=6.6 Hz, 3H), 2.46 (s, 3H), 2.29 (ddd, J=49.1, 14.4, 7.1 Hz, 3H), 2.02 (t, J=10.3 Hz, 1H), 1.85-1.71 (m, 3H), 1.38 (d, J=7.0 Hz, 3H), 0.95 (d, J=7.0 Hz, 9H).

### Example 3: Synthesis of N1-(3-((6-(1H-indol-3-yl)pyrimidin-4-yl)amino)phenyl)-N4-(5-(((R)-3-acetamido-4-((2S,4R)-4-hydroxy-2-(((S)-1-(4-(4-methylthiazol-5-yl)phenyl)ethyl)carbamoyl)pyrrolidin-1-yl)-2-methyl-4-oxobutan-2-yl)thio)pentyl)terephthalamidede (6)

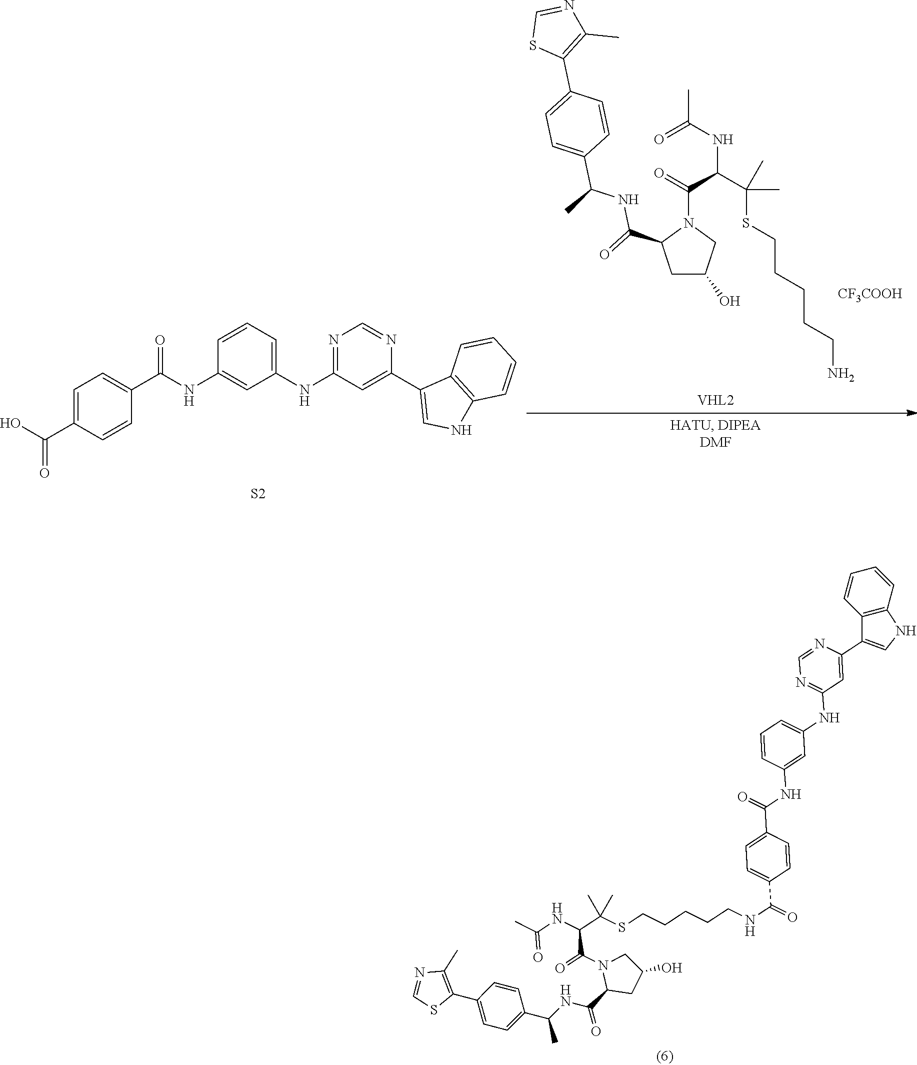

To a mixture of S2 (9.0 mg, 1.0 eq.), VHL2 (15 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 6 (7.0 mg, 34%) as a white solid.

LC-MS (ESI) m/z: 1021 [M+1]+.

¹H NMR (500 MHz, DMSO-d₆) δ 12.32 (s, 1H), 10.90 (s, 1H), 10.52 (s, 1H), 8.98 (s, 1H), 8.88 (s, 1H), 8.62 (t, J=5.6 Hz, 1H), 8.37 (d, J=3.1 Hz, 1H), 8.30 (d, J=2.1 Hz, 1H), 8.21 (d, J=7.9 Hz, 1H), 8.12 (d, J=9.5 Hz, 1H), 8.09-7.95 (m, 5H), 7.66-7.24 (m, 11H), 5.00-4.78 (m, 3H), 4.41 (t, J=8.0 Hz, 2H), 4.30 (s, 2H), 3.28 (q, J=6.6 Hz, 3H), 2.56 (q, J=7.2 Hz, 2H), 2.45 (s, 3H), 2.10-2.00 (m, 1H), 1.89 (s, 3H), 1.52 (dt, J=29.6, 7.4 Hz, 4H), 1.43-1.31 (m, 8H), 1.28 (s, 3H).

### Example 4: Synthesis of N1-(3-((6-(1H-indol-3-yl)pyrimidin-4-yl)amino)phenyl)-N4-(5-(5-(4-((S)-1-((2S,4R)-1-((S)-2-acetamido-3,3-dimethylbutanoyl)-4-hydroxypyrrolidine-2 carboxamido)ethyl)phenyl)thiazole-4-carboxamido)pentyl)terephthalamide (7)

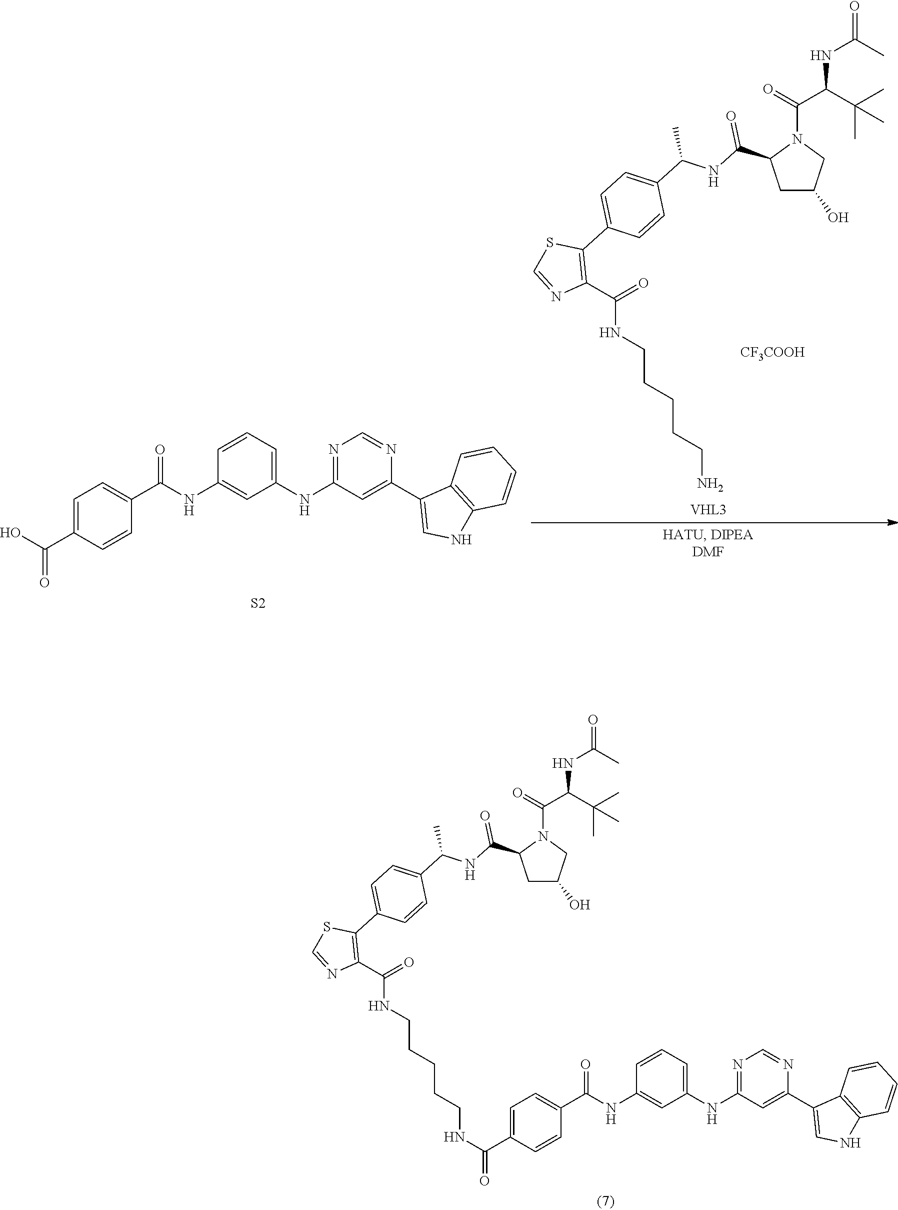 [SMILES: O=C(O)c1ccc(C(=O)Nc2cccc(Nc3cc(-c4c[nH]c5ccccc45)ncn3)c2)cc1 ; CC(=O)N[C@H](C(=O)N1C[C@H](O)C[C@H]1C(=O)N[C@@H](C)c1ccc(-c2scnc2C(=O)NCCCCCN)cc1)C(C)(C)C ; "CF3COOH" ; "S2" ; CC(=O)N[C@H](C(=O)N1C[C@H](O)C[C@H]1C(=O)N[C@@H](C)c1ccc(-c2scnc2C(=O)NCCCCCNC(=O)c2ccc(C(=O)Nc3cccc(Nc4cc(-c5c[nH]c6ccccc56)ncn4)c3)cc2)cc1)C(C)(C)C]

To a mixture of S2 (9.0 mg, 1.0 eq.), VHL3 (15 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 7 (7.2 mg, 35%) as a white solid.

LC-MS (ESI) m/z: 1032 [M+1]+.

¹H NMR (500 MHZ, DMSO-d₆) δ 12.22 (s, 1H), 10.68 (s, 1H), 10.50 (s, 1H), 9.10 (s, 1H), 8.84 (s, 1H), 8.62 (t, J=5.6 Hz, 1H), 8.45 (t, J=5.9 Hz, 1H), 8.39 (d, J=7.8 Hz, 1H), 8.33 (d, J=3.1 Hz, 1H), 8.29 (d, J=2.1 Hz, 1H), 8.05 (d, J=8.3 Hz, 3H), 7.99 (d, J=8.3 Hz, 2H), 7.89 (d, J=9.3 Hz, 1H), 7.64-7.54 (m, 2H), 7.53-7.46 (m, 3H), 7.46-7.38 (m, 2H), 7.36-7.22 (m, 4H), 4.92 (p, J=6.9 Hz, 2H), 4.56-4.40 (m, 2H), 4.28 (q, J=3.6 Hz, 1H), 3.25 (dq, J=32.4, 6.7 Hz, 5H), 2.07-1.98 (m, 1H), 1.88 (s, 3H), 1.81 (ddd, J=12.9, 8.5, 4.7 Hz, 2H), 1.62-1.45 (m, 5H), 1.36 (dd, J=15.1, 7.3 Hz, 5H), 0.93 (d, J=5.0 Hz, 9H).

### Example 5: Synthesis of N1-(3-((6-(1H-indol-3-yl)pyrimidin-4-yl)amino)phenyl)-N4-(5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)terephthalamide (8)

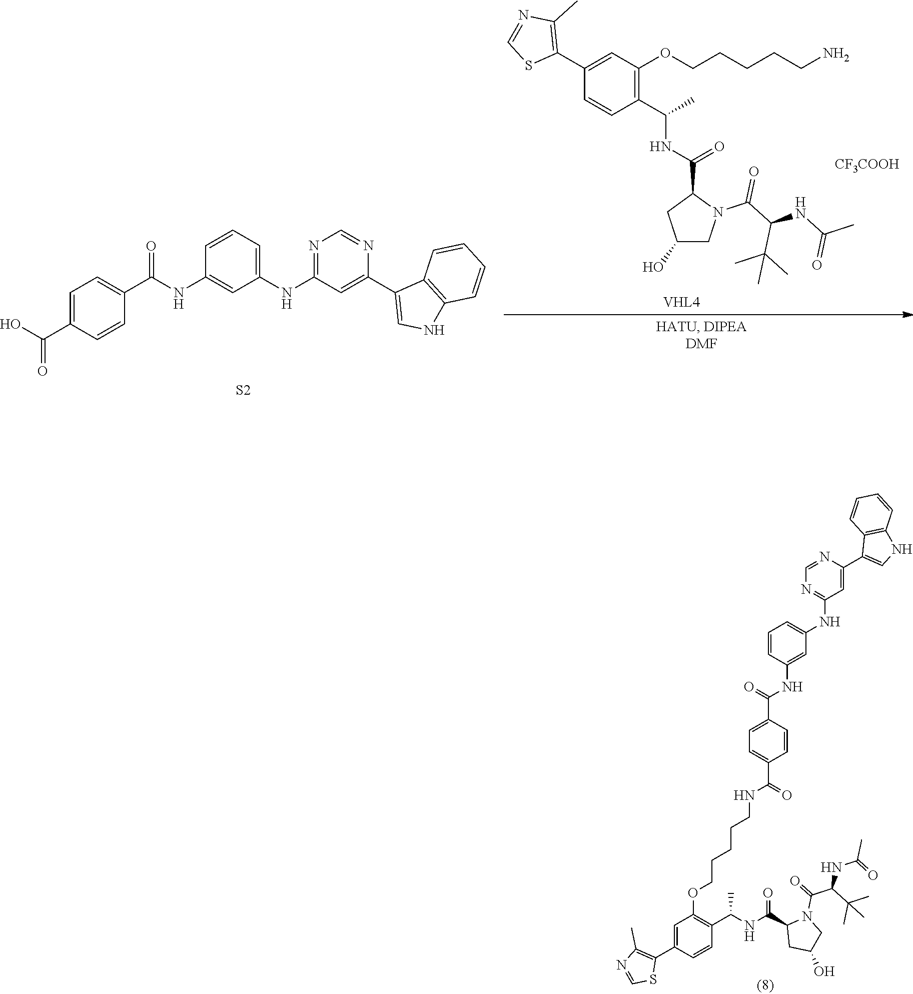 [SMILES: O=C(O)c1ccc(C(=O)Nc2cccc(Nc3cc(-c4c[nH]c5ccccc45)ncn3)c2)cc1 ; CC(=O)N[C@H](C(=O)N1C[C@H](O)C[C@H]1C(=O)N[C@@H](C)c1ccc(-c2scnc2C)cc1OCCCCCN)C(C)(C)C ; "CF3COOH" ; "S2" ; CC(=O)N[C@H](C(=O)N1C[C@H](O)C[C@H]1C(=O)N[C@@H](C)c1ccc(-c2scnc2C)cc1OCCCCCNC(=O)c1ccc(C(=O)Nc2cccc(Nc3cc(-c4c[nH]c5ccccc45)ncn3)c2)cc1)C(C)(C)C]

To a mixture of S2 (9.0 mg, 1.0 eq.), VHL4 (14 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 8 (10.3 mg, 51%) as a white solid.

LC-MS (ESI) m/z: 1019 [M+1]+.

¹H NMR (500 MHZ, DMSO-d₆) δ 12.28 (s, 1H), 10.82 (s, 1H), 10.51 (s, 1H), 8.98 (d, J=3.4 Hz, 1H), 8.87 (s, 1H), 8.65 (t, J=5.7 Hz, 1H), 8.41 (d, J=7.3 Hz, 1H), 8.36 (d, J=3.1 Hz, 1H), 8.32-8.25 (m, 1H), 8.09-7.85 (m, 6H), 7.66-7.37 (m, 6H), 7.36-7.24 (m, 2H), 7.05-6.94 (m, 1H), 6.89 (dd, J=7.9, 1.6 Hz, 1H), 4.98 (t, J=7.0 Hz, 1H), 4.58-4.40 (m, 3H), 4.35 (t, J=3.9 Hz, 2H), 4.07 (t, J=6.3 Hz, 3H), 3.64-3.60 (m, 5H), 3.34 (d, J=6.5 Hz, 3H), 2.47 (d, J=7.0 Hz, 3H), 2.00 (dd, J=8.1, 2.7 Hz, 1H), 1.95-1.77 (m, 6H), 1.65 (q, J=7.0 Hz, 2H), 1.56 (q, J=8.1, 7.7 Hz, 2H), 1.29 (dd, J=16.1, 6.9 Hz, 3H), 0.93 (d, J=2.6 Hz, 2H), 0.85 (s, 9H).

### Example 6: Synthesis of N1-(3-((6-(1H-indol-3-yl)pyrimidin-4-yl)amino)phenyl)-N4-(7-(((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)terephthalamide (9)

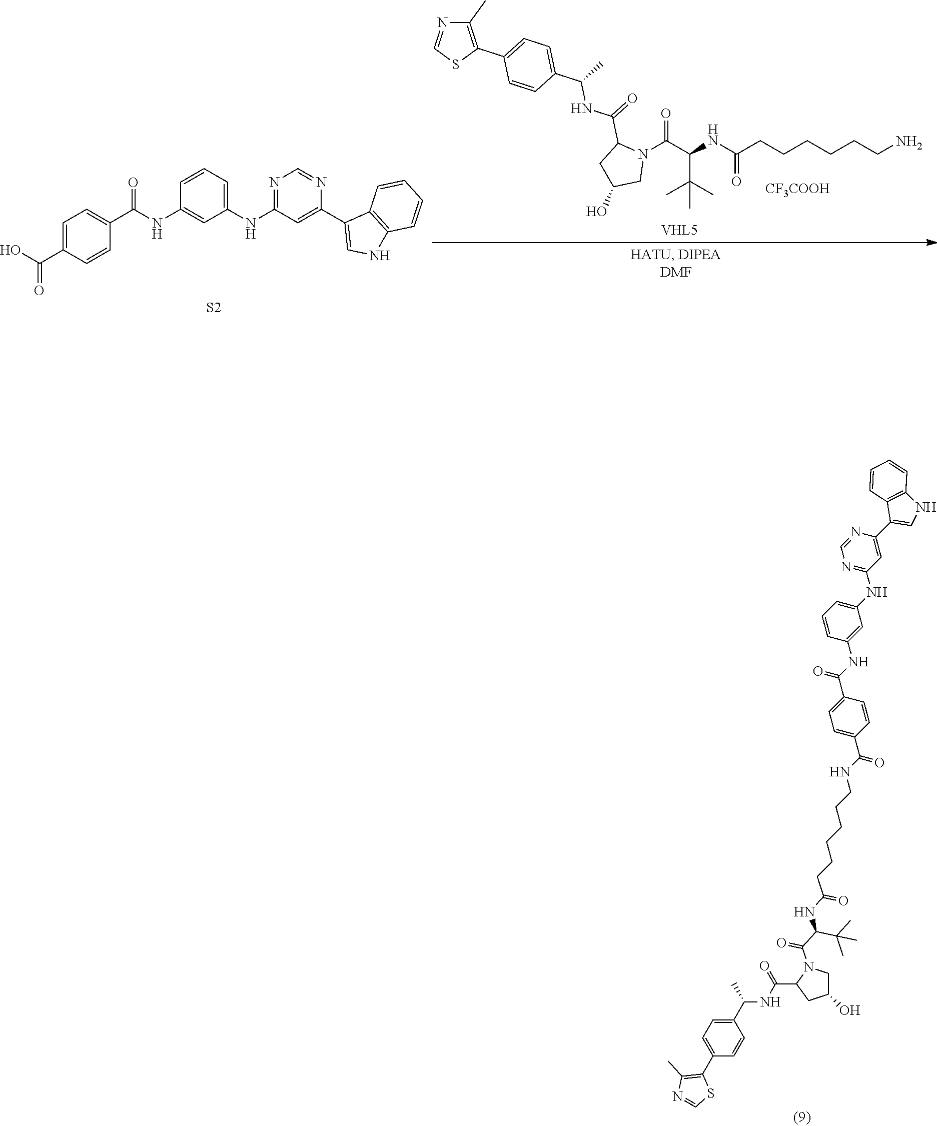 [SMILES: O=C(O)c1ccc(C(=O)Nc2cccc(Nc3cc(-c4c[nH]c5ccccc45)ncn3)c2)cc1 ; Cc1ncsc1-c1ccc([C@H](C)NC(=O)C2C[C@@H](O)CN2C(=O)[C@@H](NC(=O)CCCCCCN)C(C)(C)C)cc1 ; "CF3COOH" ; "S2" ; Cc1ncsc1-c1ccc([C@H](C)NC(=O)C2C[C@@H](O)CN2C(=O)[C@@H](NC(=O)CCCCCCNC(=O)c2ccc(C(=O)Nc3cccc(Nc4cc(-c5c[nH]c6ccccc56)ncn4)c3)cc2)C(C)(C)C)cc1]

To a mixture of S2 (9.0 mg, 1.0 eq.), VHL5 (14 mg, 1.0 eq.), and DIPEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 9 (9.2 mg, 46%) as a white solid.

LC-MS (ESI) m/z: 1003 [M+1]+.

¹H NMR (500 MHZ, DMSO-d₆) δ 12.30 (s, 1H), 10.86 (s, 1H), 10.51 (d, J=6.0 Hz, 1H), 8.99 (s, 1H), 8.87 (s, 1H), 8.62 (t, J=5.7 Hz, 1H), 8.41-8.33 (m, 2H), 8.30 (d, J=2.2 Hz, 1H), 8.09-7.95 (m, 5H), 7.80 (d, J=9.3 Hz, 1H), 7.64-7.48 (m, 3H), 7.49-7.40 (m, 5H), 7.38 (d, J=8.2 Hz, 2H), 7.36-7.25 (m, 3H), 4.92 (t, J=7.2 Hz, 2H), 4.60-4.41 (m, 3H), 4.29 (t, J=3.7 Hz, 2H), 3.28 (q, J=6.9 Hz, 4H), 2.46 (s, 4H), 2.32-2.20 (m, 2H), 2.14 (dt, J=14.4, 7.3 Hz, 2H), 2.01 (td, J=9.1, 7.7, 4.4 Hz, 1H), 1.80 (ddd, J=12.9, 8.5, 4.6 Hz, 1H), 1.53 (dp, J=14.1, 6.7 Hz, 6H), 1.34 (dd, J=32.8, 6.6 Hz, 9H), 0.94 (d, J=6.5 Hz, 9H).

### Example 7: Synthesis of N-(3-((6-(1H-indol-3-yl)pyrimidin-4-yl)amino)phenyl)-4-(10-(2-((2-(2,6-dioxopiperidin-3-yl)-1,3-dioxoisoindolin-4-yl)oxy)acetamido)decanamido)benzamide (13)

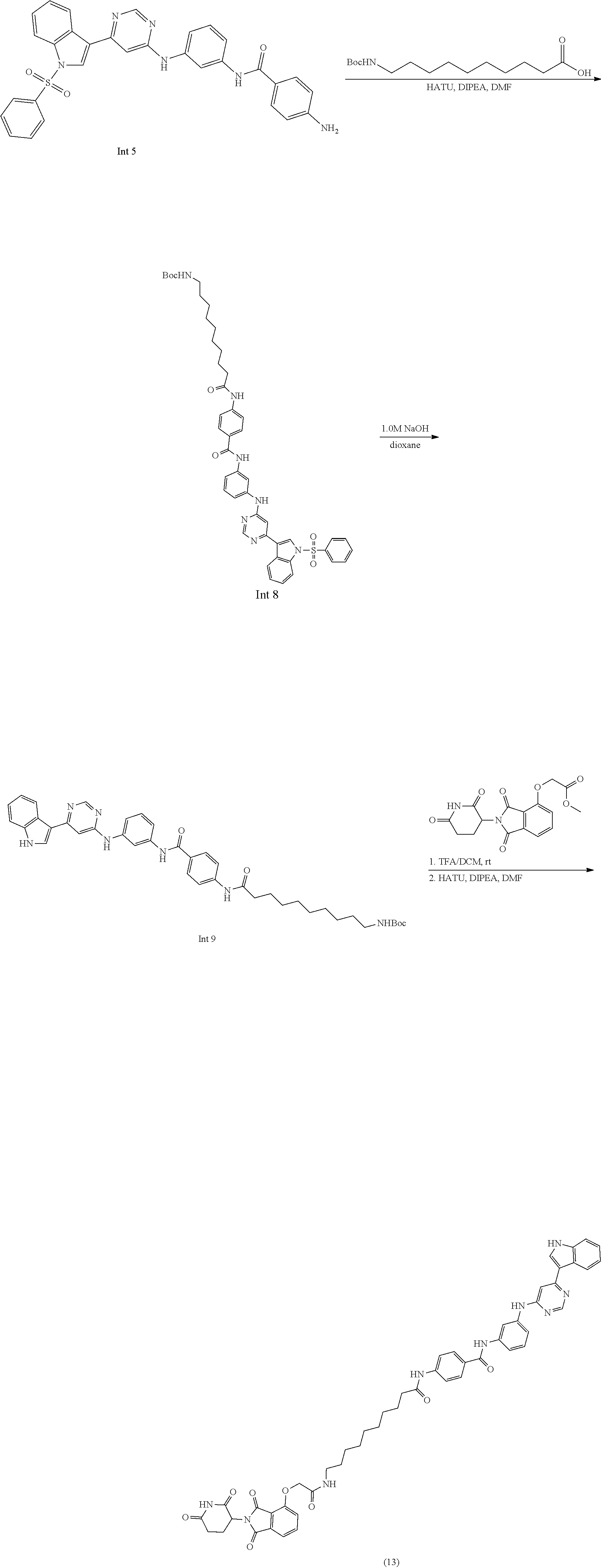 [SMILES: Nc1ccc(C(=O)Nc2cccc(Nc3cc(-c4cn(S(=O)(=O)c5ccccc5)c5ccccc45)ncn3)c2)cc1 ; CC(C)(C)OC(=O)NCCCCCCCCCC(=O)O ; CC(C)(C)OC(=O)NCCCCCCCCCC(=O)Nc1ccc(C(=O)Nc2cccc(Nc3cc(-c4cn(S(=O)(=O)c5ccccc5)c5ccccc45)ncn3)c2)cc1 ; CC(C)(C)OC(=O)NCCCCCCCCCC(=O)Nc1ccc(C(=O)Nc2cccc(Nc3cc(-c4c[nH]c5ccccc45)ncn3)c2)cc1 ; COC(=O)COc1cccc2c1C(=O)N(C1CCC(=O)NC1=O)C2=O ; O=C(COc1cccc2c1C(=O)N(C1CCC(=O)NC1=O)C2=O)NCCCCCCCCCC(=O)Nc1ccc(C(=O)Nc2cccc(Nc3cc(-c4c[nH]c5ccccc45)ncn3)c2)cc1]

To a solution of Int 5 (28 mg, 0.05 mmol, 1 eq.) in DMF (1 mL) was added 10-((tert-butoxycarbonyl)amino) decanoic acid (17 mg, 1.2 eq.), HATU (38 mg, 2 eq.), and DIEA (32 mg, 5 eq.), and the reaction mixture was stirred at rt overnight. The crude reaction mixture was purified via prep-HPLC to yield Int 8 (33 mg, 80%) as a yellow solid.

LC-MS (ESI) m/z: 830 [M+1]+.

To a solution of Int 8 (33 mg, 0.04 mmol, 1 eq.) in 1,4-dioxane (0.5 mL) was added 1.0 N NaOH (0.5 mL), and the mixture was stirred at rt overnight. The crude reaction mixture was purified via prep-HPLC to Int 9 (21 mg, 76%) as a light-brown solid.

LC-MS (ESI) m/z: 690 [M+1]+.

A mixture of Int 9 (21 mg, 0.03 mmol, 1 eq.) and methyl 2-((2-(2,6-dioxopiperidin-3-yl)-1,3-dioxoisoindolin-4-yl)oxy) acetate (14 mg, 1.2 eq.) was dissolved in 1:1 TFA/DCM (1 mL), and the reaction mixture was stirred at rt for 1.5 h. The solvent was removed under reduced pressure, and the resulting crude product was used in the next step without further purification.

To a solution of the crude product in DMF was added HATU (35 mg, 2 eq.) and DIEA (30 mg, 5 eq.), and the reaction mixture was stirred at rt overnight. The crude reaction mixture was purified via prep-HPLC to yield bifunctional compound 13 (13 mg, 51%) as a light yellow solid.

LC-MS (ESI) m/z 904.40 [M+H]+.

¹H NMR (500 MHZ, DMSO-d₆) δ 12.13 (s, 1H), 11.12 (s, 1H), 10.48 (s, 1H), 10.20 (d, J=35.8 Hz, 2H), 8.80 (s, 1H), 8.37-8.18 (m, 2H), 8.09 (d, J=7.7 Hz, 1H), 7.95 (t, J=8.3 Hz, 3H), 7.85-7.78 (m, 1H), 7.75 (d, J=8.7 Hz, 2H), 7.58 (d, J=7.8 Hz, 1H), 7.52 (dd, J=15.5, 7.5 Hz, 2H), 7.45 (d, J=8.2 Hz, 1H), 7.42-7.35 (m, 3H), 7.28 (p, J=7.1 Hz, 2H), 5.12 (dd, J=12.7, 5.4 Hz, 1H), 4.77 (s, 2H), 3.14 (q, J=6.7 Hz, 3H), 2.90 (ddd, J=16.9, 13.7, 5.5 Hz, 2H), 2.64-2.53 (m, 2H), 2.35 (t, J=7.5 Hz, 2H), 2.10-1.99 (m, 1H), 1.60 (t, J=7.2 Hz, 2H), 1.42 (hept, J=7.7, 7.3 Hz, 3H), 1.28 (d, J=12.2 Hz, 10H).

### Example 8: Synthesis of N-(3-((6-(1H-indol-3-yl)pyrimidin-4-yl)amino)phenyl)-10-(2-((2-(2,6-dioxopiperidin-3-yl)-1,3-dioxoisoindolin-4-yl)oxy)acetamido)decanamide (14)

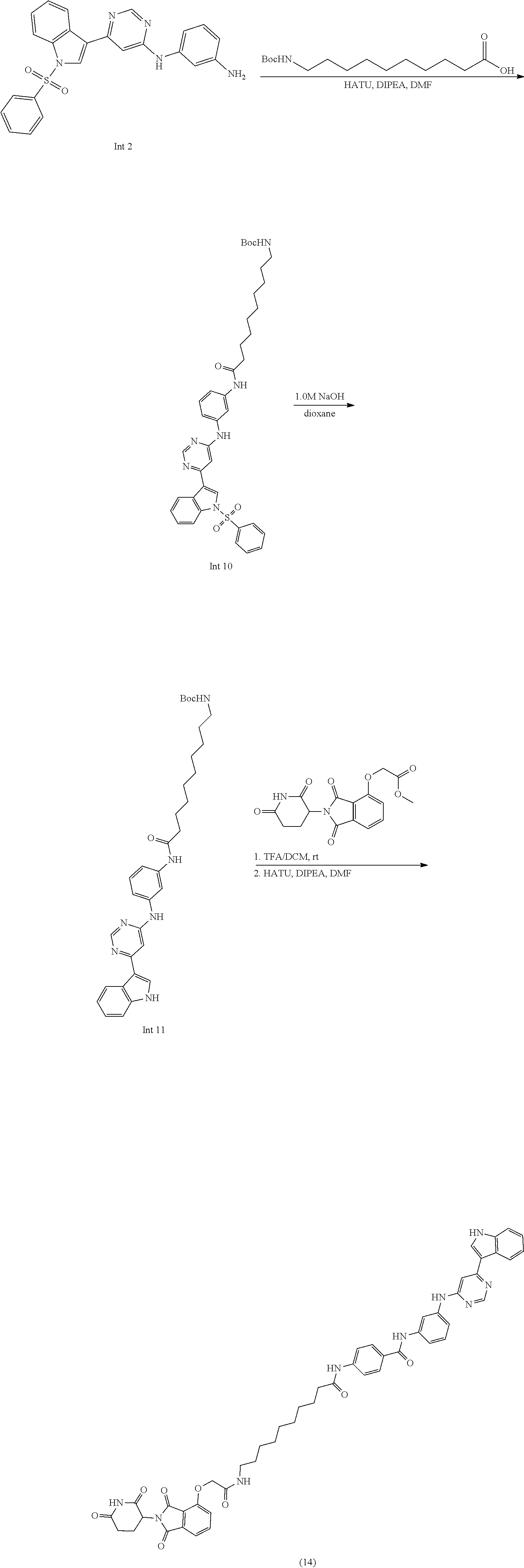 [SMILES: Nc1cccc(Nc2cc(-c3cn(S(=O)(=O)c4ccccc4)c4ccccc34)ncn2)c1 ; CC(C)(C)OC(=O)NCCCCCCCCCC(=O)O ; CC(C)(C)OC(=O)NCCCCCCCCCC(=O)Nc1cccc(Nc2cc(-c3cn(S(=O)(=O)c4ccccc4)c4ccccc34)ncn2)c1 ; CC(C)(C)OC(=O)NCCCCCCCCCC(=O)Nc1cccc(Nc2cc(-c3c[nH]c4ccccc34)ncn2)c1 ; COC(=O)COc1cccc2c1C(=O)N(C1CCC(=O)NC1=O)C2=O ; O=C(COc1cccc2c1C(=O)N(C1CCC(=O)NC1=O)C2=O)NCCCCCCCCCC(=O)Nc1ccc(C(=O)Nc2cccc(Nc3cc(-c4c[nH]c5ccccc45)ncn3)c2)cc1]

To a solution of Int 2 (44 mg, 0.1 mmol, 1 eq.) in DMF (1 mL) was added 10-((tert-butoxycarbonyl)amino) decanoic acid (34 mg, 1.2 eq.), HATU (76 mg, 2 eq.), and DIEA (65 mg, 5 eq.), and the reaction mixture was stirred at rt overnight. The resulting crude mixture was purified via prep-HPLC to yield Int 10 (52 mg, 74%) as a yellow-orange solid.

LC-MS (ESI) m/z: 711 [M+1]+.

To a solution of Int 10 (52 mg, 0.074 mmol, 1 eq.) in 1,4-dioxane (0.5 mL) was added 1.0 N NaOH (0.5 mL), and the mixture was stirred at rt overnight. The crude reaction mixture was purified via prep-HPLC to yield Int 11 (36 mg, 88%) as a light-yellow solid.

LC-MS (ESI) m/z: 571 [M+1]+.

Int 11 (18 mg, 0.027 mmol, 1.2 eq.) was dissolved in 1:1 TFA/DCM (1 mL), and the reaction mixture was stirred at rt for 1.5 h. The solvent was removed under reduced pressure, and the crude residue was used in the next step without further purification.

To a solution of the crude residue in DMF was added HATU (17 mg, 2 eq.) and DIEA (15 mg, 5 eq.), and the reaction mixture was stirred at rt overnight. The crude mixture was purified via prep-HPLC to yield bifunctional compound 14 (8 mg, 45%) as a light yellow solid.

LC-MS (ESI) m/z 785.44 [M+H]+.

¹H NMR (500 MHZ, DMSO-d₆) δ 12.16 (s, 1H), 11.12 (s, 1H), 10.50 (s, 1H), 9.99 (s, 1H), 8.80 (s, 1H), 8.30 (d, J=3.1 Hz, 1H), 8.11-8.02 (m, 2H), 7.93 (t, J=5.7 Hz, 1H), 7.81 (dd, J=8.5, 7.3 Hz, 1H), 7.62-7.55 (m, 1H), 7.50 (d, J=7.3 Hz, 2H), 7.42-7.23 (m, 5H), 5.12 (dd, J=12.8, 5.5 Hz, 1H), 4.77 (s, 2H), 3.14 (q, J=6.6 Hz, 2H), 2.90 (ddd, J=16.8, 13.7, 5.4 Hz, 1H), 2.63-2.53 (m, 1H), 2.33 (t, J=7.4 Hz, 2H), 2.09-1.96 (m, 1H), 1.60 (t, J=7.2 Hz, 2H), 1.43 (t, J=6.9 Hz, 2H), 1.35-1.18 (d, J=18.7 Hz, 10H).

### Example 9: Synthesis of N-(3-((6-(1H-indol-3-yl)pyrimidin-4-yl)amino)phenyl)-10-((2-(2,6-dioxopiperidin-3-yl)-1,3-dioxoisoindolin-4-yl)amino)decanamide (15)

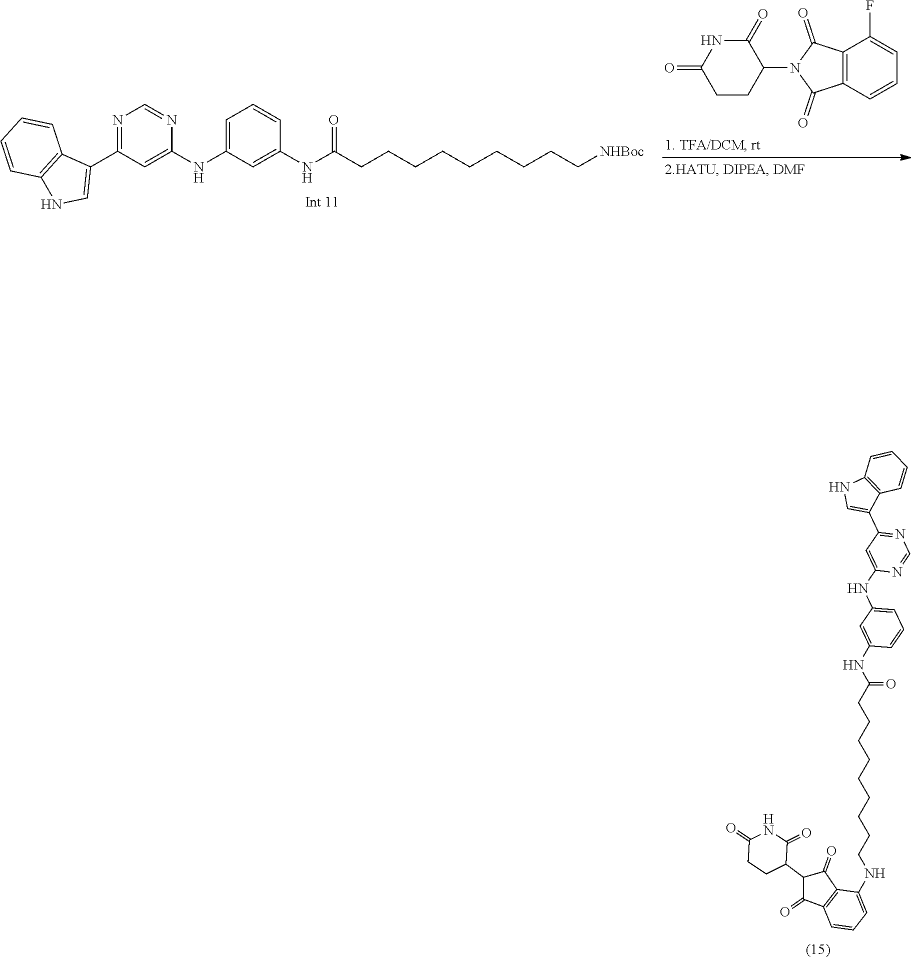 [SMILES: CC(C)(C)OC(=O)NCCCCCCCCCC(=O)Nc1cccc(Nc2cc(-c3c[nH]c4ccccc34)ncn2)c1 ; O=C1CCC(N2C(=O)c3cccc(F)c3C2=O)C(=O)N1 ; O=C1CCC(C2C(=O)c3cccc(NCCCCCCCCCC(=O)Nc4cccc(Nc5cc(-c6c[nH]c7ccccc67)ncn5)c4)c3C2=O)C(=O)N1]

A mixture of Int 11 (18 mg, 0.027 mmol, 1 eq.) was dissolved in 1:1 TFA/DCM (1 mL), and the reaction was stirred at rt for 1.5 h. The solvent was removed under reduced pressure, and the crude residue was used in the next step without further purification.

To a solution of the crude residue in DMF (0.5 mL) was added 2-(2,6-dioxopiperidin-3-yl)-4-fluoroisoindoline-1,3-dione (9 mg, 1 eq.) and DIEA (10 mg, 3 eq.), and the reaction mixture was stirred at 90° C. overnight. The crude mixture was purified via prep-HPLC to yield bifunctional compound 15 (7.6 mg, 39%) as a yellow solid.

LC-MS (ESI) m/z 727.46 [M+H]+.

1H NMR (500 MHZ, DMSO-d₆) δ 12.15 (s, 1H), 11.09 (s, 1H), 10.49 (s, 1H), 9.99 (s, 1H), 8.80 (s, 1H), 8.29 (d, J=3.0 Hz, 1H), 8.16-7.98 (m, 2H), 7.61-7.54 (m, 2H), 7.48 (s, 1H), 7.39-7.24 (m, 4H), 7.08 (d, J=8.6 Hz, 1H), 7.01 (d, J=7.0 Hz, 1H), 6.51 (d, J=5.7 Hz, 1H), 5.05 (dd, J=12.8, 5.4 Hz, 1H), 3.28 (d, J=6.5 Hz, 2H), 2.93-2.80 (m, 1H), 2.58 (dt, J=17.1, 3.3 Hz, 1H), 2.33 (t, J=7.4 Hz, 2H), 2.07-1.94 (m, 1H), 1.67-1.48 (m, 4H), 1.40-1.18 (m, 10H).

### Example 10: Synthesis of N-(3-((6-(1H-indol-3-yl)pyrimidin-4-yl)amino)phenyl)-9-(2-((2-(2,6-dioxopiperidin-3-yl)-1,3-dioxoisoindolin-4-yl)oxy)acetamido)nonanamide (16)

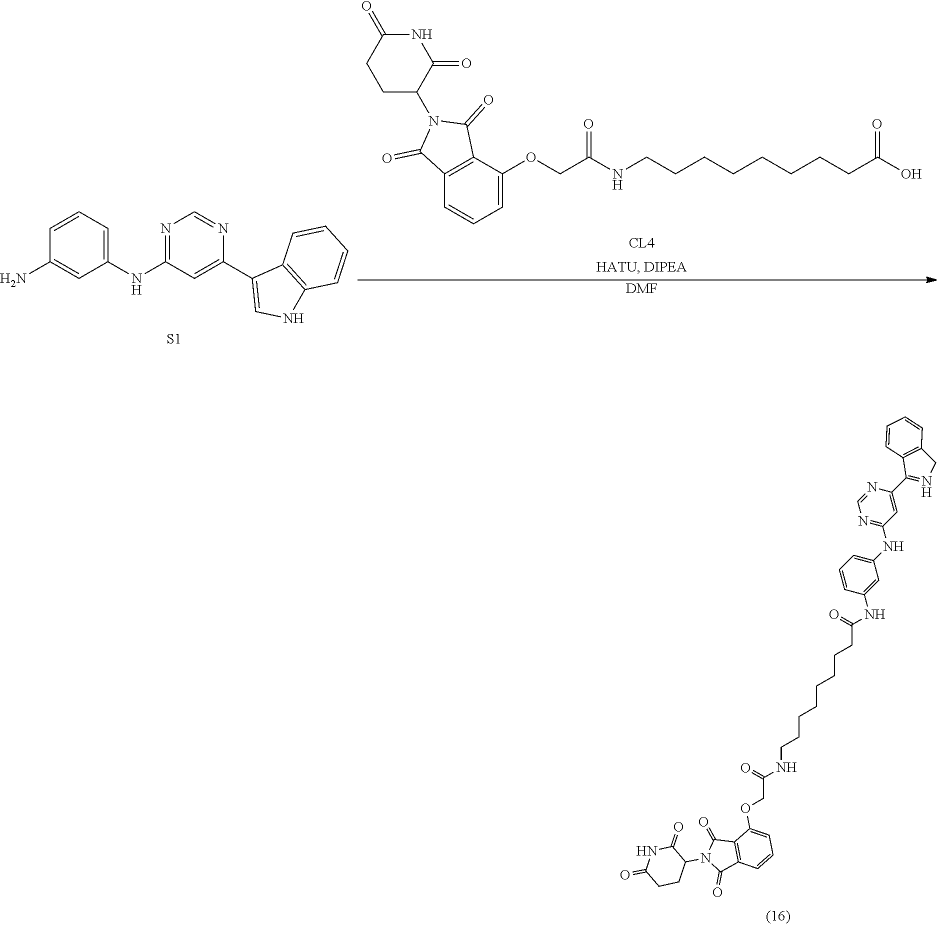

To a mixture of S1 (6.0 mg, 1.0 eq.), CL4 (10 mg, 1.0 eq.), and DIEA (3.0 eq.) in DMF (0.5 mL) was added HATU (7.0 mg, 1.0 eq.), and the resulting solution was stirred at rt overnight. The crude reaction mixture was purified by reverse-phase HPLC to yield bifunctional compound 16 (7.8 mg, 51%) as a white solid.

LC-MS (ESI) m/z: 771 [M+1]+.

1H NMR (500 MHZ, DMSO-d6) δ 12.24 (s, 1H), 11.12 (s, 1H), 10.68 (s, 1H), 10.01 (s, 1H), 8.83 (s, 1H), 8.33 (d, J=3.1 Hz, 1H), 8.09 (d, J=2.2 Hz, 1H), 8.03 (d, J=7.5 Hz, 1H), 7.94 (t, J=5.8 Hz, 1H), 7.84-7.78 (m, 1H), 7.63-7.57 (m, 1H), 7.50 (d, J=7.2 Hz, 2H), 7.42-7.22 (m, 6H), 5.12 (dd, J=12.8, 5.4 Hz, 1H), 4.77 (s, 2H), 3.14 (q, J=6.7 Hz, 2H), 2.98-2.82 (m, 1H), 2.60 (dt, J=17.1, 3.0 Hz, 1H), 2.33 (t, J=7.4 Hz, 2H), 2.09-1.99 (m, 1H), 1.60 (t, J=7.2 Hz, 2H), 1.44 (t, J=6.8 Hz, 2H), 1.36-1.17 (m, 8H).

### Example 11: Synthesis of N-(3-((6-(1H-indol-3-yl)pyrimidin-4-yl)amino)phenyl)-3-(2-(2-(2-((2-(2,6-dioxopiperidin-3-yl)-1,3-dioxoisoindolin-4-yl)oxy)acetamido)ethoxy)ethoxy)propanamide (17)

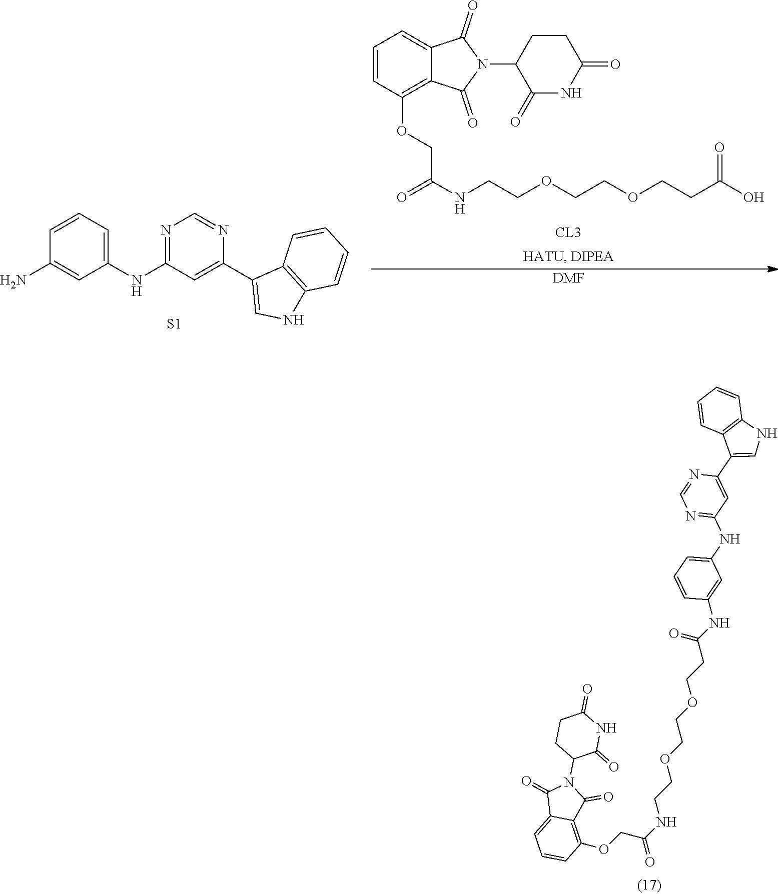

To a mixture of S1 (6.0 mg, 1.0 eq.), CL3 (10 mg, 1.0 eq.), and DIEA (3.0 eq.) in DMF (0.5 mL) was added HATU (7.0 mg, 1.0 eq.), and the resulting solution was stirred at rt overnight. The crude reaction mixture was purified by reverse-phase HPLC to yield bifunctional compound 17 (5.8 mg, 38%) as a white solid.

LC-MS (ESI) m/z: 775 [M+1]+.

¹H NMR (500 MHZ, DMSO-d₆) δ 12.24 (s, 1H), 11.12 (s, 1H), 10.68 (s, 1H), 10.09 (s, 1H), 8.83 (s, 1H), 8.33 (d, J=3.1 Hz, 1H), 8.09 (d, J=2.1 Hz, 1H), 8.07-7.95 (m, 2H), 7.79 (dd, J=8.5, 7.3 Hz, 1H), 7.65-7.57 (m, 1H), 7.48 (d, J=7.2 Hz, 2H), 7.43-7.25 (m, 6H), 5.11 (dd, J=12.8, 5.5 Hz, 1H), 4.78 (s, 2H), 3.56-3.52 (m, 8H), 3.47 (t, J=5.7 Hz, 2H), 3.30 (d, J=5.7 Hz, 2H), 2.97-2.84 (m, 1H), 2.74 (s, 1H), 2.59 (t, J=6.3 Hz, 2H), 2.09-1.96 (m, 2H).

### Example 12: Synthesis of N-(3-((6-(1H-indol-3-yl)pyrimidin-4-yl)amino)phenyl)-3-(2-(2-(2-(2-((2-(2,6-dioxopiperidin-3-yl)-1,3-dioxoisoindolin-4-yl)oxy)acetamido)ethoxy)ethoxy)ethoxy)propanamide (18)

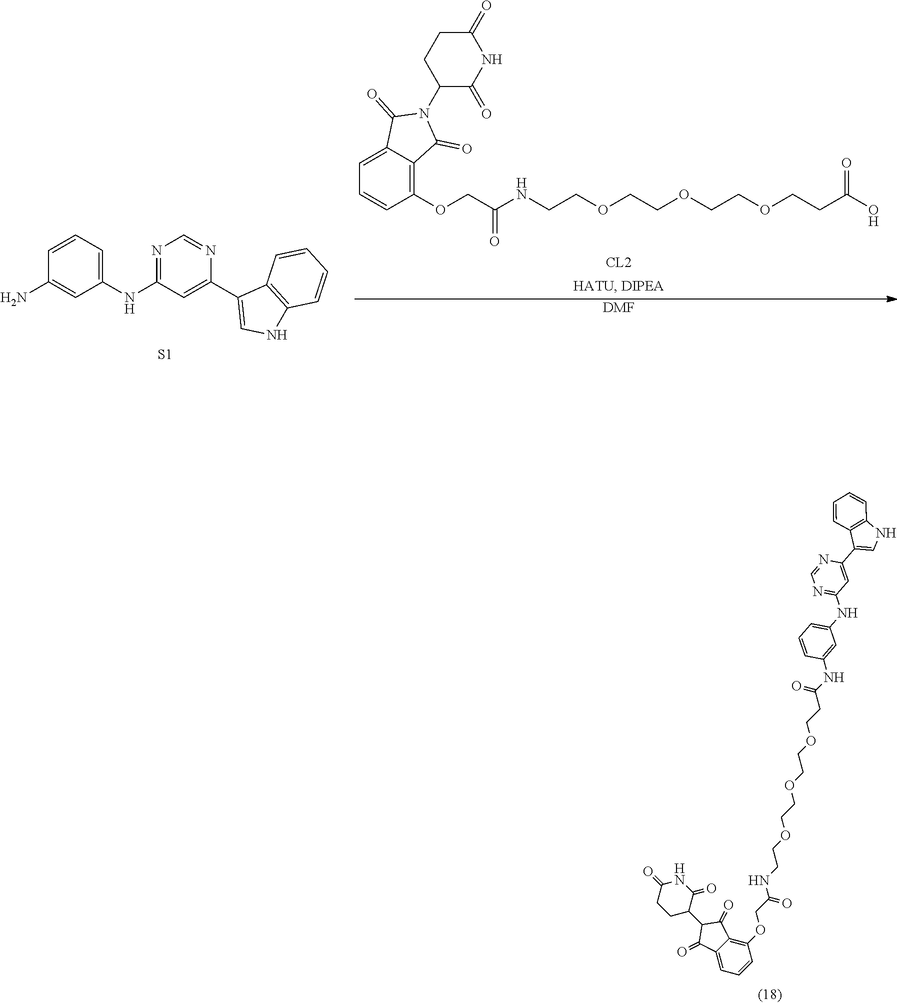

To a mixture of S1 (6.0 mg, 1.0 eq.), CL2 (11 mg, 1.0 eq.), and DIEA (3.0 eq.) in DMF (0.5 mL) was added HATU (7.0 mg, 1.0 eq), and the resulting mixture was stirred at rt overnight. The crude reaction mixture was purified by reverse-phase HPLC to yield bifunctional compound 18 (7.3 mg, 45%) as a white solid.

LC-MS (ESI) m/z: 819 [M+1]+.

1H NMR (500 MHz, DMSO-d₆) δ 12.25 (s, 1H), 11.12 (s, 1H), 10.69 (s, 1H), 10.09 (s, 1H), 8.84 (s, 1H), 8.33 (d, J=3.1 Hz, 1H), 8.10 (d, J=2.2 Hz, 1H), 8.06-7.96 (m, 2H), 7.83-7.75 (m, 1H), 7.64-7.56 (m, 1H), 7.49 (d, J=7.3 Hz, 2H), 7.45-7.25 (m, 6H), 5.11 (dd, J=12.8, 5.5 Hz, 1H), 4.78 (s, 2H), 3.58-3.48 (m, 12H), 3.44 (t, J=5.7 Hz, 2H), 3.30 (q, J=5.7 Hz, 2H), 2.89 (ddd, J=17.1, 13.6, 5.4 Hz, 1H), 2.58 (q, J=5.0, 3.8 Hz, 2H), 2.11-1.97 (m, 1H).

### Example 13: Synthesis of N-(3-((6-(1H-indol-3-yl)pyrimidin-4-yl)amino)phenyl)-1-(2-((2-(2,6-dioxopiperidin-3-yl)-1,3-dioxoisoindolin-4-yl)oxy)acetamido)-3,6,9,12-tetraoxapentadecan-15-amide (19)

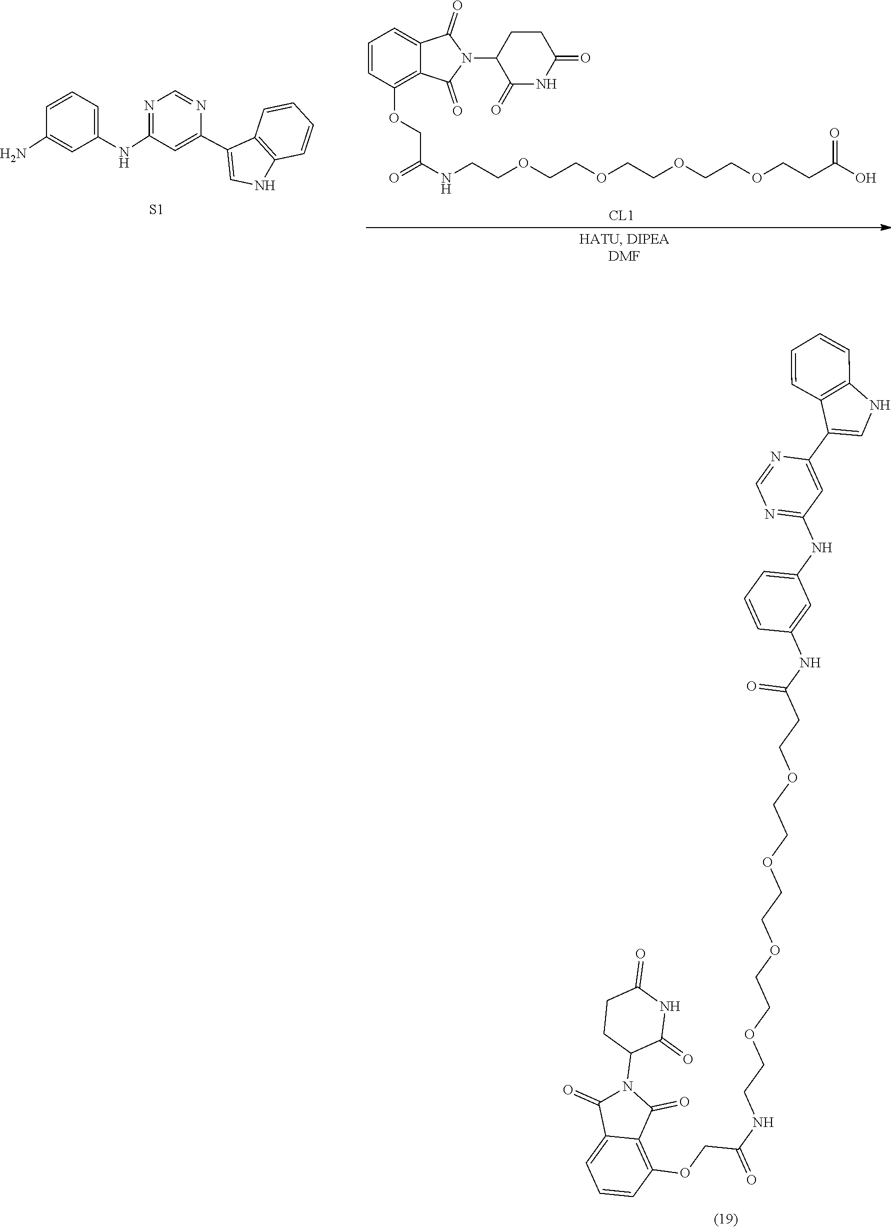 [SMILES: Nc1cccc(Nc2cc(-c3c[nH]c4ccccc34)ncn2)c1 ; O=C(O)CCOCCOCCOCCOCCNC(=O)COc1cccc2c1C(=O)N(C1CCC(=O)NC1=O)C2=O ; "S1" ; O=C(COc1cccc2c1C(=O)N(C1CCC(=O)NC1=O)C2=O)NCCOCCOCCOCCOCCC(=O)Nc1cccc(Nc2cc(-c3c[nH]c4ccccc34)ncn2)c1]

To a mixture of S1 (6.0 mg, 1.0 eq.), CL1 (11 mg, 1.0 eq.), and DIEA (3.0 eq.) in DMF (0.5 mL) was added HATU (8.0 mg, 1.0 eq.), and the reaction mixture was stirred at rt overnight. The resulting crude mixture was purified by reverse-phase HPLC to yield bifunctional compound 19 (7.3 mg, 43%) as a white solid.

LC-MS (ESI) m/z: 863 [M+1]+.

¹H NMR (500 MHZ, DMSO-d₆) δ 12.21 (s, 1H), 11.12 (s, 1H), 10.61 (s, 1H), 10.08 (s, 1H), 8.82 (s, 1H), 8.31 (d, J=3.1 Hz, 1H), 8.10 (d, J=2.2 Hz, 1H), 8.04 (d, J=7.4 Hz, 1H), 8.00 (t, J=5.7 Hz, 1H), 7.80 (dd, J=8.5, 7.3 Hz, 1H), 7.60 (dd, J=6.7, 2.3 Hz, 1H), 7.49 (d, J=7.2 Hz, 2H), 7.42-7.23 (m, 6H), 5.11 (dd, J=12.8, 5.5 Hz, 1H), 4.78 (s, 2H), 3.72 (t, J=6.2 Hz, 2H), 3.54-3.41 (m, 16H), 3.31 (q, J=5.7 Hz, 2H), 2.89 (ddd, J=17.0, 13.8, 5.4 Hz, 1H), 2.59 (t, J=6.2 Hz, 2H), 2.04 (ddd, J=7.4, 5.5, 2.4 Hz, 1H).

### Example 14: Synthesis of N1-(4-((3-((6-(1H-indol-3-yl)pyrimidin-4-yl)amino)phenyl)carbamoyl)phenyl)-N8-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (20)

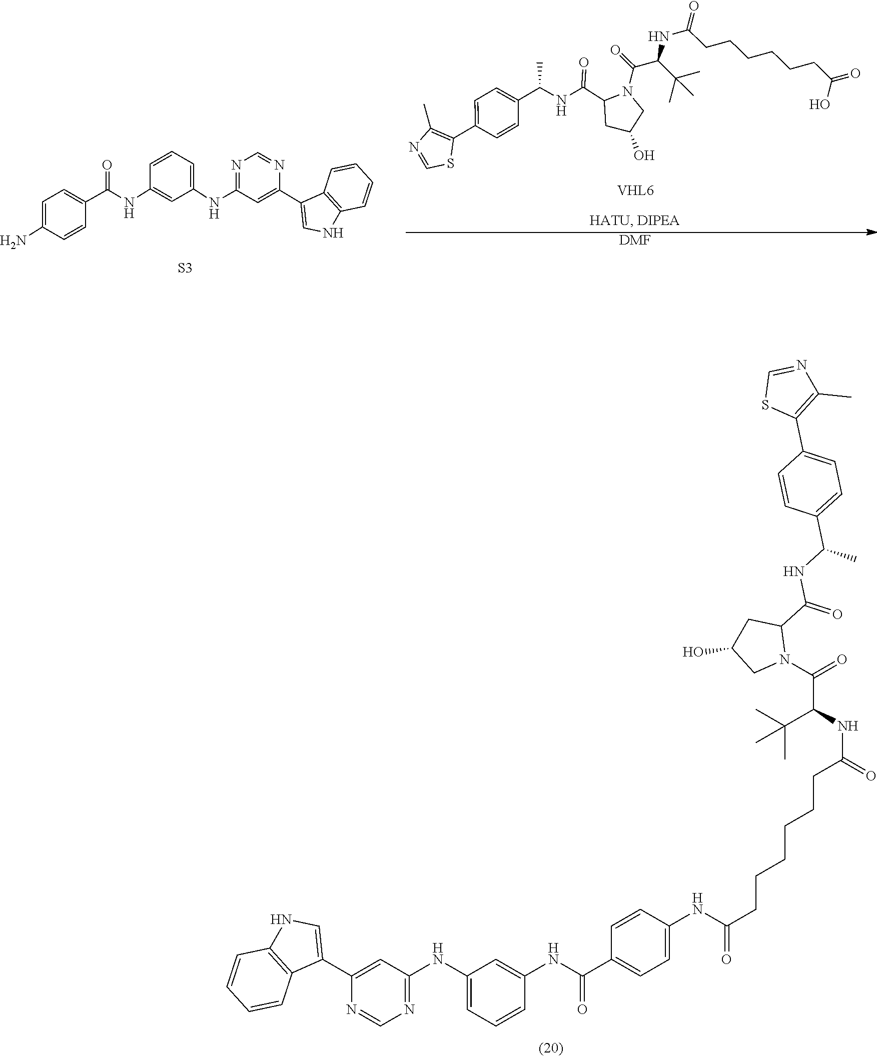

To a mixture of S3 (8.0 mg, 1.0 eq.), VHL6 (12 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 20 (9.1 mg, 45%) as a white solid.

LC-MS (ESI) m/z: 1003 [M+1]+.

¹H NMR (500 MHZ, DMSO-d₆) δ 12.30 (s, 1H), 10.84 (s, 1H), 10.23 (d, J=46.7 Hz, 2H), 8.93 (d, J=59.4 Hz, 2H), 8.41-8.32 (m, 2H), 8.28 (d, J=2.2 Hz, 1H), 8.04 (d, J=7.7 Hz, 1H), 7.99-7.92 (m, 2H), 7.82-7.72 (m, 3H), 7.64-7.58 (m, 1H), 7.57-7.47 (m, 2H), 7.46-7.35 (m, 7H), 7.36-7.27 (m, 3H), 4.99-4.83 (m, 2H), 4.57-4.36 (m, 4H), 4.29 (d, J=5.3 Hz, 2H), 2.46 (s, 4H), 2.35 (t, J=7.4 Hz, 2H), 2.31-2.22 (m, 2H), 2.19-2.08 (m, 2H), 2.01 (d, J=10.2 Hz, 1H), 1.80 (ddd, J=12.9, 8.5, 4.6 Hz, 2H), 1.66-1.43 (m, 6H), 1.38 (d, J=7.0 Hz, 4H), 1.34-1.23 (m, 5H), 0.94 (d, J=2.6 Hz, 9H).

### Example 15: Synthesis of N1-(4-((3-((6-(1H-indol-3-yl)pyrimidin-4-yl)amino)phenyl)carbamoyl)phenyl)-N4-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)succinamide (21)

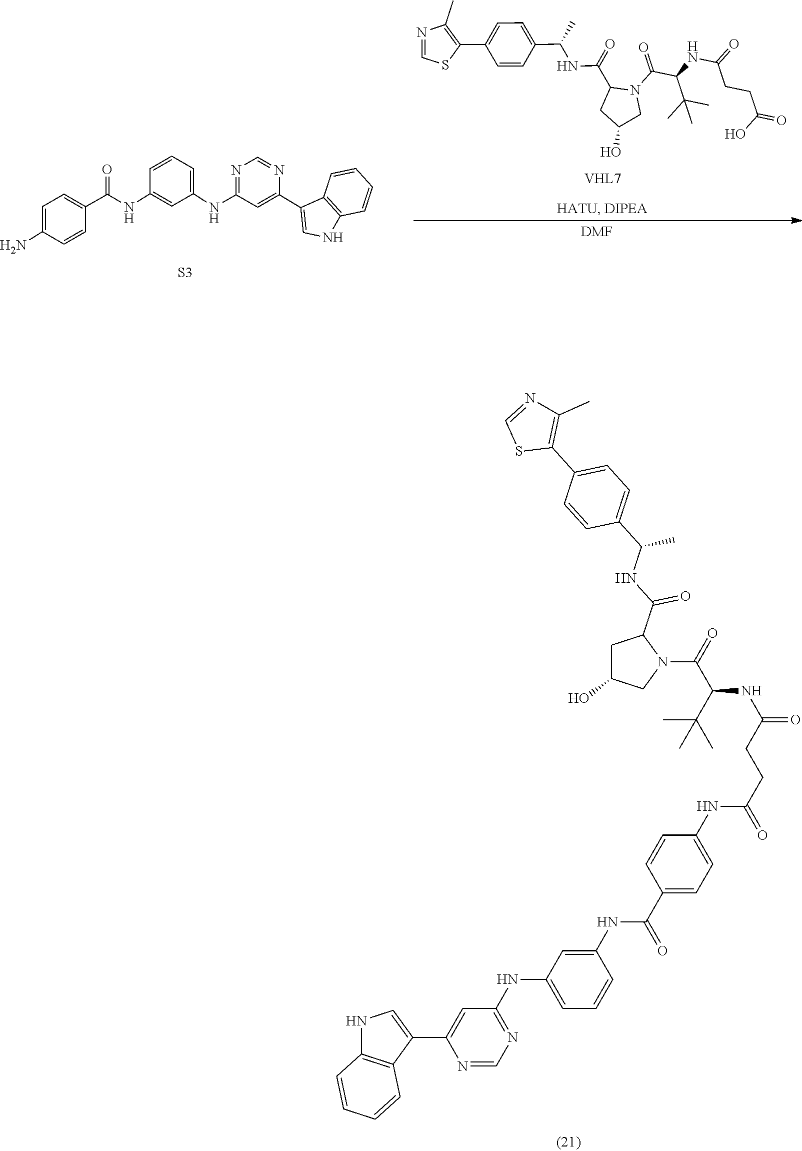

To a mixture of S3 (8.0 mg, 1.0 eq.), VHL7 (12 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 21 (10.6 mg, 56%) as a white solid.

LC-MS (ESI) m/z: 947 [M+1]+.

¹H NMR (500 MHZ, DMSO-d₆) δ 12.34 (s, 1H), 10.95 (s, 1H), 10.29 (s, 1H), 10.26 (s, 1H), 9.00 (s, 1H), 8.89 (s, 1H), 8.44-8.34 (m, 2H), 8.29 (d, J=2.2 Hz, 1H), 8.02 (d, J=7.7 Hz, 1H), 7.95 (dd, J=13.5, 9.0 Hz, 3H), 7.75 (d, J=8.8 Hz, 1H), 7.68-7.58 (m, 1H), 7.51 (d, J=8.3 Hz, 1H), 7.46-7.41 (m, 3H), 7.39 (d, J=8.4 Hz, 2H), 7.31 (td, J=14.4, 6.3 Hz, 2H), 4.96-4.89 (m, 1H), 4.54 (d, J=9.3 Hz, 1H), 4.44 (t, J=8.0 Hz, 1H), 3.66-3.55 (m, 2H), 2.71-2.54 (m, 2H), 2.46 (s, 3H), 2.11-1.94 (m, 1H), 1.80 (ddd, J=12.9, 8.5, 4.7 Hz, 1H), 1.39 (d, J=7.0 Hz, 3H), 0.95 (s, 9H).

### Example 16: Synthesis of N1-(4-((3-((6-(1H-indol-3-yl)pyrimidin-4-yl)amino)phenyl)carbamoyl)phenyl)-N4-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)succinamide (22)

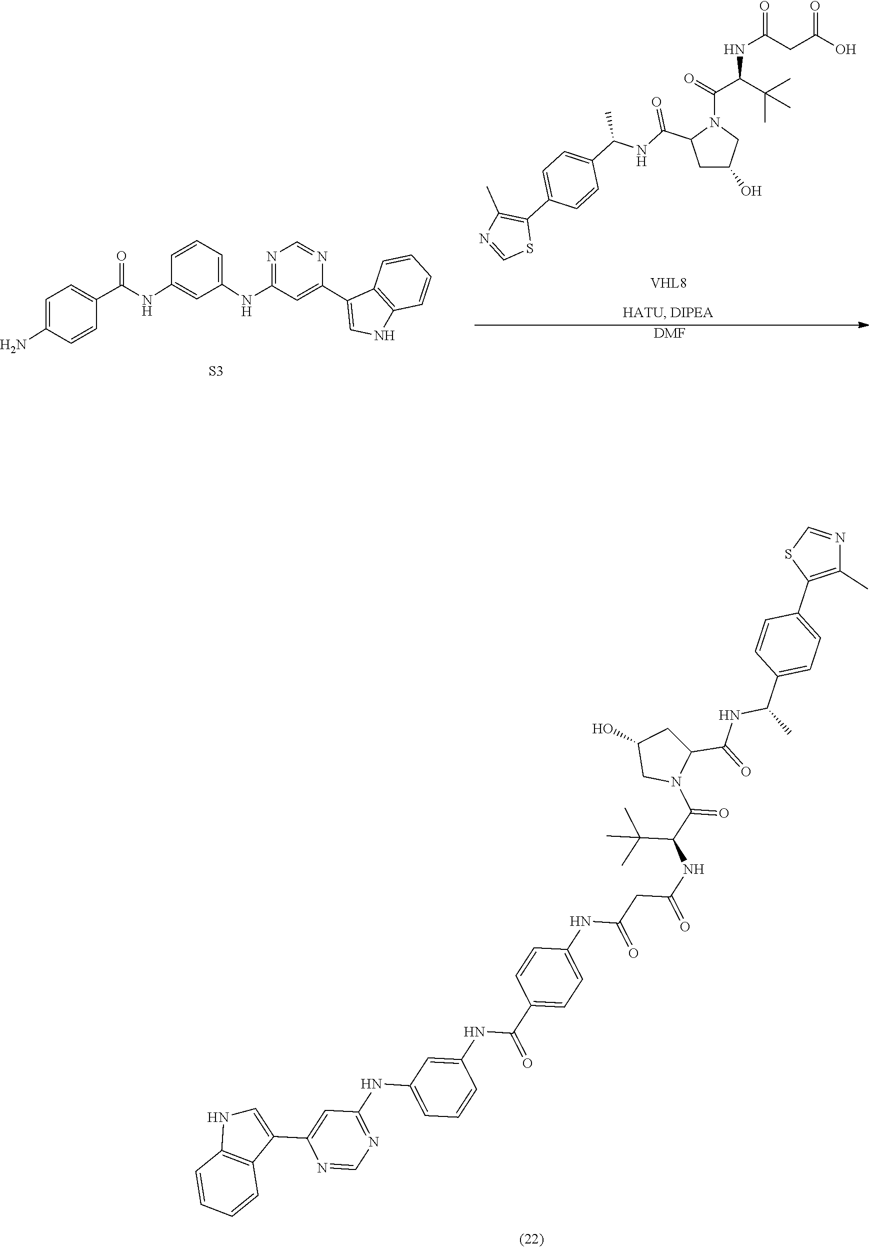

To a mixture of S3 (8.0 mg, 1.0 eq.), VHL8 (11 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 22 (6.2 mg, 33%) as a white solid.

LC-MS (ESI) m/z: 933 [M+1]+.

¹H NMR (500 MHz, DMSO-d₆) δ 12.36 (s, 1H), 10.95 (s, 1H), 10.42 (s, 1H), 10.33 (s, 1H), 8.99 (s, 1H), 8.89 (s, 1H), 8.47-8.35 (m, 2H), 8.31-8.19 (m, 2H), 8.00 (dd, J=20.4, 8.1 Hz, 3H), 7.76 (d, J=8.5 Hz, 2H), 7.62 (d, J=7.8 Hz, 1H), 7.55-7.48 (m, 2H), 7.48-7.37 (m, 6H), 7.36-7.27 (m, 2H), 4.94 (t, J=7.2 Hz, 1H), 4.57 (d, J=9.3 Hz, 1H), 4.46 (t, J=8.1 Hz, 1H), 3.71-3.55 (m, 2H), 3.54-3.28 (m, 2H), 2.46 (s, 3H), 2.10-1.98 (m, 1H), 1.39 (d, J=7.0 Hz, 3H), 0.98 (s, 9H).

### Example 17: Synthesis of N-(3-((6-(1H-indol-3-yl)pyrimidin-4-yl)amino)phenyl)-6-(2-(2,6-dioxopiperidin-3-yl)-1,3-dioxoisoindolin-4-yl)hex-5-ynamide (23)

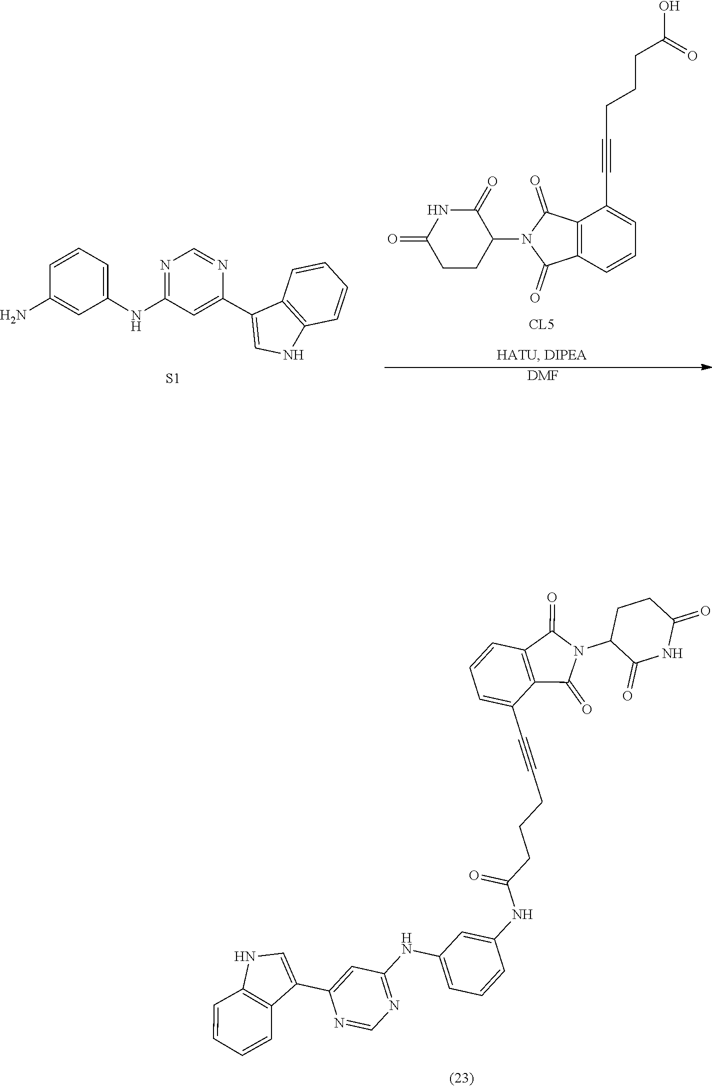

To a mixture of S1 (6.0 mg, 1.0 eq.), CL5 (7.3 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 23 (7.7 mg, 60%) as a white solid.

LC-MS (ESI) m/z: 652 [M+1]+.

¹H NMR (500 MHZ, DMSO-d₆) δ 12.18 (s, 1H), 11.14 (s, 1H), 10.54 (s, 1H), 10.12 (s, 1H), 8.81 (s, 1H), 8.31 (d, J=3.1 Hz, 1H), 8.13-8.01 (m, 2H), 7.87 (hept, J=4.8, 4.2 Hz, 3H), 7.62-7.55 (m, 1H), 7.53-7.43 (m, 1H), 7.41-7.20 (m, 5H), 5.15 (dd, J=12.9, 5.4 Hz, 1H), 2.88 (ddd, J=17.1, 13.8, 5.4 Hz, 1H), 2.59 (dt, J=19.3, 7.1 Hz, 6H), 2.05 (dtd, J=13.0, 5.2, 2.2 Hz, 1H), 1.94 (p, J=7.1 Hz, 2H).

### Example 18: Synthesis of N-(3-((6-(1H-indol-3-yl)pyrimidin-4-yl)amino)phenyl)-8-(2-(2,6-dioxopiperidin-3-yl)-1,3-dioxoisoindolin-4-yl)oct-7-ynamide (24)

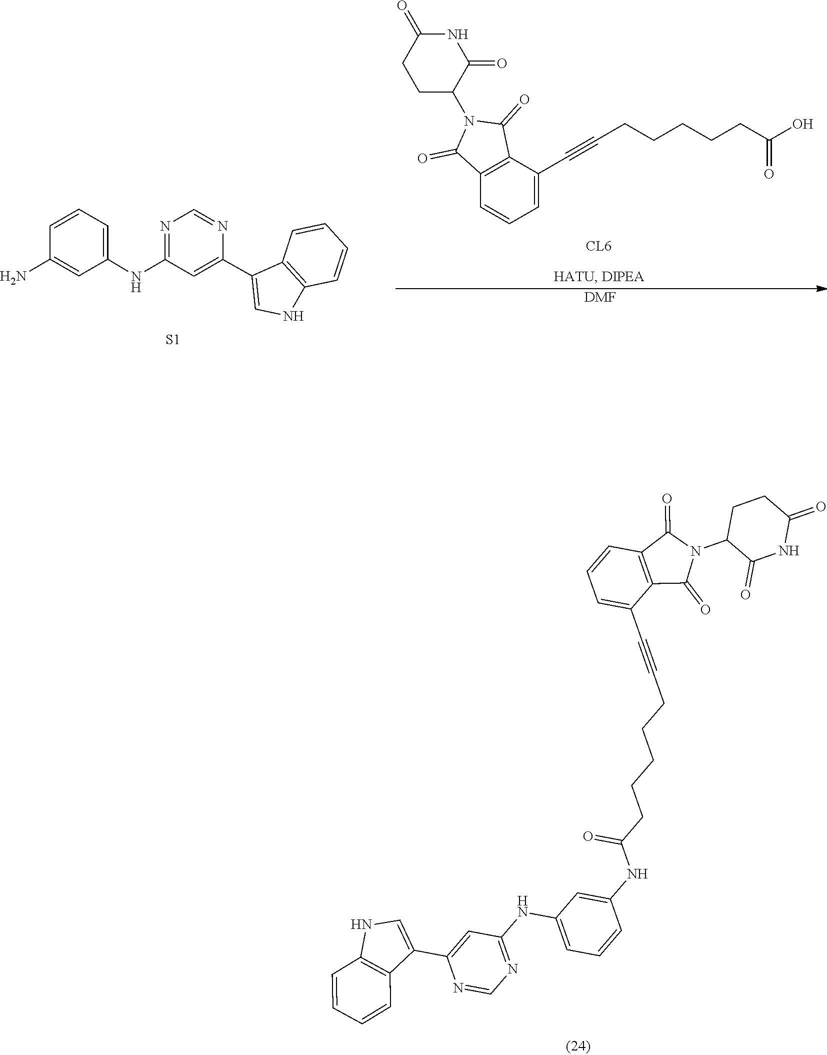

To a mixture of S1 (6.0 mg, 1.0 eq.), CL6 (7.3 mg, 1.0 eq.), and DIEA (5.0 eq.) in DMF (0.5 mL) was added HATU (8.0 mg, 1.0 eq.), and the resulting mixture was stirred at rt overnight. The crude reaction mixture was purified by reverse-phase HPLC to yield bifunctional compound 24 (6.1 mg, 45%) as a white solid.

LC-MS (ESI) m/z: 680 [M+1]+.

¹H NMR (500 MHZ, DMSO-d₆) δ 12.18 (s, 1H), 11.14 (s, 1H), 10.55 (s, 1H), 10.04 (s, 1H), 8.81 (s, 1H), 8.31 (d, J=3.1 Hz, 1H), 8.14-7.96 (m, 2H), 7.91-7.77 (m, 3H), 7.63-7.56 (m, 1H), 7.49 (s, 1H), 7.41-7.24 (m, 5H), 5.15 (dd, J=12.9, 5.4 Hz, 1H), 2.88 (ddd, J=17.1, 13.9, 5.4 Hz, 1H), 2.39 (t, J=7.3 Hz, 2H), 2.09-1.99 (m, 1H), 1.66 (dq, J=24.8, 7.3 Hz, 6H), 1.51 (ddt, J=15.1, 9.8, 5.8 Hz, 2H).

### Example 19: Synthesis of N1-(4-((3-((6-(1H-indol-3-yl)pyrimidin-4-yl)amino)phenyl)carbamoyl)phenyl)-N5-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)glutaramide (25)

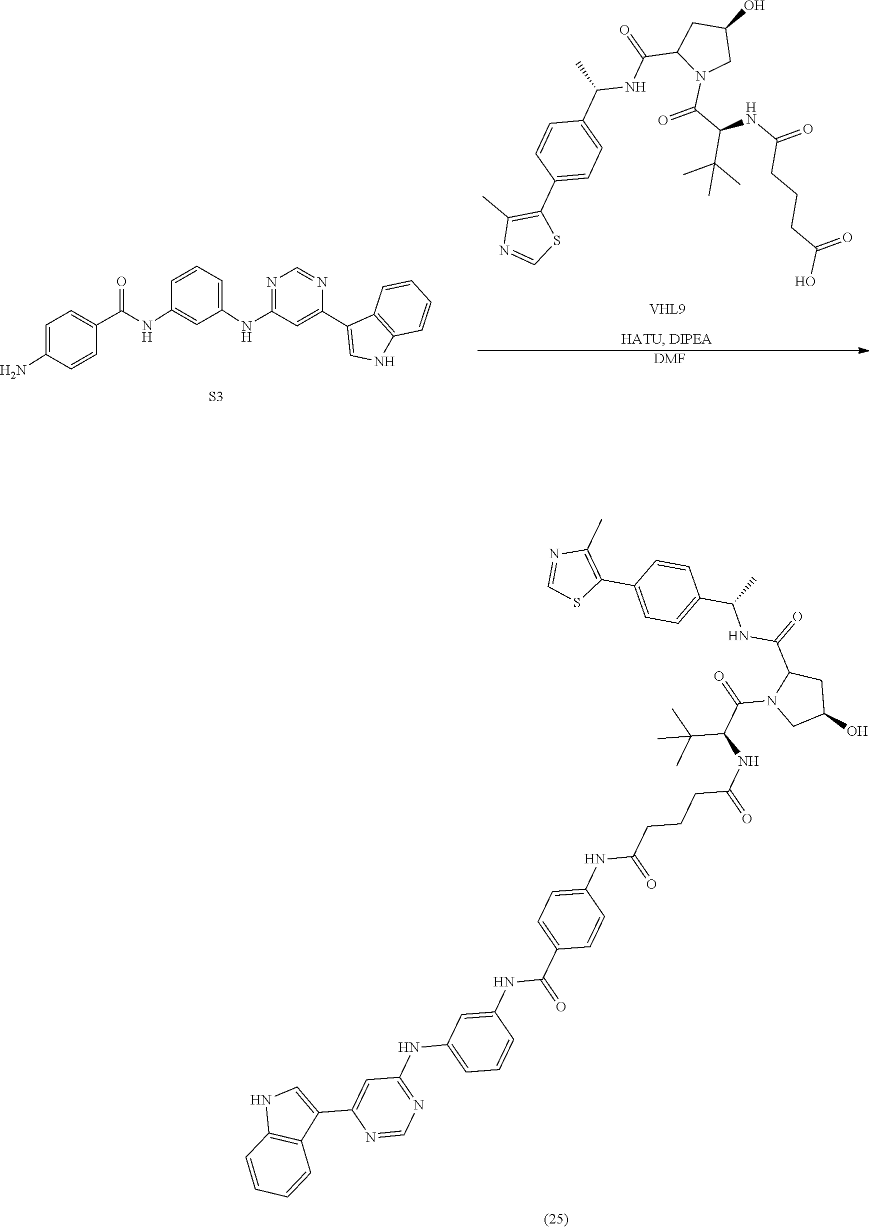

To a mixture of S3 (8.0 mg, 1.0 eq.), VHL9 (11 mg, 1.0 eq.), and DIPEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 25 (7.9 mg, 41%) as a white solid.

LC-MS (ESI) m/z: 961 [M+1]+.

¹H NMR (500 MHZ, DMSO-d₆) δ 12.30 (s, 1H), 10.86 (s, 1H), 10.28 (s, 1H), 10.20 (s, 1H), 8.99 (s, 1H), 8.87 (s, 1H), 8.42-8.35 (m, 2H), 8.29 (d, J=2.2 Hz, 1H), 8.03 (d, J=7.7 Hz, 1H), 7.99-7.91 (m, 2H), 7.88 (d, J=9.2 Hz, 1H), 7.79-7.70 (m, 2H), 7.65-7.58 (m, 1H), 7.58-7.46 (m, 2H), 7.46-7.36 (m, 6H), 7.32 (ddd, J=9.2, 7.5, 5.9 Hz, 2H), 4.93 (t, J=7.2 Hz, 1H), 4.53 (d, J=9.2 Hz, 1H), 4.44 (t, J=8.0 Hz, 1H), 4.30 (s, 1H), 3.63 (d, J=4.2 Hz, 1H), 2.46 (s, 3H), 2.42-2.19 (m, 3H), 2.03 (dd, J=12.5, 9.0 Hz, 1H), 1.89-1.72 (m, 2H), 1.38 (d, J=7.0 Hz, 3H), 0.95 (d, J=8.6 Hz, 9H).

### Example 20: Synthesis of N1-(4-((3-((6-(1H-indol-3-yl)pyrimidin-4-yl)amino)phenyl)carbamoyl)phenyl)-N10-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide (26)

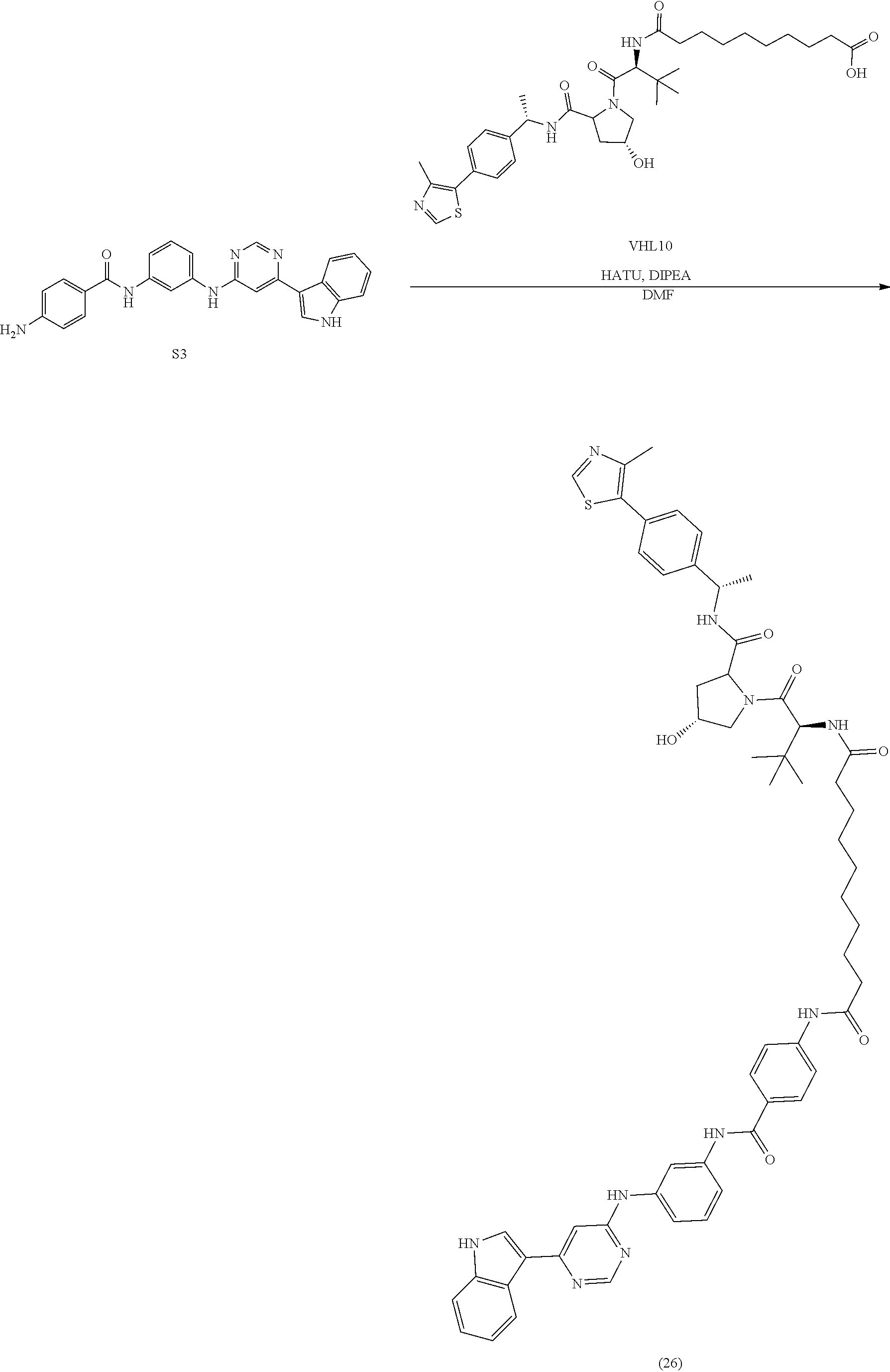

To a mixture of S3 (8.0 mg, 1.0 eq.), VHL10 (14 mg, 1.0 eq.), and DIEA (5.0 eq.) in DMF (0.5 mL) was added HATU (8.0 mg, 1.0 eq.), and the resulting solution was stirred at rt overnight. The crude reaction mixture was purified by reverse-phase HPLC to yield bifunctional compound 26 (6.6 mg, 32%) as a white solid.

LC-MS (ESI) m/z: 1031 [M+1]+.

¹H NMR (500 MHZ, DMSO-d₆) δ 12.29 (s, 1H), 10.82 (s, 1H), 10.28 (s, 1H), 10.18 (s, 1H), 8.98 (s, 1H), 8.86 (s, 1H), 8.43-8.32 (m, 2H), 8.28 (d, J=2.2 Hz, 1H), 8.04 (d, J=7.6 Hz, 1H), 8.01-7.88 (m, 2H), 7.82-7.69 (m, 3H), 7.65-7.57 (m, 1H), 7.57-7.46 (m, 1H), 7.48-7.20 (m, 8H), 4.91 (q, J=7.3 Hz, 1H), 4.52 (d, J=9.3 Hz, 1H), 4.43 (t, J=8.0 Hz, 1H), 4.29 (q, J=4.2, 3.6 Hz, 1H), 3.65-3.45 (m, 1H), 2.46 (s, 3H), 2.35 (t, J=7.4 Hz, 2H), 2.26 (dt, J=14.7, 7.7 Hz, 1H), 2.11 (ddd, J=14.1, 8.0, 6.2 Hz, 1H), 2.06-1.95 (m, 1H), 1.80 (ddd, J=12.9, 8.5, 4.6 Hz, 1H), 1.61 (t, J=7.2 Hz, 2H), 1.56-1.41 (m, 1H), 1.38 (d, J=7.0 Hz, 2H), 1.28 (d, J=13.7 Hz, 10H), 0.93 (d, J=5.8 Hz, 9H).

### Example 21: Synthesis of N1-(4-(3-(6-(1H-indol-3-yl)pyrimidin-4-yl)amino)phenyl)carbamoyl)phenyl)-N5-(5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)glutaramide (27)

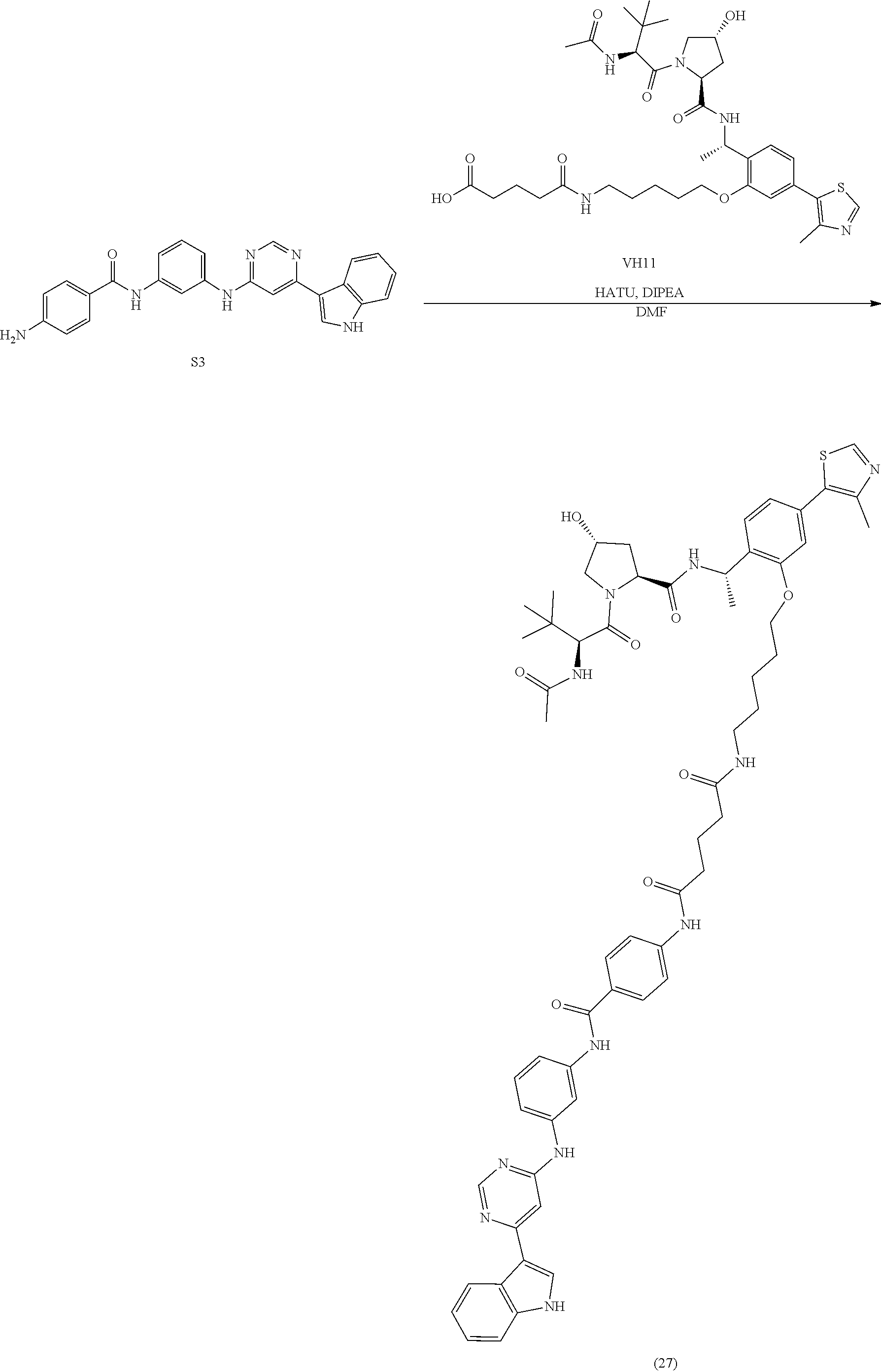

To a mixture of S3 (8.0 mg, 1.0 eq.), VL11 (14 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 27 (9.4 mg, 45%) as white solid.

LC-MS (ESI) m/z: 1104 [M+1]+.

¹H NMR (500 MHZ, DMSO-d₆) δ 12.35 (s, 1H), 10.96 (s, 1H), 10.29 (s, 1H), 10.20 (s, 1H), 8.98 (s, 1H), 8.89 (s, 1H), 8.45-8.34 (m, 2H), 8.29 (t, J=2.1 Hz, 1H), 8.05-7.90 (m, 4H), 7.85-7.71 (m, 3H), 7.66-7.38 (m, 7H), 7.32 (p, J=8.8, 8.0 Hz, 2H), 7.02-6.85 (m, 2H), 4.98 (p, J=7.0 Hz, 1H), 4.58-4.46 (m, 3H), 4.35 (s, 2H), 3.62 (d, J=4.9 Hz, 4H), 3.09 (d, J=6.1 Hz, 3H), 2.46 (s, 3H), 2.36 (t, J=7.5 Hz, 2H), 2.14 (t, J=7.4 Hz, 2H), 2.01 (ddd, J=10.6, 8.1, 4.2 Hz, 1H), 1.89 (d, J=2.8 Hz, 3H), 1.86-1.74 (m, 5H), 1.48 (dt, J=9.3, 4.8 Hz, 5H), 1.32 (t, J=6.2 Hz, 4H), 0.94 (s, 2H), 0.85 (s, 9H).

### Example 22: Synthesis of N1-(3-((6-(1H-indol-3-yl)pyrimidin-4-yl)amino)phenyl)-N4-(3-((5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)amino)-3-oxopropyl)terephthalamide (28)

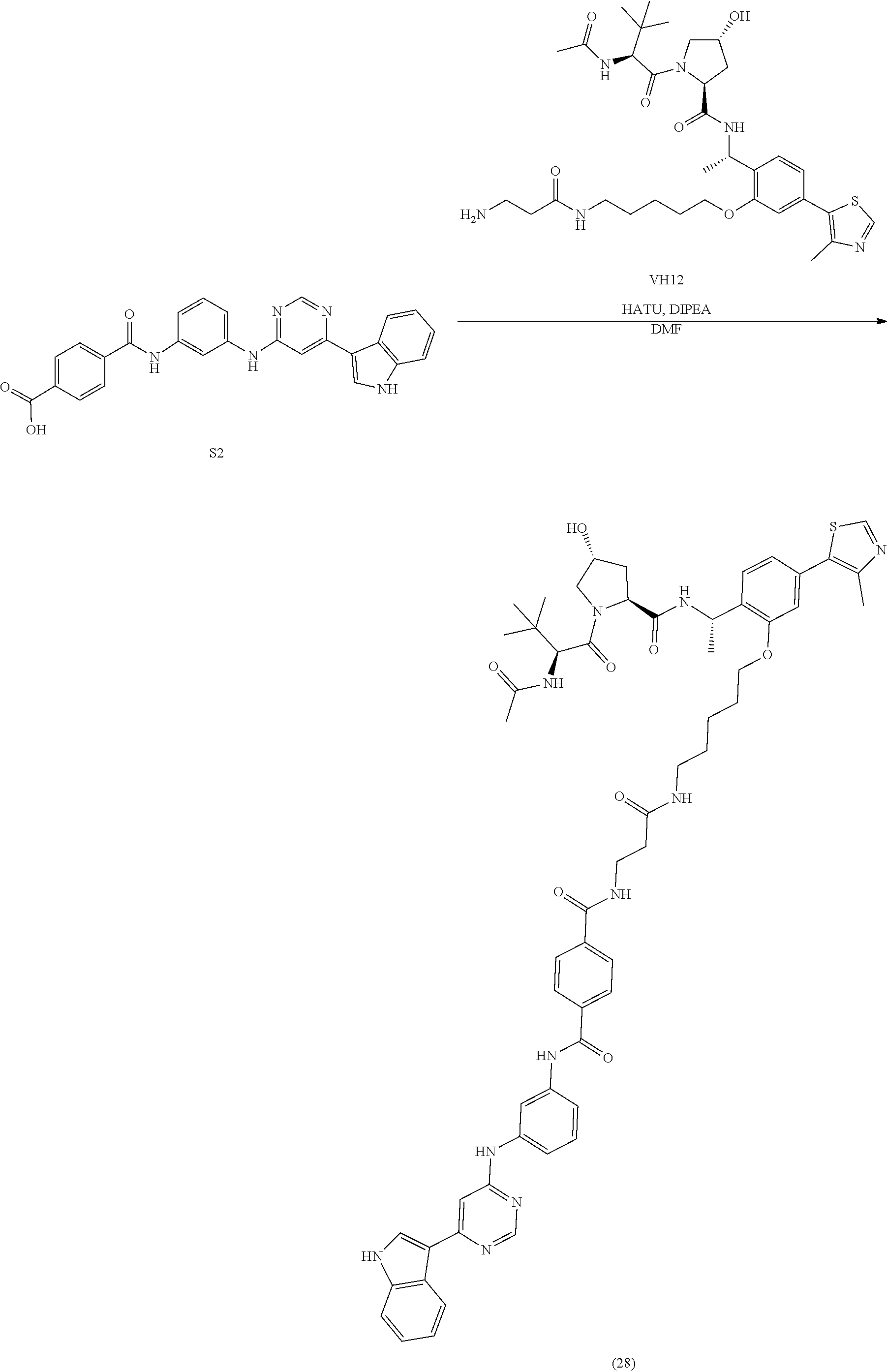

To a mixture of S2 (8.0 mg, 1.0 eq.), VL12 (14 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 28 (8.6 mg, 42%) as white solid.

LC-MS (ESI) m/z: 1090 [M+1]+.

¹H NMR (500 MHZ, DMSO-d₆) δ 12.34 (s, 1H), 10.94 (s, 1H), 10.52 (s, 1H), 8.97 (d, J=3.8 Hz, 1H), 8.88 (s, 1H), 8.70 (t, J=5.6 Hz, 1H), 8.45-8.35 (m, 2H), 8.30 (q, J=3.7, 2.9 Hz, 2H), 8.08-7.87 (m, 9H), 7.67-7.49 (m, 5H), 7.48-7.41 (m, 2H), 7.37-7.26 (m, 3H), 7.04-6.92 (m, 2H), 6.88 (dd, J=7.8, 1.7 Hz, 1H), 4.97 (p, J=7.0 Hz, 1H), 4.55-4.47 (m, 2H), 4.34 (t, J=3.7 Hz, 1H), 4.02 (t, J=6.4 Hz, 4H), 2.46 (s, 3H), 2.40 (t, J=7.2 Hz, 2H), 2.05-1.96 (m, 1H), 1.89 (d, J=3.2 Hz, 3H), 1.75 (q, J=6.9 Hz, 2H), 1.53-1.41 (m, 5H), 1.32 (d, J=6.8 Hz, 3H), 0.93 (d, J=3.3 Hz, 2H), 0.85 (s, 9H).

### Example 23: Synthesis of N1-(3-((6-(1H-indol-3-yl)pyrimidin-4-yl)amino)phenyl)-N4-(3-(((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)terephthalamide (29)

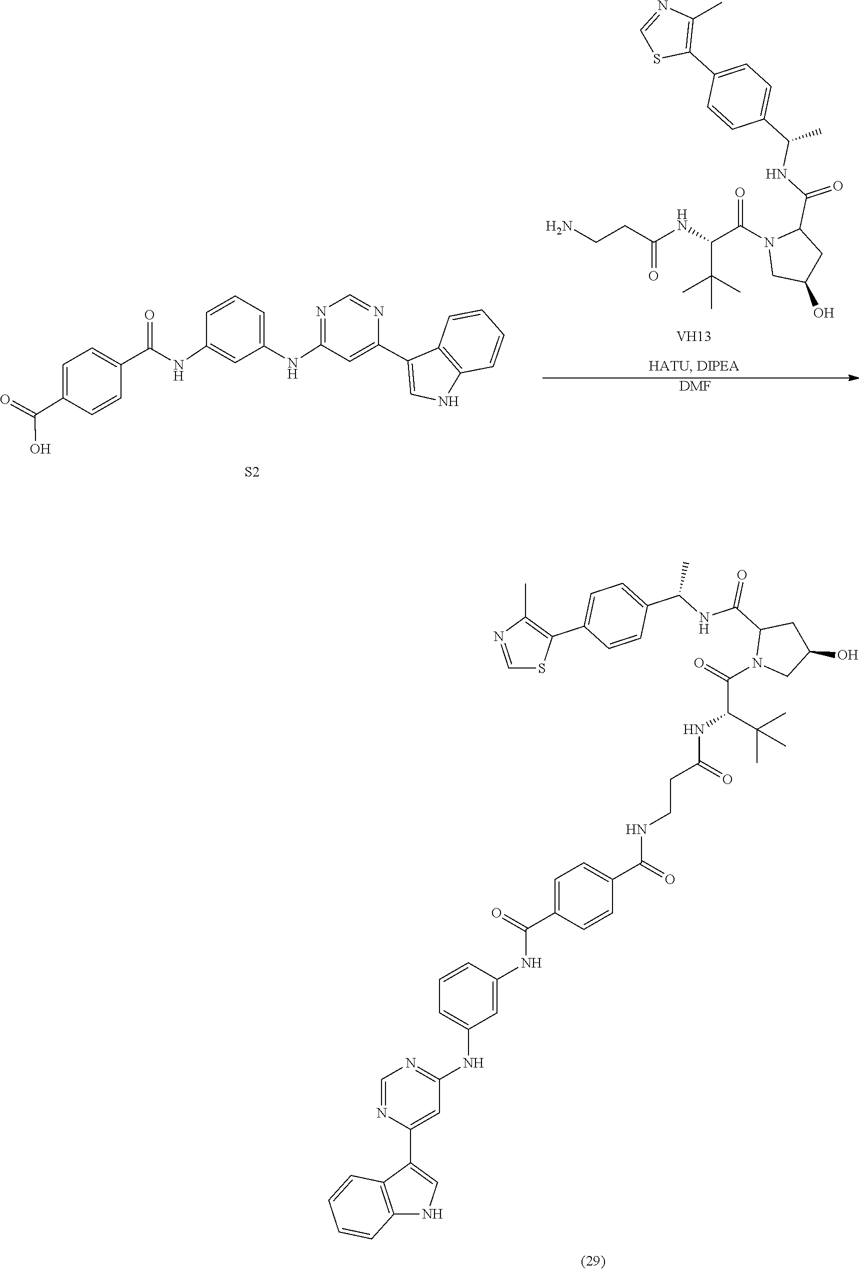

To a mixture of S2 (8.0 mg, 1.0 eq.), VL13 (10 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 29 (7.9 mg, 45%) as white solid.

LC-MS (ESI) m/z: 947[M+1]+.

¹H NMR (500 MHZ, DMSO-d₆) δ 12.30 (s, 1H), 10.86 (s, 1H), 10.51 (d, J=14.1 Hz, 1H), 8.99 (s, 1H), 8.87 (s, 1H), 8.63 (t, J=5.6 Hz, 1H), 8.41-8.34 (m, 2H), 8.32-8.26 (m, 1H), 8.09-7.94 (m, 5H), 7.63-7.59 (m, 1H), 7.56 (s, 1H), 7.52 (d, J=8.4 Hz, 1H), 7.47-7.41 (m, 3H), 7.41-7.36 (m, 2H), 7.35-7.27 (m, 2H), 4.91 (dt, J=15.6, 7.7 Hz, 1H), 4.55 (d, J=9.3 Hz, 1H), 4.44 (t, J=8.0 Hz, 1H), 4.30 (t, J=3.8 Hz, 1H), 2.59 (dt, J=14.8, 7.5 Hz, 1H), 2.46 (s, 3H), 2.42 (s, 1H), 2.03 (ddd, J=11.2, 7.7, 2.7 Hz, 1H), 1.81 (ddd, J=13.0, 8.5, 4.7 Hz, 1H), 1.38 (d, J=7.0 Hz, 3H), 0.93 (d, J=7.7 Hz, 9H).

### Example 24: Synthesis of N1-(3-((6-(1H-indol-3-yl)pyrimidin-4-yl)amino)phenyl)-N4-(2-((5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)amino)-2-oxoethyl)terephthalamide (30)

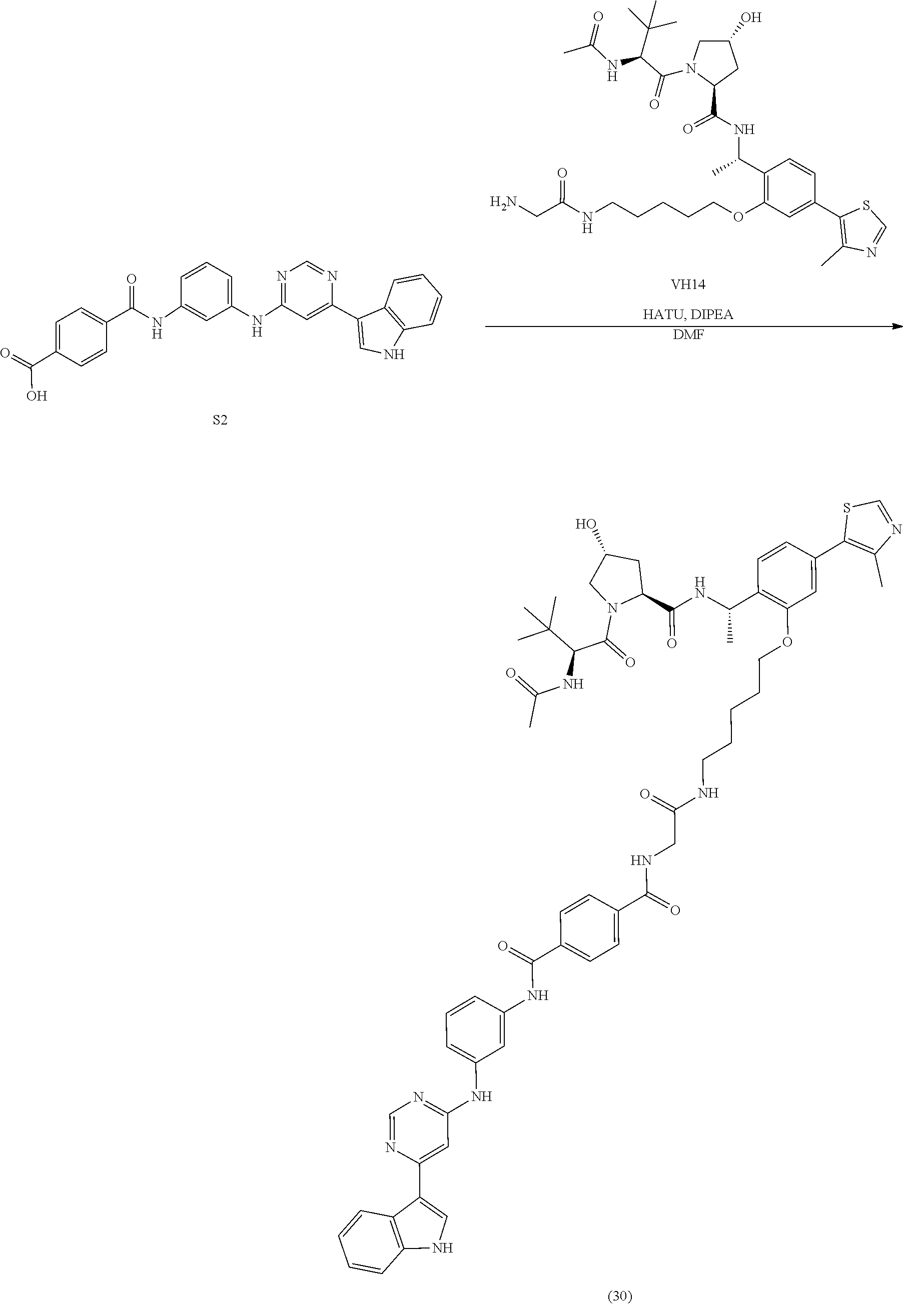

To a mixture of S2 (8.0 mg, 1.0 eq.), VL14 (14 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 30 (7.9 mg, 39%) as white solid.

LC-MS (ESI) m/z: 1076 [M+1]+.

¹H NMR (500 MHZ, DMSO-d₆) δ 12.28 (s, 1H), 10.82 (s, 1H), 10.53 (s, 1H), 8.98 (d, J=3.7 Hz, 1H), 8.87 (d, J=6.0 Hz, 2H), 8.42 (d, J=7.3 Hz, 1H), 8.36 (d, J=3.2 Hz, 1H), 8.30 (d, J=8.6 Hz, 1H), 8.06 (q, J=8.6 Hz, 5H), 8.00-7.92 (m, 2H), 7.64-7.54 (m, 3H), 7.52 (d, J=8.1 Hz, 1H), 7.47-7.41 (m, 2H), 7.35-7.26 (m, 2H), 7.04-6.94 (m, 1H), 6.89 (dd, J=7.8, 1.6 Hz, 1H), 5.21-5.15 (m, 1H), 4.98 (p, J=6.9 Hz, 1H), 4.55-4.46 (m, 3H), 4.35 (s, 2H), 4.06 (q, J=6.8, 5.1 Hz, 2H), 3.89 (d, J=5.8 Hz, 2H), 3.61 (t, J=6.8 Hz, 2H), 3.14 (d, J=6.3 Hz, 2H), 2.46 (s, 3H), 1.89 (d, J=3.5 Hz, 3H), 1.79 (t, J=6.3 Hz, 2H), 1.56-1.44 (m, 4H), 1.33 (t, J=6.3 Hz, 2H), 0.93 (d, J=3.3 Hz, 2H), 0.85 (s, 9H).

### Example 25: Synthesis of N1-(3-((6-(1H-indol-3-yl)pyrimidin-4-yl)amino)phenyl)-N4-(2-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)amino)-2-oxoethyl)terephthalamide (31)

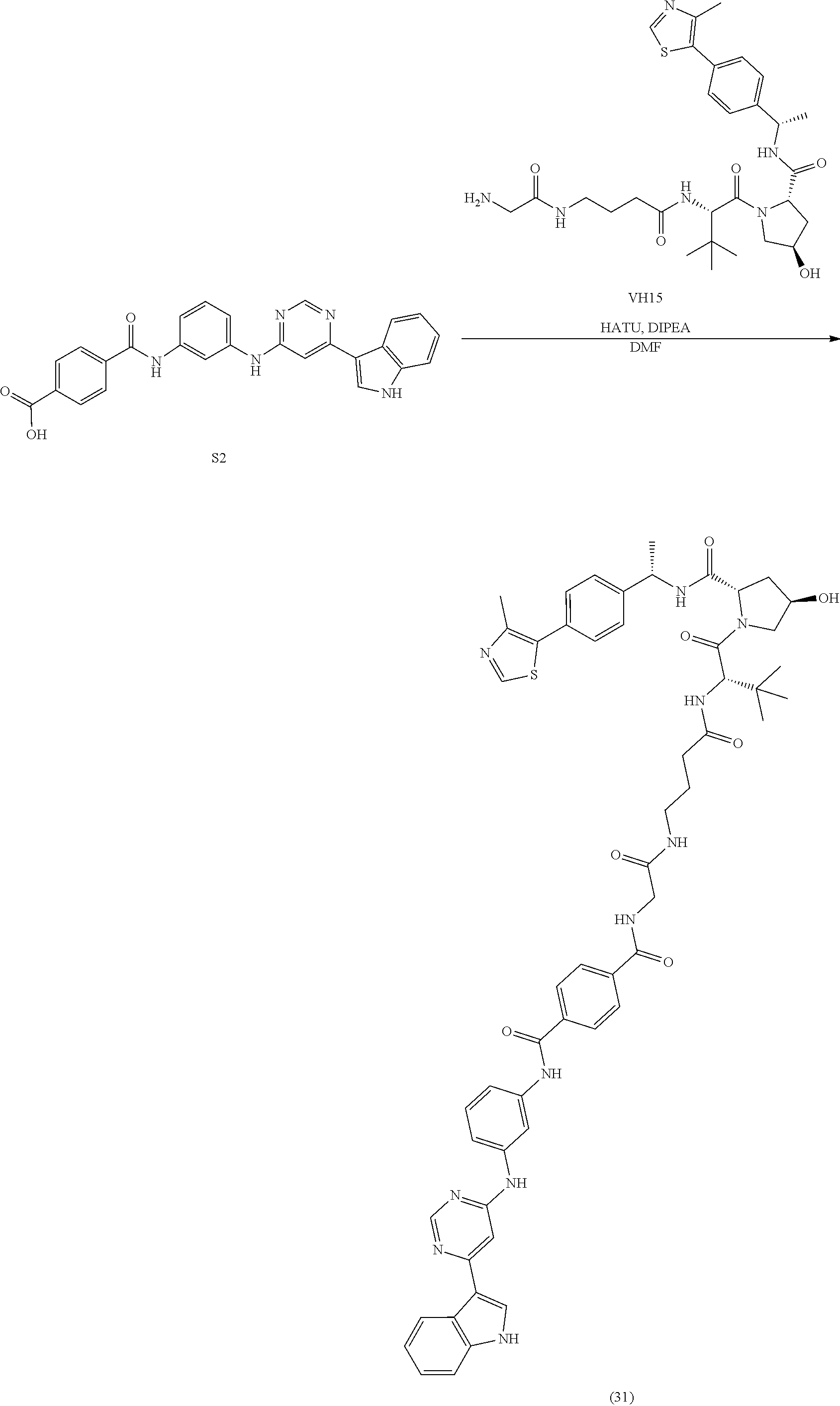

To a mixture of S2 (8.0 mg, 1.0 eq.), VL15 (12 mg, 1.0 eq.), and DIPEA (5.0 eq.) in DMF (0.5 mL) was added HATU (8.0 mg, 1.0 eq.). The resultant solution was stirred at room temperature overnight. The crude reaction mixture was purified by reverse-phase HPLC to yield bifunctional compound 31 (10.8 mg, 56%) as white solid.

LC-MS (ESI) m/z: 1019 [M+1]+.

¹H NMR (500 MHZ, DMSO-d₆) δ 12.30 (s, 1H), 10.84 (s, 1H), 10.54 (s, 1H), 8.99 (s, 1H), 8.91 (t, J=6.0 Hz, 1H), 8.87 (s, 1H), 8.38 (dd, J=7.9, 5.4 Hz, 2H), 8.31 (s, 1H), 8.11-7.98 (m, 6H), 7.85 (d, J=9.3 Hz, 1H), 7.63-7.49 (m, 3H), 7.44 (d, J=8.1 Hz, 4H), 7.40-7.36 (m, 2H), 7.32 (q, J=7.5 Hz, 2H), 4.92 (q, J=7.3 Hz, 1H), 4.54-4.39 (m, 2H), 4.29 (s, 1H), 3.89 (d, J=6.0 Hz, 2H), 3.65-3.55 (m, 2H), 3.09 (d, J=6.5 Hz, 2H), 2.46 (s, 3H), 2.28 (dt, J=14.8, 7.5 Hz, 1H), 2.17 (dt, J=14.5, 7.4 Hz, 1H), 2.02 (t, J=10.4 Hz, 1H), 1.80 (ddd, J=12.7, 8.5, 4.6 Hz, 1H), 1.65 (td, J=7.3, 3.1 Hz, 2H), 1.38 (d, J=7.0 Hz, 3H), 0.94 (d, J=5.3 Hz, 9H).

### Example 26: Synthesis s of N1-(4-((3-((6-(1H-indol-3-yl)pyrimidin-4-yl)amino)phenyl)carbamoyl)phenyl)-N3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)malonamide (32)

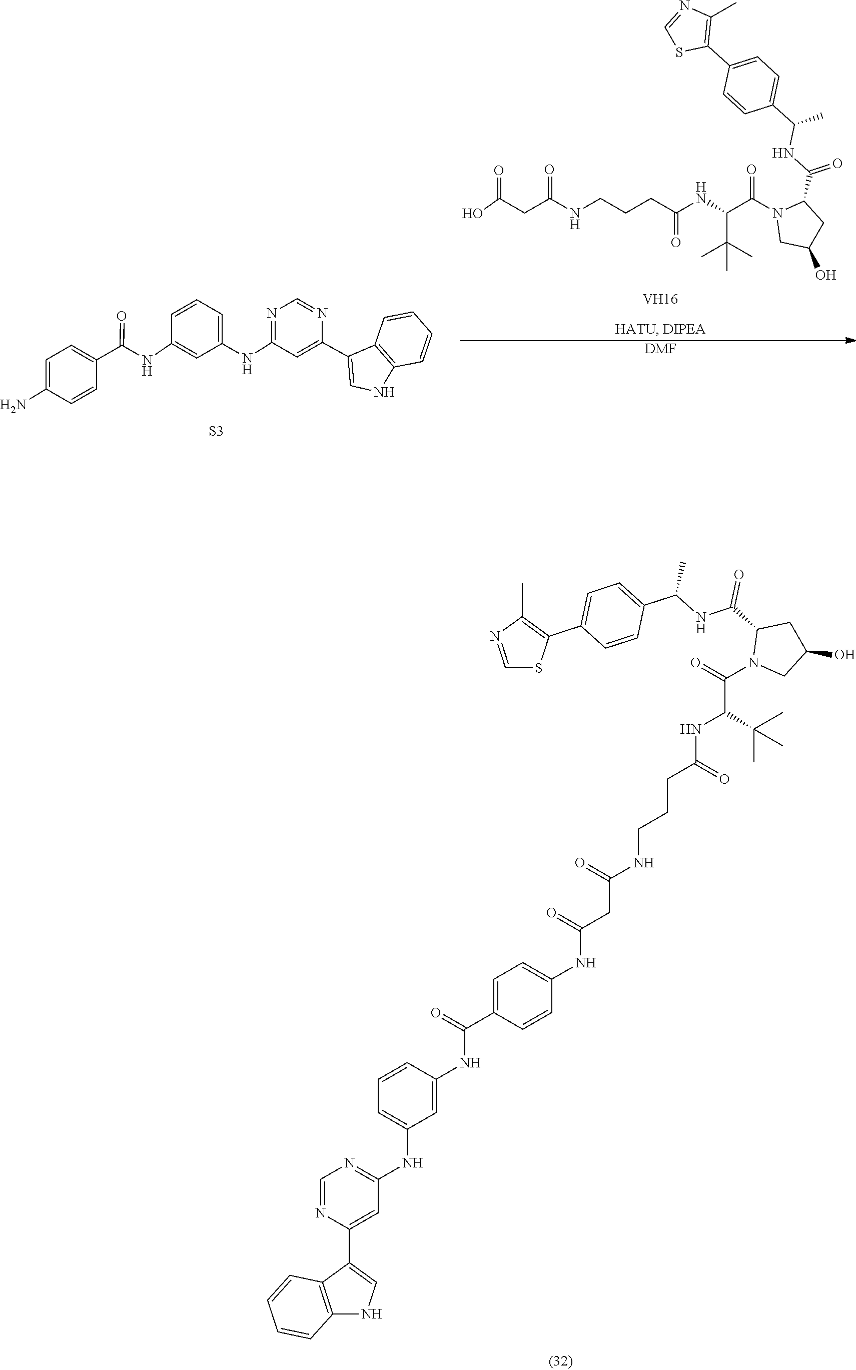

To a mixture of S3 (8.0 mg, 1.0 eq.), VL16 (15 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 32 (6.3 mg, 33%) as white solid.

LC-MS (ESI) m/z: 1018 [M+1]+.

¹H NMR (500 MHZ, DMSO-d₆) δ 12.28 (s, 1H), 10.81 (s, 1H), 10.43 (s, 1H), 10.31 (s, 1H), 8.99 (d, J=2.6 Hz, 1H), 8.86 (s, 1H), 8.37 (dd, J=13.5, 5.5 Hz, 2H), 8.29 (s, 1H), 8.13 (t, J=5.7 Hz, 1H), 8.04 (d, J=7.7 Hz, 1H), 7.98 (d, J=8.5 Hz, 2H), 7.85 (d, J=9.2 Hz, 1H), 7.76 (d, J=8.5 Hz, 2H), 7.61 (d, J=7.9 Hz, 1H), 7.56-7.47 (m, 2H), 7.46-7.36 (m, 5H), 7.31 (p, J=7.3 Hz, 2H), 4.92 (p, J=6.9 Hz, 1H), 4.48 (dt, J=40.9, 8.6 Hz, 2H), 4.29 (t, J=3.9 Hz, 1H), 3.09 (q, J=7.0 Hz, 2H), 2.46 (s, 3H), 2.36-2.10 (m, 4H), 2.08-1.97 (m, 1H), 1.80 (td, J=9.9, 9.1, 4.4 Hz, 2H), 1.65 (dp, J=16.1, 8.8, 7.8 Hz, 2H), 1.38 (d, J=7.0 Hz, 3H), 0.95 (d, J=4.7 Hz, 9H).

### Example 27: Synthesis of N1-(4-((3-((6-(1H-indol-3-yl)pyrimidin-4-yl)amino)phenyl)carbamoyl)phenyl)-N3-(5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)malonamide (33)

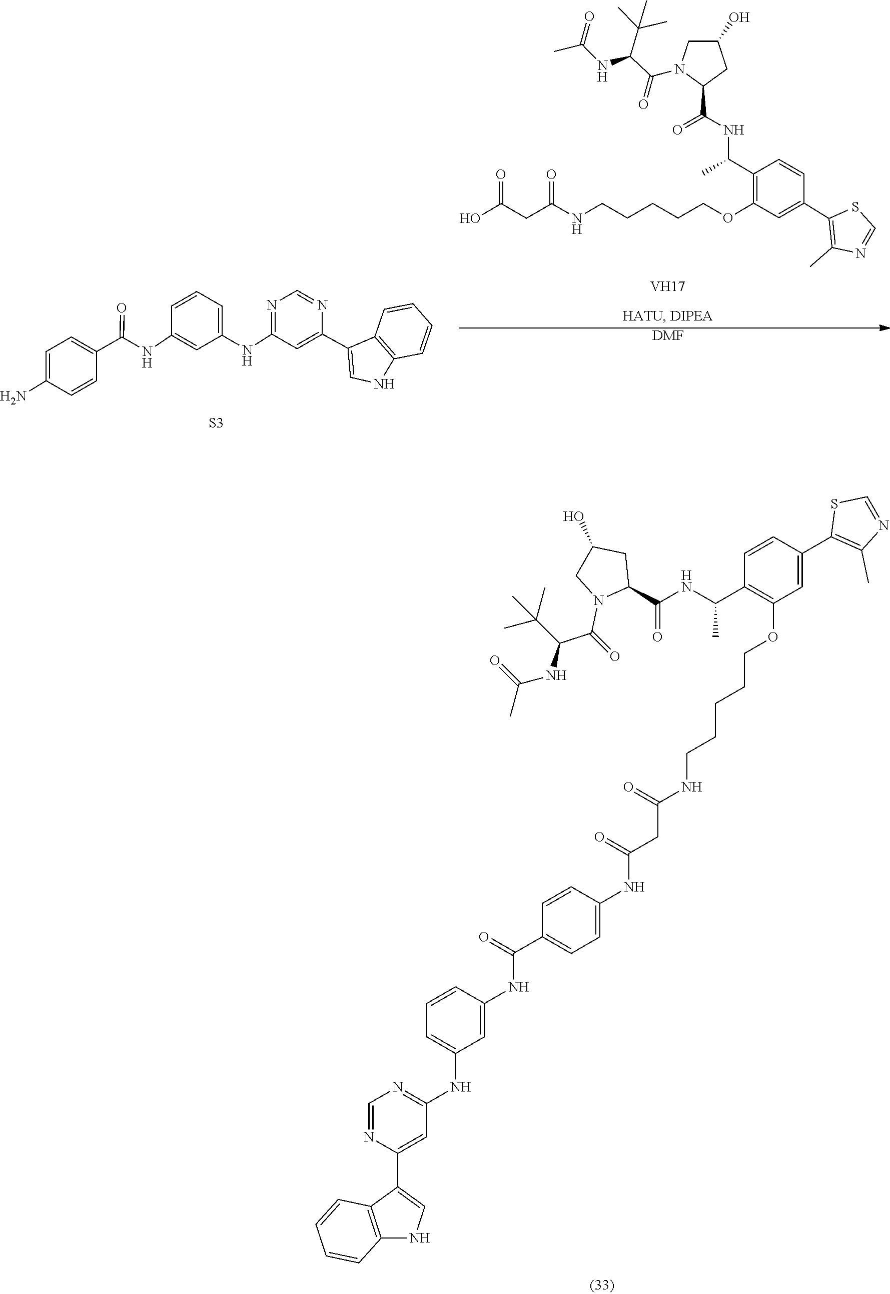

To a mixture of S3 (8.0 mg, 1.0 eq.), VL17 (17 mg, 1.0 eq.), and DIPEA (5.0 eq.) in DMF (0.5 mL) was added HATU (8.0 mg, 1.0 eq.). The resulting solution was stirred at room temperature overnight. The crude reaction mixture was purified by reverse-phase HPLC to yield bifunctional compound 33 (8.5 mg, 42%) as white solid.

LC-MS (ESI) m/z: 1076 [M+1]+.

¹H NMR (500 MHZ, DMSO-d₆) δ 12.29 (s, 1H), 10.82 (s, 1H), 10.41 (s, 1H), 10.30 (s, 1H), 8.98 (d, J=3.9 Hz, 1H), 8.87 (s, 1H), 8.42 (d, J=7.3 Hz, 1H), 8.36 (d, J=3.1 Hz, 1H), 8.28 (d, J=2.2 Hz, 1H), 8.12 (t, J=5.6 Hz, 1H), 8.04 (d, J=7.6 Hz, 1H), 7.96 (dd, J=12.7, 9.1 Hz, 3H), 7.78-7.69 (m, 2H), 7.61 (dd, J=8.3, 2.4 Hz, 2H), 7.57-7.47 (m, 2H), 7.46-7.39 (m, 2H), 7.31 (t, J=7.9 Hz, 2H), 7.05-6.94 (m, 1H), 6.89 (dd, J=7.8, 1.7 Hz, 1H), 4.98 (q, J=7.0 Hz, 1H), 4.56-4.48 (m, 2H), 4.32 (d, J=31.3 Hz, 2H), 4.05 (t, J=6.2 Hz, 2H), 3.62 (d, J=6.7 Hz, 2H), 3.14 (d, J=6.3 Hz, 2H), 2.46 (s, 3H), 2.07-1.97 (m, 1H), 1.89 (d, J=3.4 Hz, 3H), 1.79 (t, J=7.4 Hz, 3H), 1.51 (s, 4H), 1.33 (t, J=6.3 Hz, 3H), 0.94 (d, J=3.7 Hz, 2H), 0.86 (s, 9H).

### Example 28: Selective PIP4K2C Degradation with Inventive Bifunctional Compounds

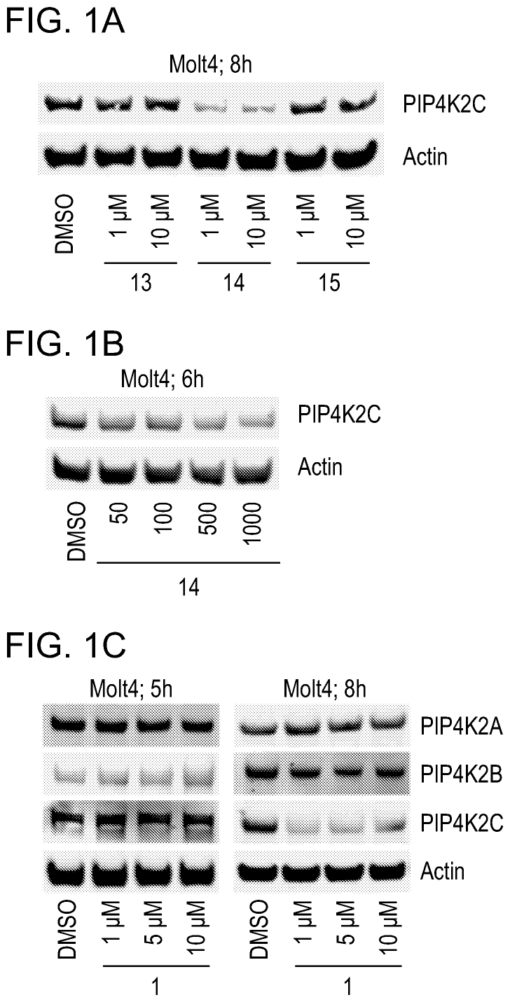

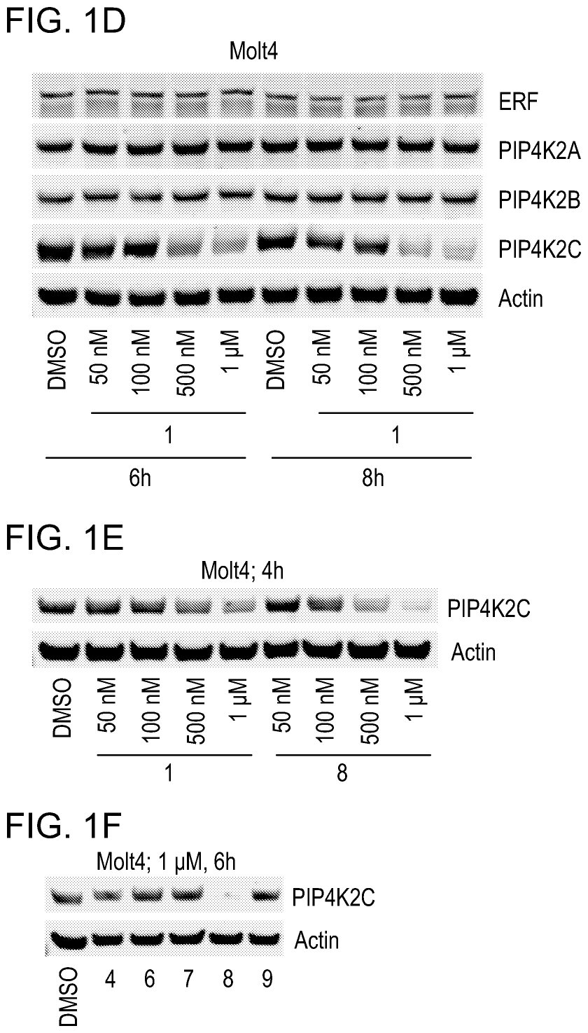

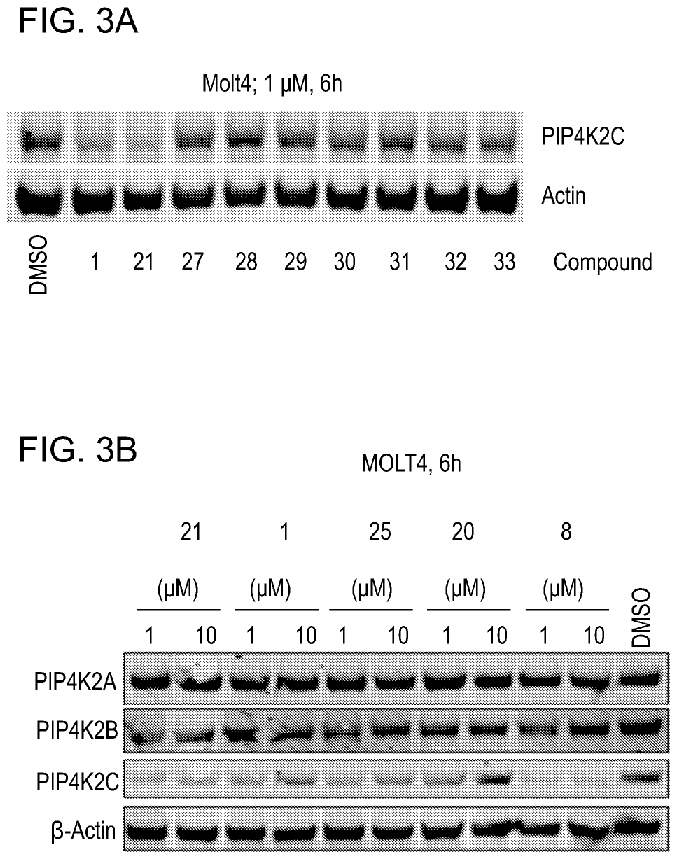

Molt4 cells were treated with the indicated compounds for the indicated time in FIG. 1A-FIG. 1F and FIG. 3A-FIG. 3B. Cells were lysed in M-PER™ buffer (Thermo Scientific) containing protease/phosphatase inhibitor cocktail (Roche®). Protein concentration was measured using a bicinchoninic acid (BCA) assay (Pierce™). Equivalent amounts of each sample were loaded on 4-12% Bis-Tris gels (Invitrogen™), transferred to nitrocellulose membranes, and immunoblotted with antibodies against the indicated proteins. IRDye®800-labeled goat anti-rabbit IgG and IRDye®680-labeled goat anti-mouse IgG (LI-COR®) secondary antibodies were purchased for LI-COR®, and membranes were detected on an Odyssey® detection system (LI-COR® Biosciences).

The results are summarized in FIG. 1A-FIG. 1F and FIG. 3A-FIG. 3B.

The results illustrated in FIG. 1A-FIG. 1F and FIG. 3A-FIG. 3B demonstrate that the inventive bifunctional compounds induced potent degradation of PIP4K2C at submicromolar concentrations while sparing PIP4K2A and PIP4K2B.

### Example 29: Proteomic Analysis

Mass spectrometry profiling (e.g., thalidomide, lenalidomide, and pomalidomide) was performed as illustrated in Donovan et al., eLife 7:e38430 (2018) and Sievers et al., Science 362: eaat0572 (2018).

### Sample Preparation TMT LC-MS3 Mass Spectrometry

Molt4 cells were treated with DMSO or the indicated compound for 5 h, and cells were harvested by centrifugation. Lysis buffer (8 M urea, 50 mM NaCl, 50 mM 4-(2hydroxyethyl)-1-piperazineethanesulfonic acid (EPPS) pH 8.5, 1× Roche protease inhibitor, and 1× Roche® PhosStop™) was added to the cell pellets and cells were homogenized by 20 passes through a 21-gauge (1.25 in. long) needle to achieve a cell lysate with a protein concentration between 0.5 and 4 mg mL-1. The homogenized sample was clarified by centrifugation at 20,000×g for 10 min at 4° C. A micro-BCA assay (Pierce™) was used to determine the final protein concentration in the cell lysate. 200 μg protein for each sample were reduced and alkylated as previously described (An et al., Nat. Commun. 8:15398 (2017)). Proteins were precipitated using methanol/chloroform. In brief, four volumes of methanol were added to the cell lysate, followed by one volume of chloroform, and finally three volumes of water. The mixture was vortexed and centrifuged at 14,000×g for 5 min to separate the chloroform phase from the aqueous phase. The precipitated protein was washed with three volumes of methanol, centrifuged at 14,000×g for 5 min, and the resulting washed precipitated protein was allowed to air dry. Precipitated protein was resuspended in 4 M urea, 50 mM 4-(2-hydroxyethyl)-1-piperazineethanesulfonic acid (HEPES) pH 7.4, followed by dilution to 1 M urea with the addition of 200 mM EPPS pH 8 for digestion with LysC (1:50; enzyme:protein) for 12 h at room temperature. The LysC digestion was diluted to 0.5 M urea, 200 mM EPPS pH 8, and then digested with trypsin (1:50; enzyme:protein) for 6 h at 37° C. Tandem mass tag (TMT) reagents (Thermo Fisher Scientific) were dissolved in anhydrous acetonitrile (ACN) according to manufacturer's instructions. Anhydrous ACN was added to each peptide sample to a final concentration of 30% v/v, and labeling was induced with the addition of TMT reagent to each sample at a ratio of 1:4 peptide:TMT label. The 10-plex labeling reactions were performed for 1.5 h at room temperature and the reaction quenched by the addition of 0.3% hydroxylamine for 15 min at room temperature. The sample channels were combined in a 1:1:1:1:1:1:1:1:1:1 ratio, desalted using C18 solid phase extraction cartridges (Waters®) and analyzed by liquid chromatography-mass spectrometry (LC-MS) for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®, Waters®). Samples were then offline fractionated into 96 fractions by high pH reverse-phase high performance liquid chromatography (HPLC) (Agilent LC1260) through an aeris peptide xb-c18 column (Phenomenex®) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 or 48 fractions, and every fraction was used for subsequent mass spectrometry analysis.

Data were collected using an Orbitrap Fusion™ Lumos™ mass spectrometer (Thermo Fisher Scientific, San Jose, CA, USA) coupled with a Proxeon EASY-nLC™ 1200 LC pump (Thermo Fisher Scientific). Peptides were separated on a 50 cm and 75 μm inner diameter EASY-Spray™ column (ES803, Thermo Fisher Scientific). Peptides were separated using a 3 h gradient of 6-27% acetonitrile in 1.0% formic acid with a flow rate of 300 nL/min.

Each analysis used an MS3-based TMT method as described previously (McAlister et al., Anal. Chem. 86:7150-7158 (2014)). The data were acquired using a mass range of m z 350-1350, resolution 120,000, AGC target 1×10⁶, maximum injection time 100 ms, dynamic exclusion of 90 s for the peptide measurements in the Orbitrap. Data-dependent MS2 spectra were acquired in the ion trap with a normalized collision energy (NCE) set at 35%, AGC target set to 1.8×10⁴, and a maximum injection time of 120 ms. MS3 scans were acquired in the Orbitrap with a HCD collision energy set to 55%, AGC target set to 1.5×10⁵, maximum injection time of 150 ms, resolution at 50,000, and with a maximum synchronous precursor selection (SPS) precursors set to 10.

### LC-MS Data Analysis

Proteome Discoverer™ 2.2 (Thermo Fisher) was used for RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 20 ppm, fragment ion mass tolerance of 0.6 Da, static alkylation of cysteine (57.02146 Da), static TMT labeling of lysine residues and N-termini of peptides (229.16293 Da), and variable oxidation of methionine (15.99491 Da). TMT reporter ion intensities were measured using a 0.003 Da window around the theoretical m z for each reporter ion in the MS3 scan. Peptide spectral matches with poor-quality MS3 spectra were excluded from quantitation (summed signal-to-noise across 10 channels >200 and precursor isolation specificity <0.5). Reporter ion intensities were normalized and scaled using in-house scripts and the R framework (R Core Team, R Foundation for Statistical Computing, Vienna, Austria (2013)). Statistical analysis was carried out using the limma package within the R framework (Ritchie et al., Nucleic Acids Res. 43:e47 (2015)).

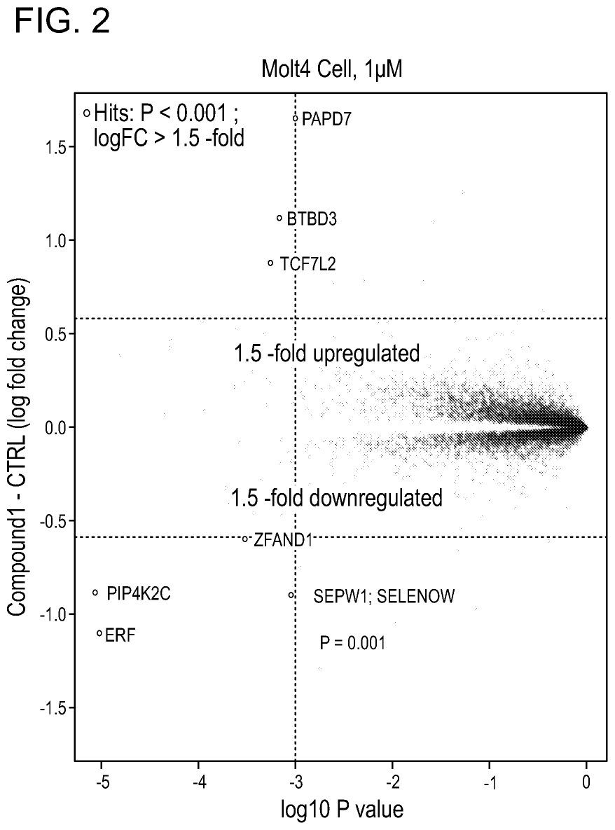

The results are summarized in FIG. 2.

As illustrated in FIG. 2, proteomics analysis of Molt4 cells treated with Compound 1 at 1 μM for 5 h demonstrated selective degradation of PIP4K2C, with no detectable change in abundance of PIP4K2A and PIP4K2B. While ETS2 Repressor Factor (ERF) scored as a potential degraded protein, as seen in FIG. 1D, ERF was not validated as a confirmed degraded protein by proteomics analysis.

TABLE 1
IC₅₀ values for bifunctional compound 1.

IC₅₀ (μM)

| Compound | PIP4K2A | PIP4K2B |
|---|---|---|
| 1 | >10 | 14 |

The data in table 1 show that bifunctional compound 1 has minimal biochemical binding affinity for PIP4K2A and PIP4K2B.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bifunctional compound, comprising a targeting ligand that binds at least one of phosphatidylinositol-5-phosphate 4-kinase type 2 alpha (PIP4K2A), PIP4K2B, and PIP4K2C and a degron covalently attached to the targeting ligand by a linker, wherein the compound has a structure represented by formula (I): 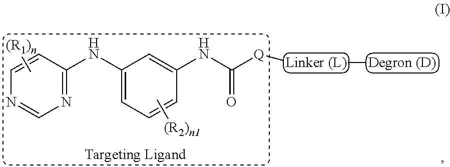
or a pharmaceutically acceptable salt or stereoisomer thereof, wherein:
Q represents a bond or 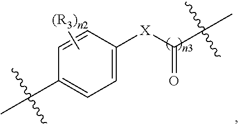
wherein X is a bond, CH₂, NH, or O;
each R₁ independently represents optionally substituted aryl, optionally substituted heteroaryl having 1-3 heteroatoms selected from N, O, and S, or NR₄R₅;
each R₂ and R₃ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
each R₄ and R₅ independently represents H, optionally substituted (C₁-C₆) alkyl, optionally substituted (C₁-C₆) haloalkyl, optionally substituted (C₁-C₆) alkoxy, optionally substituted (C₁-C₆) haloalkoxy, optionally substituted C₅-C₆ carbocyclyl or optionally substituted C₅-C₆ heterocarbocyclyl;
n represents 1, 2, or 3;
n₁ and n₂ independently represent 1, 2, 3, or 4;
n₃ is 0 or 1; and the
degron is a moiety that binds an E3 ubiquitin ligase.

2. The bifunctional compound of claim 1, wherein Q is a bond.

3. The bifunctional compound of claim 1, wherein Q is

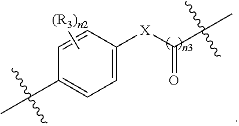

4. The bifunctional compound of claim 3, wherein X is NH.

5. The bifunctional compound of claim 1, wherein R₁ is optionally substituted aryl.

6. The bifunctional compound of claim 5, wherein the optionally substituted aryl is optionally substituted phenyl.

7. The bifunctional compound of claim 1, wherein R₁ is

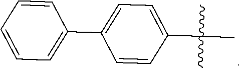 [SMILES: *c1ccc(-c2ccccc2)cc1]

8. The bifunctional compound of claim 1, wherein R₁ is optionally substituted heteroaryl having 1-3 heteroatoms selected from N, O, and S.

9. The bifunctional compound of claim 8, wherein the optionally substituted heteroaryl is optionally substituted indole, indazole, or azaindole.

10. The bifunctional compound of claim 9, wherein R₁ is

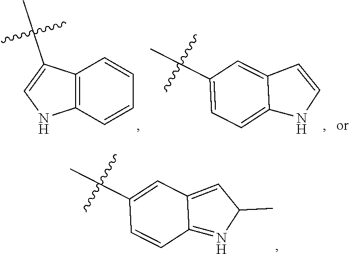 [SMILES: *c1c[nH]c2ccccc12 ; *c1ccc2[nH]ccc2c1 ; *c1ccc2c(c1)=CC(C)N=2]

11. The bifunctional compound of claim 1, wherein R₁ is NR₄R₅.

12. The bifunctional compound of claim 11, wherein one of R₄ and R₅ is H, optionally substituted C₅-C₆ carbocyclyl or optionally substituted C₅-C₆ heterocarbocyclyl.

13. (canceled)

14. The bifunctional compound of claim 12, wherein one of R₄ and R₅ is optionally substituted aryl.

15. The bifunctional compound of claim 14, wherein the optionally substituted aryl is optionally substituted phenyl.

16. The bifunctional compound of claim 11, wherein R₁ is

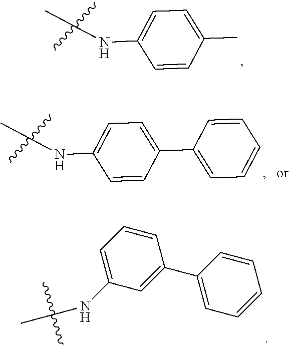

17. The bifunctional compound of claim 1, wherein n is 1.

18. The bifunctional compound of claim 1, wherein R₂ and R₃ are H.

19. The bifunctional compound of claim 1, wherein the targeting ligand is represented by any one of structures TL1-TL8:

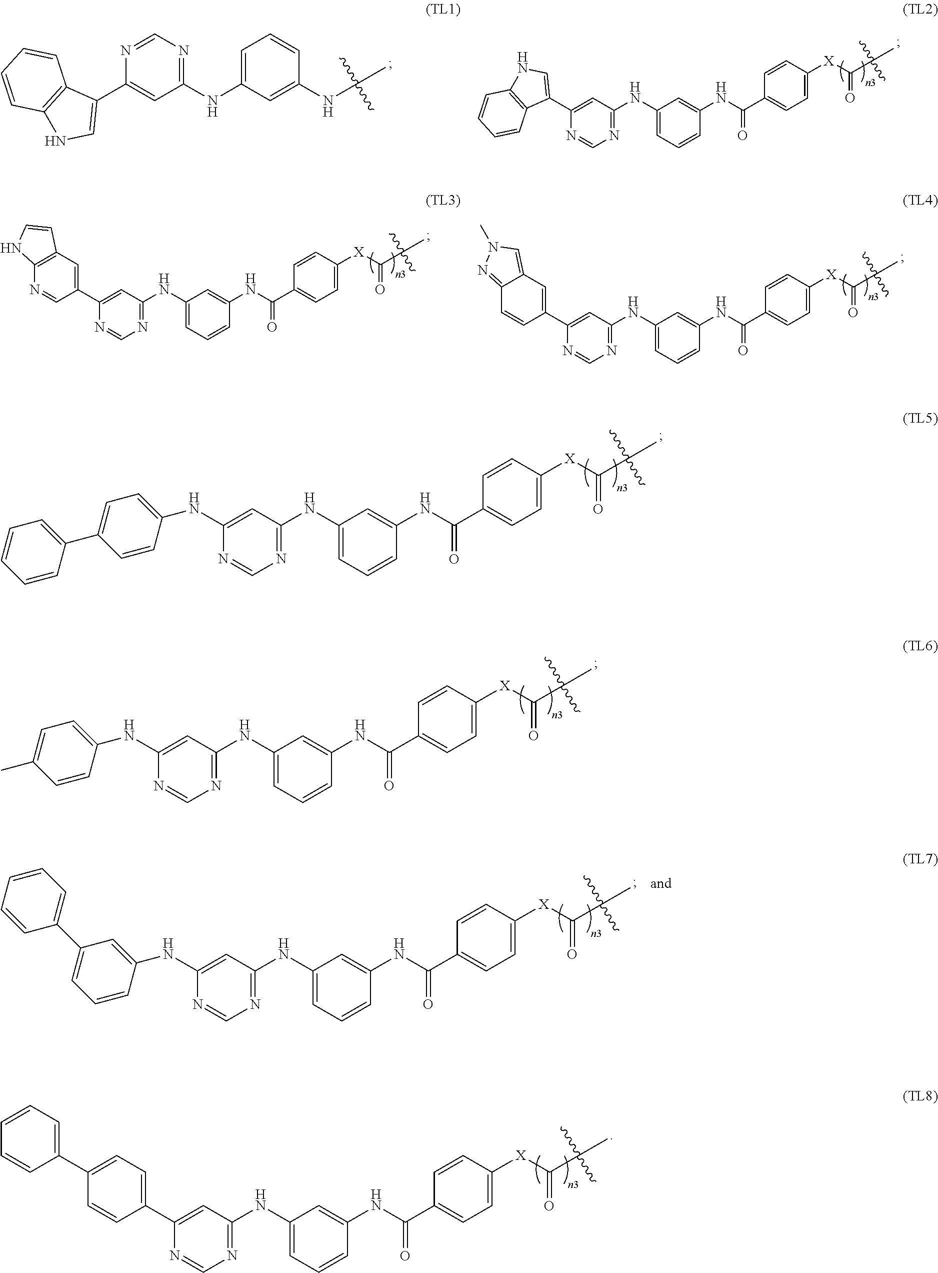

20. (canceled)

21. The bifunctional compound of claim 1, wherein the linker comprises an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

22. The bifunctional compound of claim 21, wherein the alkylene chain has 3-12 alkylene units.

23. The bifunctional compound of claim 1, wherein the linker comprises a polyethylene glycol chain which may be interrupted by, and/or terminate (at either or both termini) at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′), —C₃₋₁₂ N(R′)S(O)—, —S(O)N(R′)—, —N(R′)S(O)₂N(R′)—, —N(R′)S(O)N(R′)—, carbocyclene, 3- to 12-membered heterocyclene, 5- to 12-membered heteroarylene or any combination thereof, wherein R′ is H or C₁-C₆ alkyl, wherein the one or both terminating groups may be the same or different.

24. The bifunctional compound of claim 23, wherein the polyethylene glycol chain has 1-6 PEG units.

25. The bifunctional compound of claim 1, wherein the linker is represented by any one of structures L10 to L33:

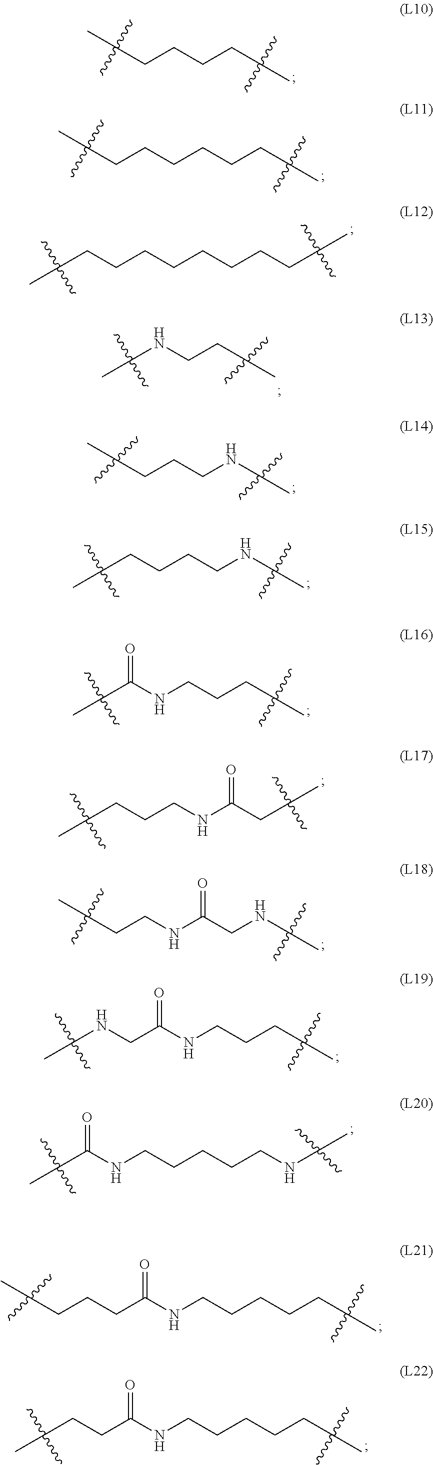

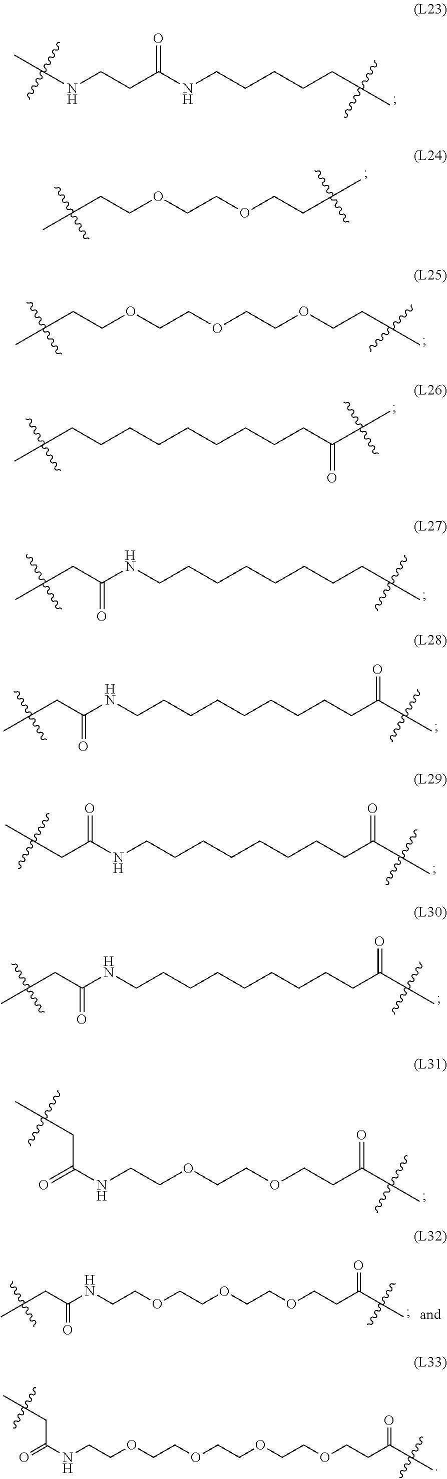

26. The bifunctional compound of claim 1, wherein the degron binds von Hippel Landau tumor suppressor (VHL) and the degron is represented by any one of structures (D1-a) to (D1-f);

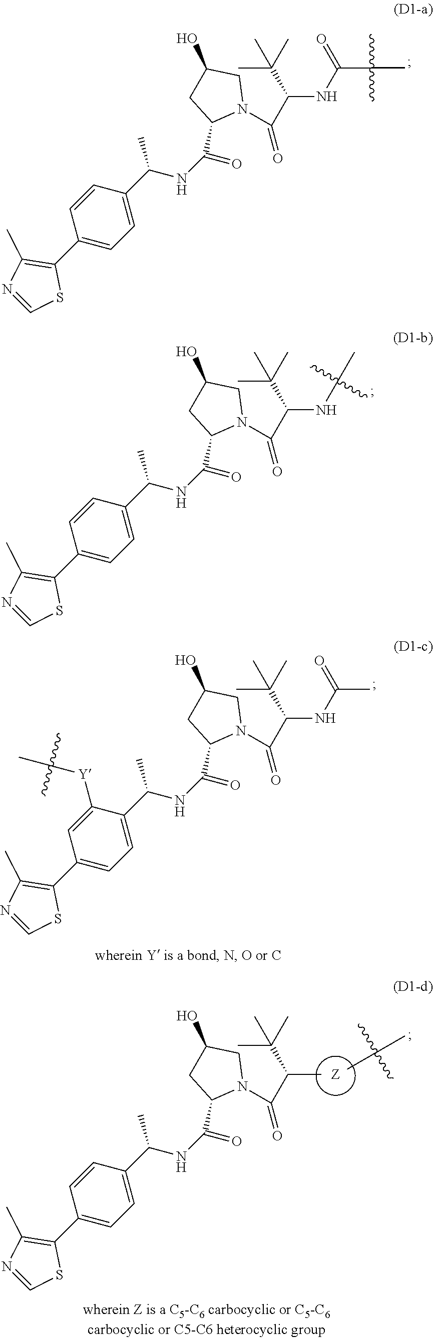

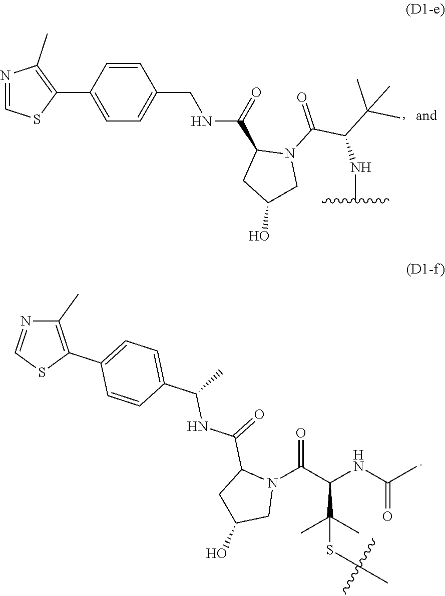 [SMILES: *N[C@H](C(=O)N1C[C@H](O)C[C@H]1C(=O)NCc1ccc(-c2scnc2C)cc1)C(C)(C)C ; *SC(C)(C)[C@H](NC(C)=O)C(=O)N1C[C@H](O)CC1C(=O)N[C@@H](C)c1ccc(-c2scnc2C)cc1]

27. (canceled)

28. The bifunctional compound of claim 26, wherein Z is

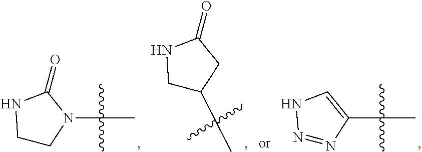 [SMILES: *N1CCNC1=O ; *C1CNC(=O)C1 ; *c1c[nH]nn1]

29. The bifunctional compound of claim 1, wherein the degron binds cereblon (CRBN) and the degron is represented by any one of structures (D2-a) to (D2-d): 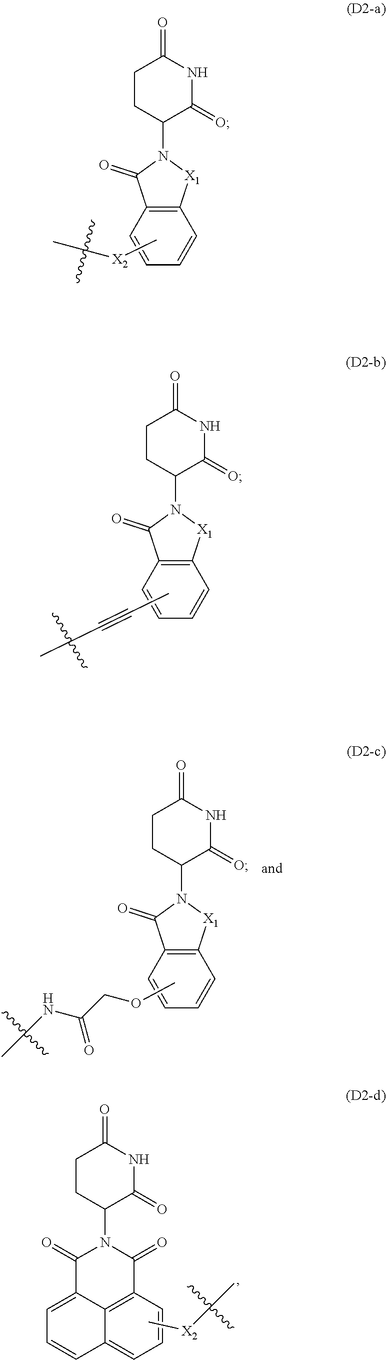
wherein X₁ is CH₂ or C(O) and X₂ is CR″₁R″₂, NR″₁R″₂, NH, O, or S, wherein R″₁ and R″₂ are independently H, halogen, OH, NH₂, C₁-C₃ alkyl, C₁-C₃ alkoxyl, or C₁-C₃ alkylamine, or R″₁ and R″₂, together with the atoms to which they are bound, form a C₃-C₇ carbocyclic or C₃-C₇ heterocyclic ring.

30. (canceled)

31. The bifunctional compound of claim 29, wherein R″₁ and R″₂, together with the atoms to which they are bound, form an azetidine, piperidine, pyrrolidine, cyclobutane, or cyclohexane ring.

32. The bifunctional compound of claim 1, which represented by any one of structures 1-26: 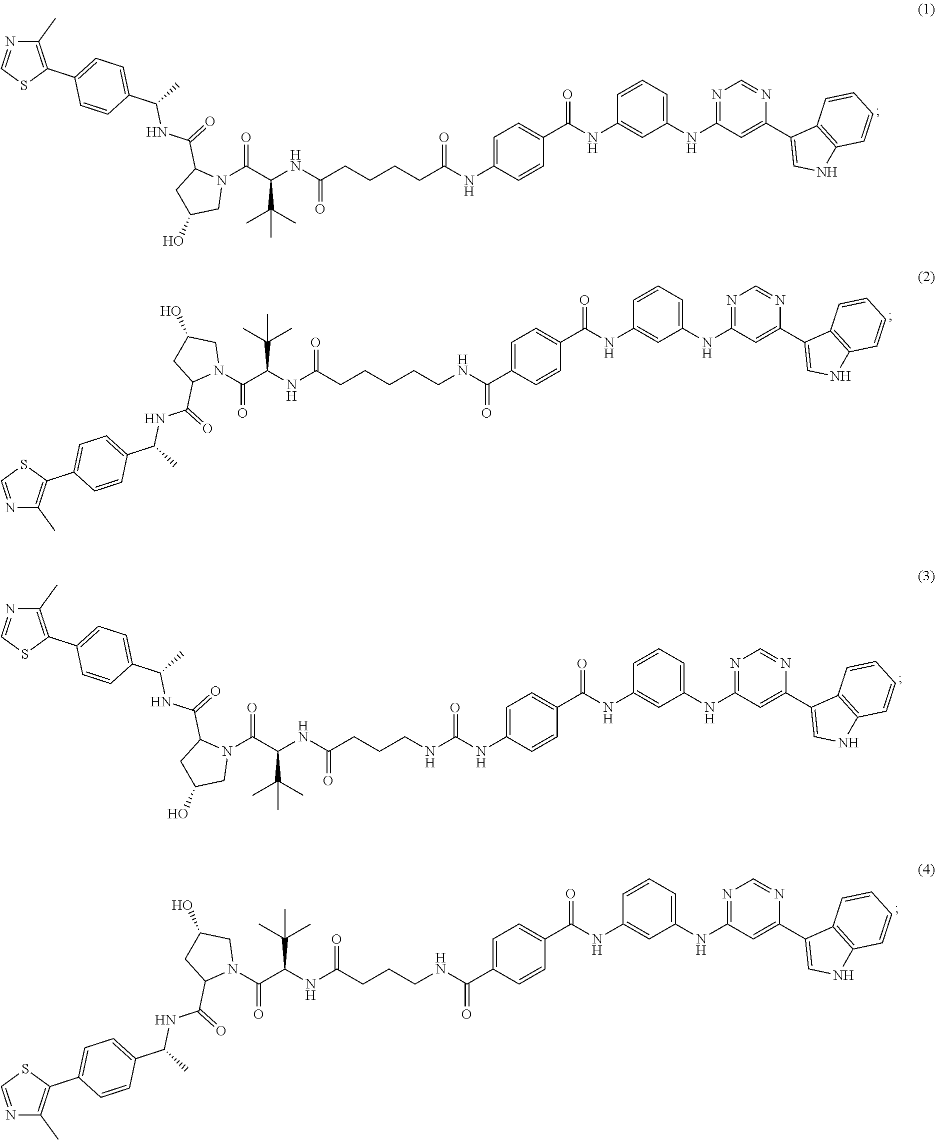 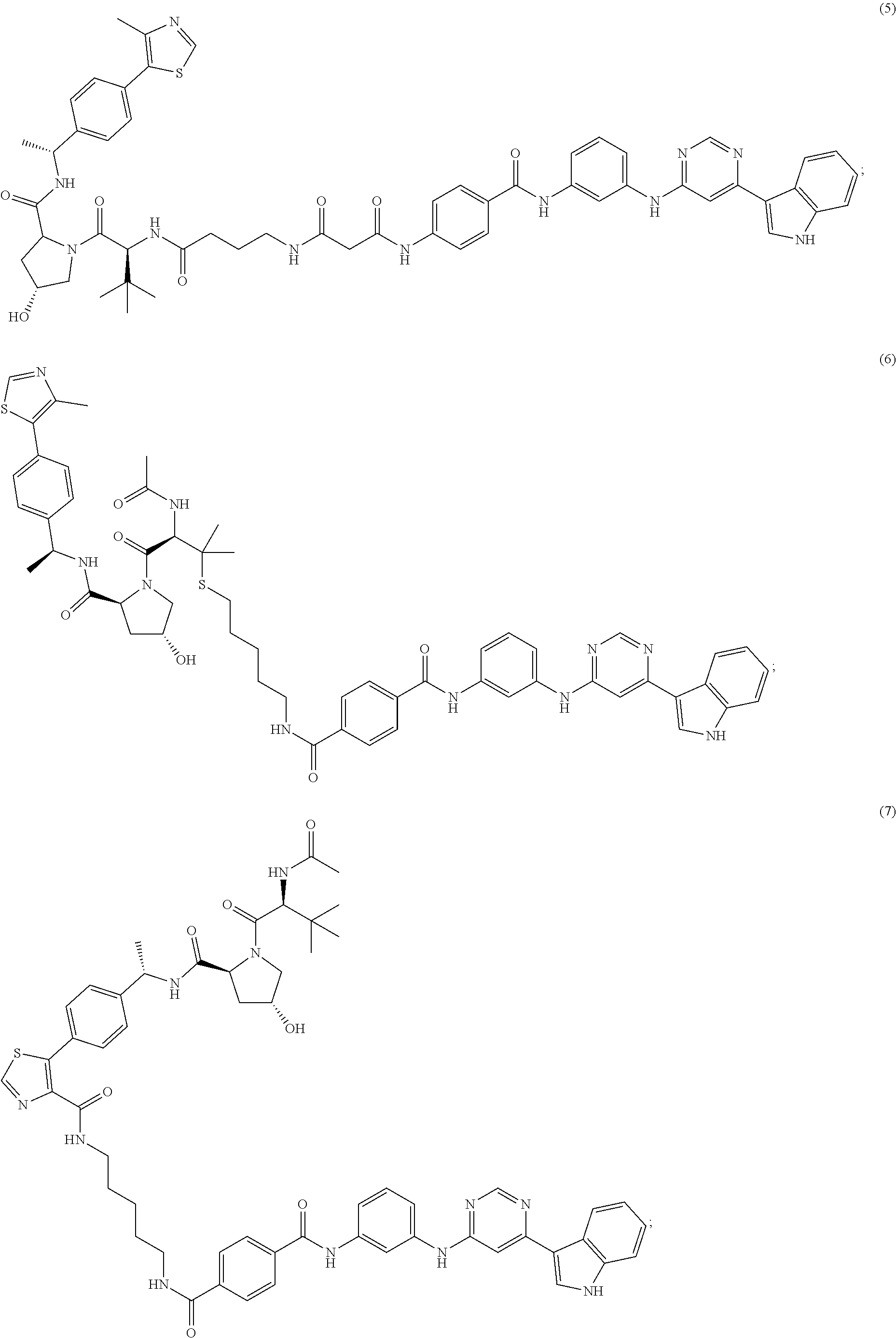 [SMILES: Cc1ncsc1-c1ccc([C@@H](C)NC(=O)C2C[C@@H](O)CN2C(=O)[C@@H](NC(=O)CCCNC(=O)CC(=O)Nc2ccc(C(=O)Nc3cccc(Nc4cc(-c5c[nH]c6ccccc56)ncn4)c3)cc2)C(C)(C)C)cc1 ; CC(=O)N[C@H](C(=O)N1C[C@H](O)C[C@H]1C(=O)N[C@@H](C)c1ccc(-c2scnc2C)cc1)C(C)(C)SCCCCCNC(=O)c1ccc(C(=O)Nc2cccc(Nc3cc(-c4c[nH]c5ccccc45)ncn3)c2)cc1 ; CC(=O)N[C@H](C(=O)N1C[C@H](O)C[C@H]1C(=O)N[C@@H](C)c1ccc(-c2scnc2C(=O)NCCCCCNC(=O)c2ccc(C(=O)Nc3cccc(Nc4cc(-c5c[nH]c6ccccc56)ncn4)c3)cc2)cc1)C(C)(C)C] 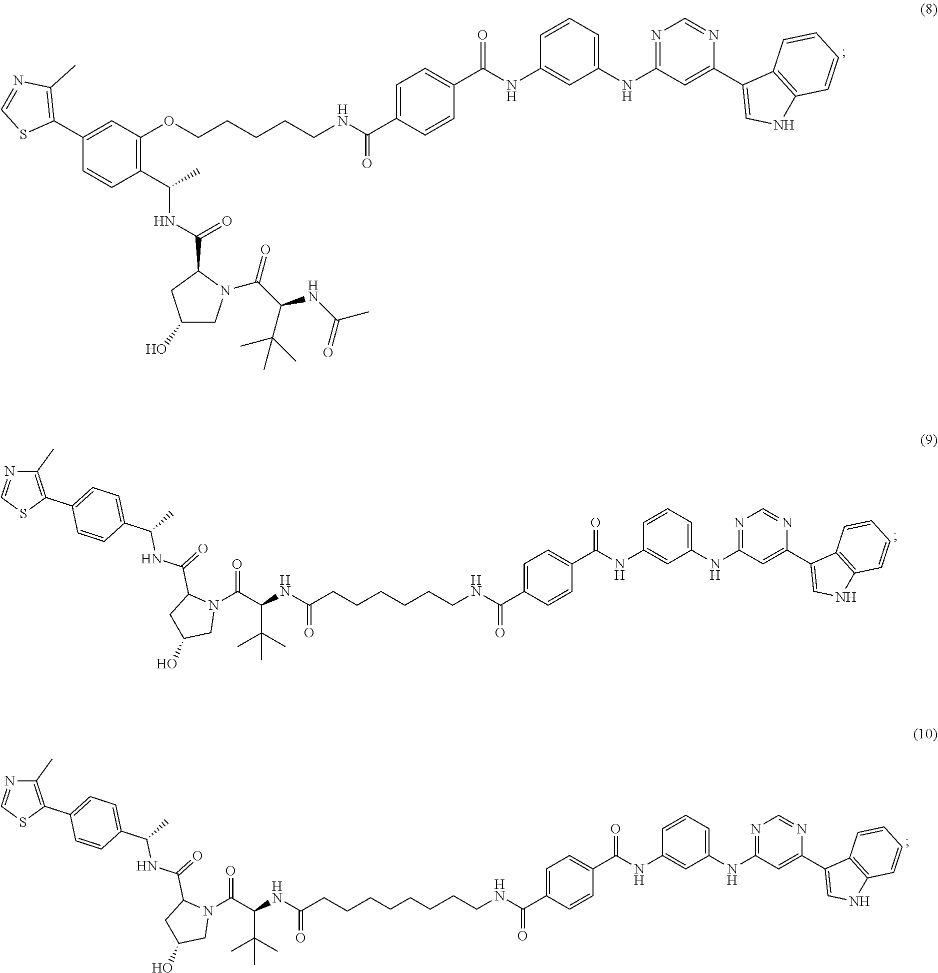 [SMILES: CC(=O)N[C@H](C(=O)N1C[C@H](O)C[C@H]1C(=O)N[C@@H](C)c1ccc(-c2scnc2C)cc1OCCCCCNC(=O)c1ccc(C(=O)Nc2cccc(Nc3cc(-c4c[nH]c5ccccc45)ncn3)c2)cc1)C(C)(C)C ; Cc1ncsc1-c1ccc([C@H](C)NC(=O)C2C[C@@H](O)CN2C(=O)[C@@H](NC(=O)CCCCCCNC(=O)c2ccc(C(=O)Nc3cccc(Nc4cc(-c5c[nH]c6ccccc56)ncn4)c3)cc2)C(C)(C)C)cc1 ; Cc1ncsc1-c1ccc([C@H](C)NC(=O)C2C[C@@H](O)CN2C(=O)[C@@H](NC(=O)CCCCCCCCNC(=O)c2ccc(C(=O)Nc3cccc(Nc4cc(-c5c[nH]c6ccccc56)ncn4)c3)cc2)C(C)(C)C)cc1] 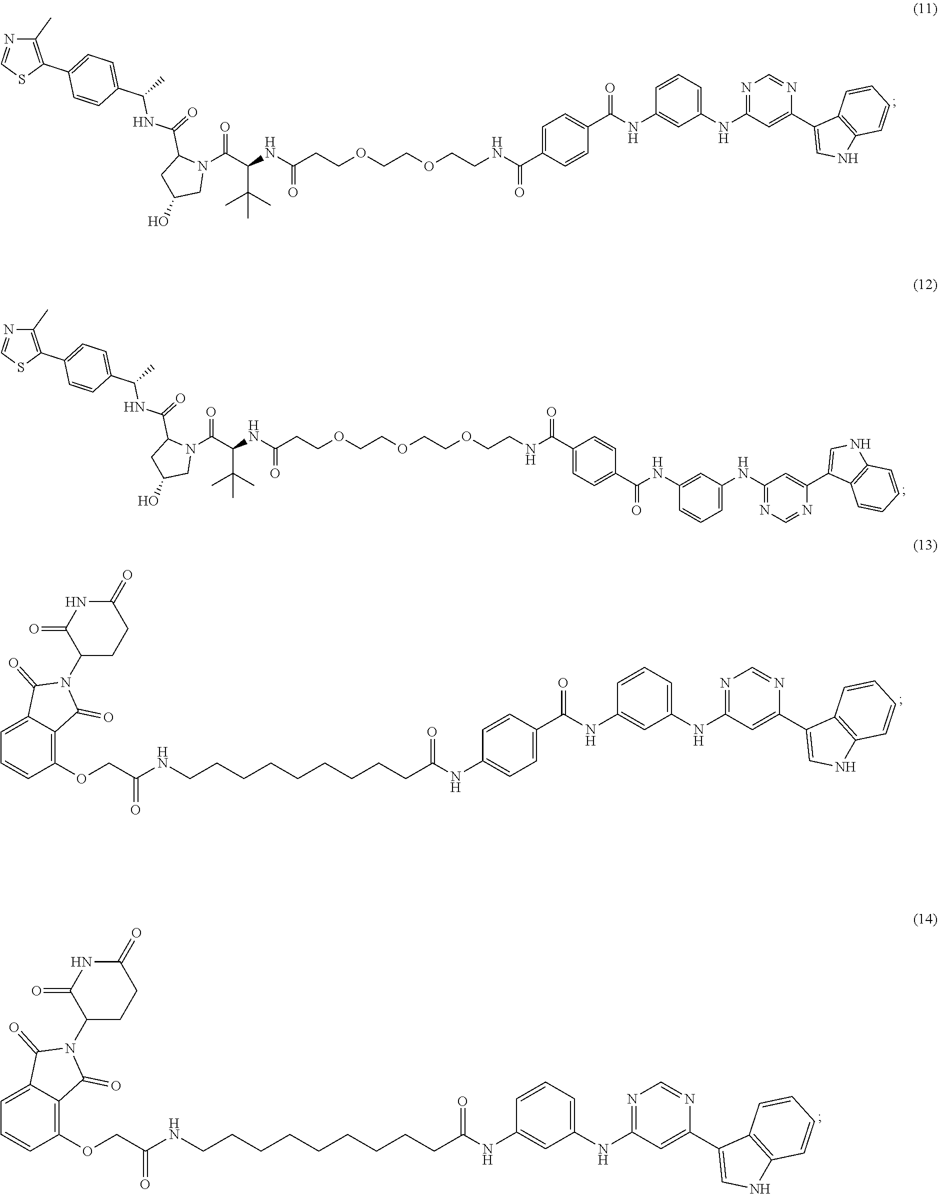 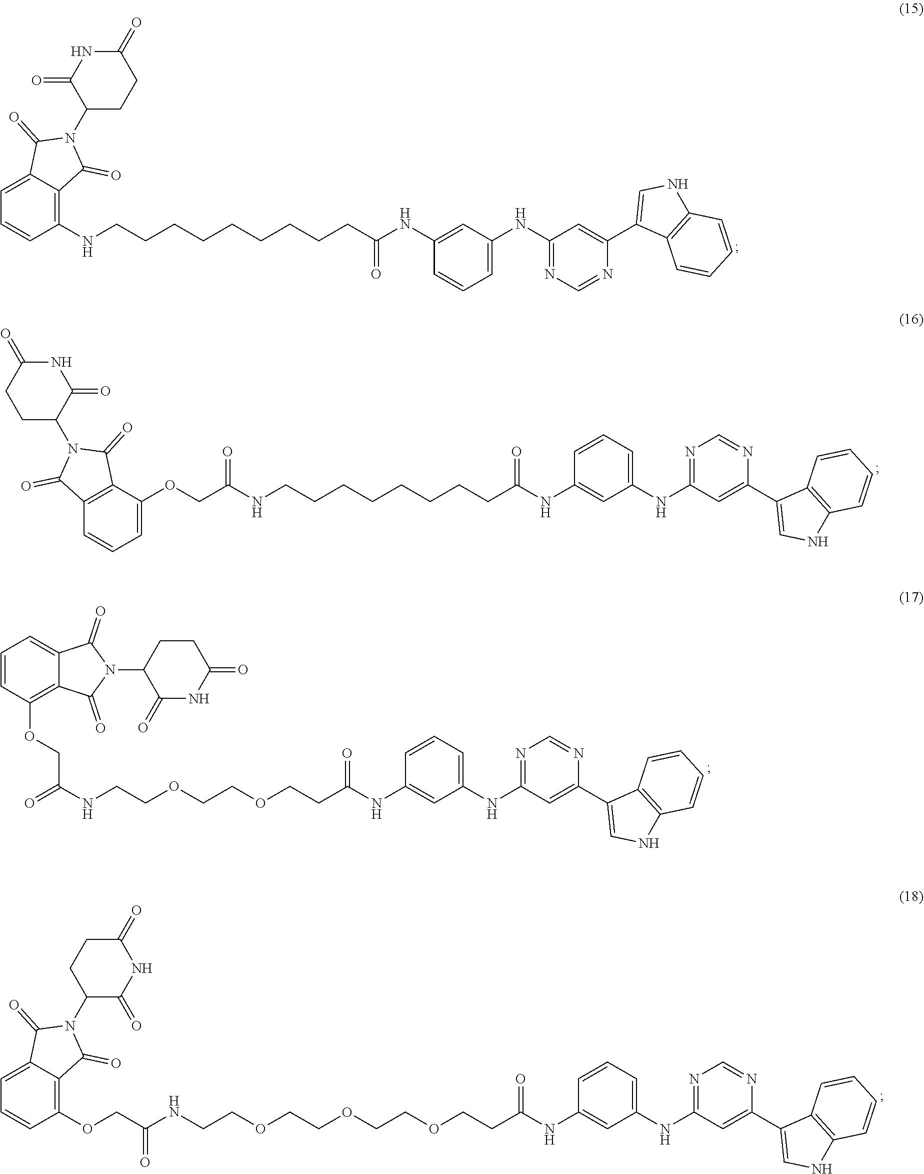 [SMILES: O=C1CCC(N2C(=O)c3cccc(NCCCCCCCCCC(=O)Nc4cccc(Nc5cc(-c6c[nH]c7ccccc67)ncn5)c4)c3C2=O)C(=O)N1 ; O=C(COc1cccc2c1C(=O)N(C1CCC(=O)NC1=O)C2=O)NCCCCCCCCC(=O)Nc1cccc(Nc2cc(-c3c[nH]c4ccccc34)ncn2)c1 ; O=C(COc1cccc2c1C(=O)N(C1CCC(=O)NC1=O)C2=O)NCCOCCOCCC(=O)Nc1cccc(Nc2cc(-c3c[nH]c4ccccc34)ncn2)c1 ; O=C(COc1cccc2c1C(=O)N(C1CCC(=O)NC1=O)C2=O)NCCOCCOCCOCCC(=O)Nc1cccc(Nc2cc(-c3c[nH]c4ccccc34)ncn2)c1] 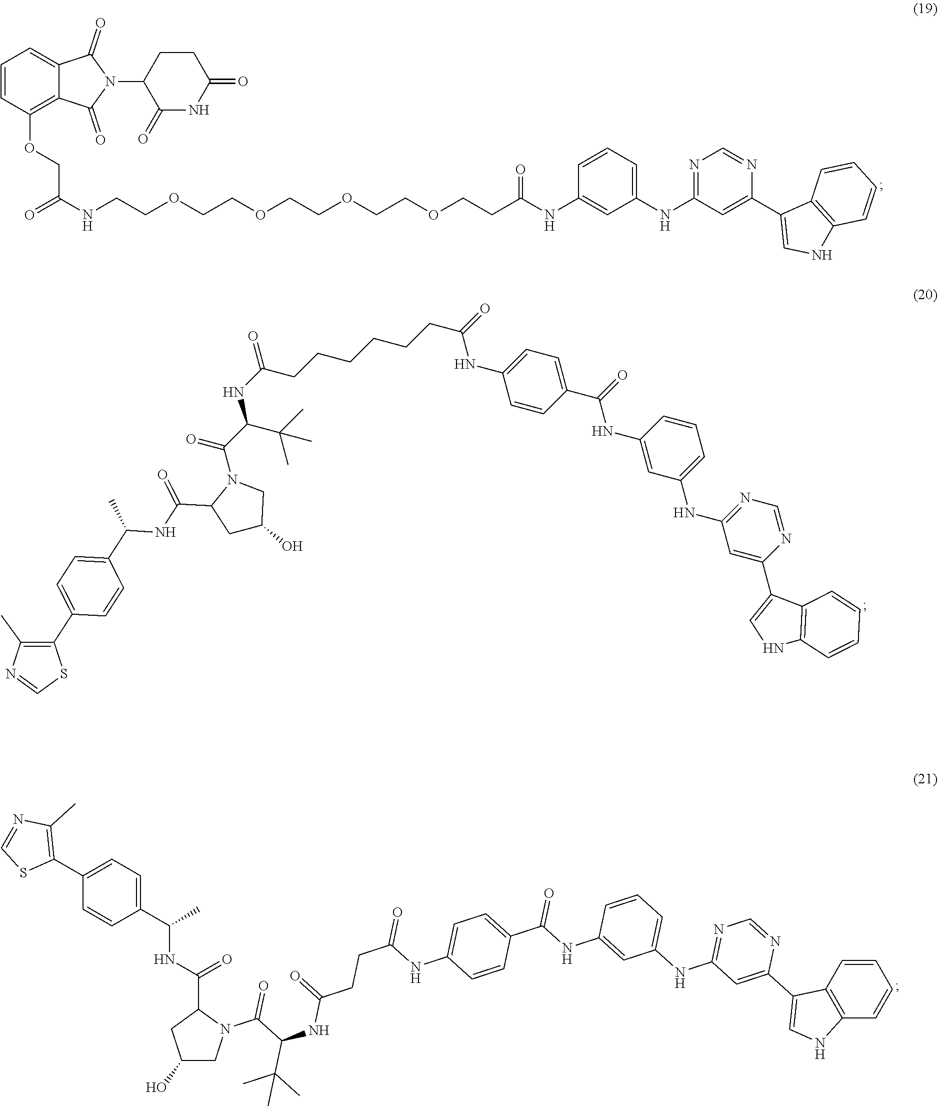 [SMILES: O=C(COc1cccc2c1C(=O)N(C1CCC(=O)NC1=O)C2=O)NCCOCCOCCOCCOCCC(=O)Nc1cccc(Nc2cc(-c3c[nH]c4ccccc34)ncn2)c1 ; Cc1ncsc1-c1ccc([C@H](C)NC(=O)C2C[C@@H](O)CN2C(=O)[C@@H](NC(=O)CCCCCCC(=O)Nc2ccc(C(=O)Nc3cccc(Nc4cc(-c5c[nH]c6ccccc56)ncn4)c3)cc2)C(C)(C)C)cc1 ; Cc1ncsc1-c1ccc([C@H](C)NC(=O)C2C[C@@H](O)CN2C(=O)[C@@H](NC(=O)CCC(=O)Nc2ccc(C(=O)Nc3cccc(Nc4cc(-c5c[nH]c6ccccc56)ncn4)c3)cc2)C(C)(C)C)cc1] 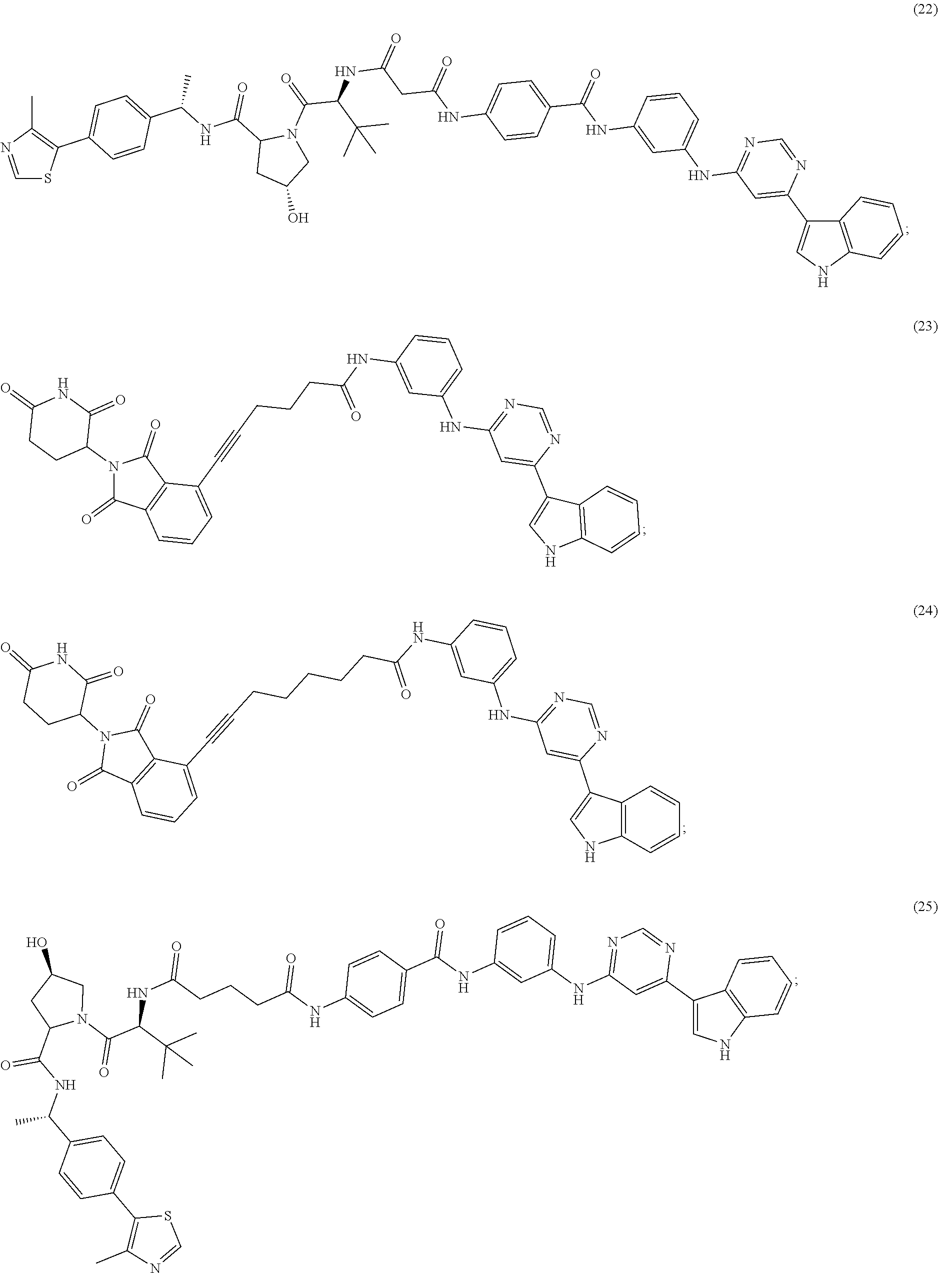 [SMILES: Cc1ncsc1-c1ccc([C@H](C)NC(=O)C2C[C@@H](O)CN2C(=O)[C@@H](NC(=O)CC(=O)Nc2ccc(C(=O)Nc3cccc(Nc4cc(-c5c[nH]c6ccccc56)ncn4)c3)cc2)C(C)(C)C)cc1 ; O=C1CCC(N2C(=O)c3cccc(C#CCCCC(=O)Nc4cccc(Nc5cc(-c6c[nH]c7ccccc67)ncn5)c4)c3C2=O)C(=O)N1 ; O=C1CCC(N2C(=O)c3cccc(C#CCCCCCC(=O)Nc4cccc(Nc5cc(-c6c[nH]c7ccccc67)ncn5)c4)c3C2=O)C(=O)N1 ; Cc1ncsc1-c1ccc([C@H](C)NC(=O)C2C[C@@H](O)CN2C(=O)[C@@H](NC(=O)CCCC(=O)Nc2ccc(C(=O)Nc3cccc(Nc4cc(-c5c[nH]c6ccccc56)ncn4)c3)cc2)C(C)(C)C)cc1] 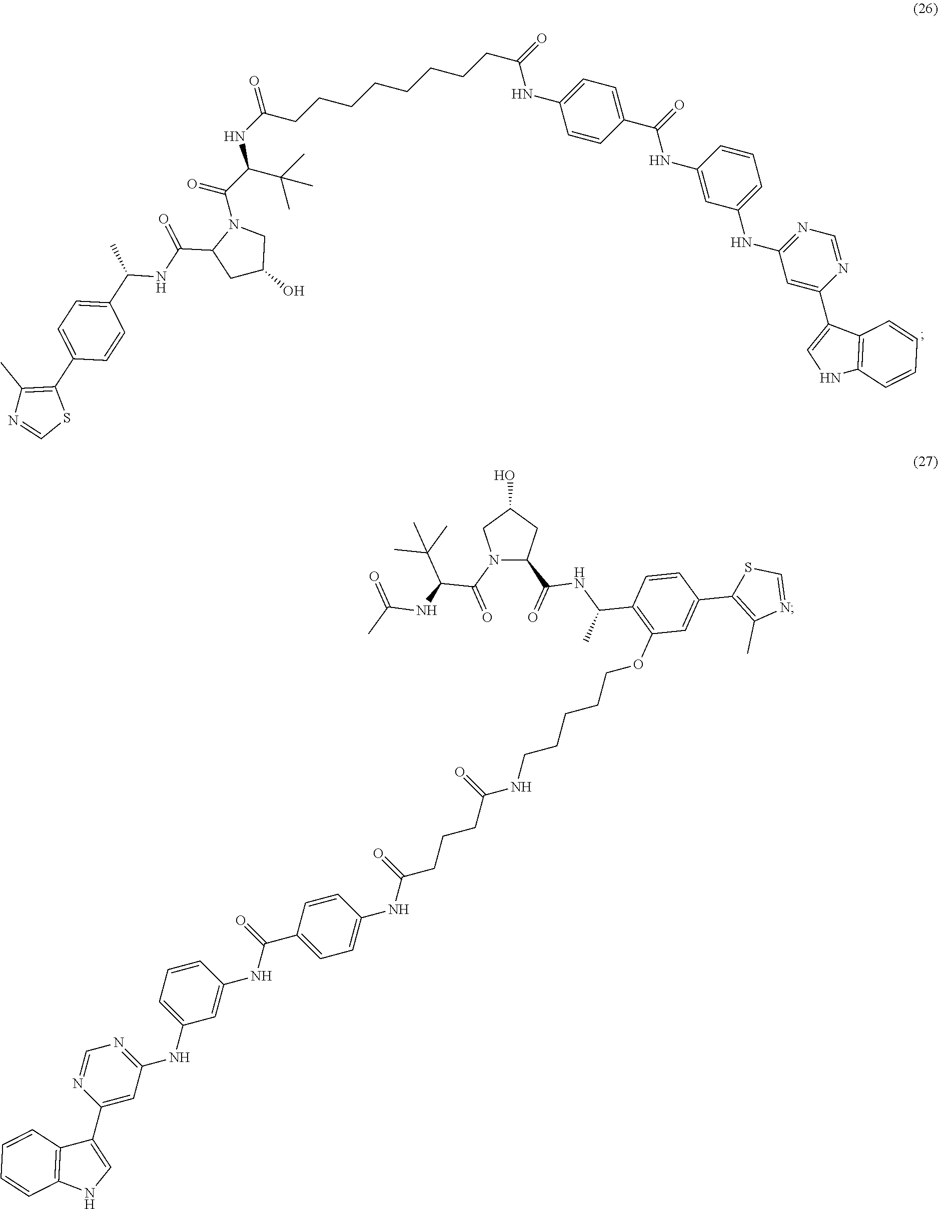 [SMILES: Cc1ncsc1-c1ccc([C@H](C)NC(=O)C2C[C@@H](O)CN2C(=O)[C@@H](NC(=O)CCCCCCCCC(=O)Nc2ccc(C(=O)Nc3cccc(Nc4cc(-c5c[nH]c6ccccc56)ncn4)c3)cc2)C(C)(C)C)cc1 ; CC(=O)N[C@H](C(=O)N1C[C@H](O)C[C@H]1C(=O)N[C@@H](C)c1ccc(-c2scnc2C)cc1OCCCCCNC(=O)CCCC(=O)Nc1ccc(C(=O)Nc2cccc(Nc3cc(-c4c[nH]c5ccccc45)ncn3)c2)cc1)C(C)(C)C] 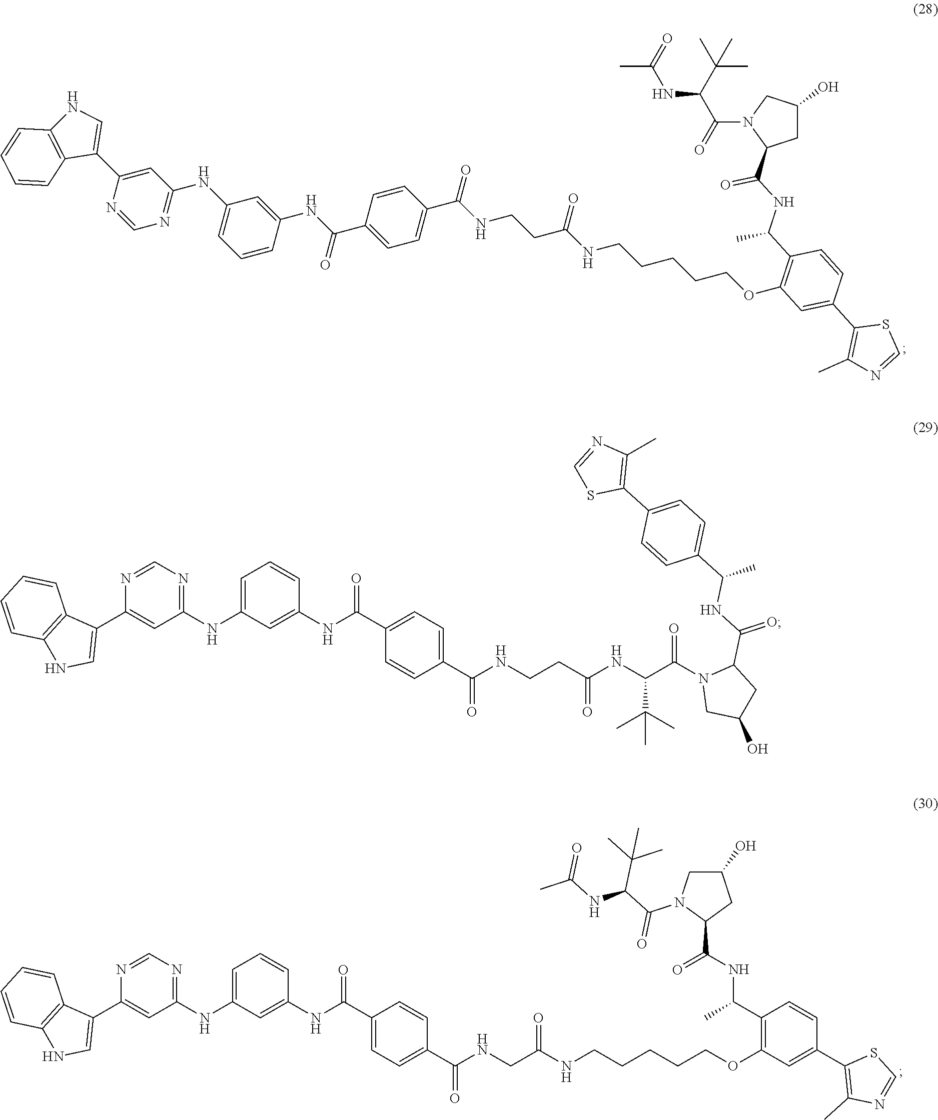 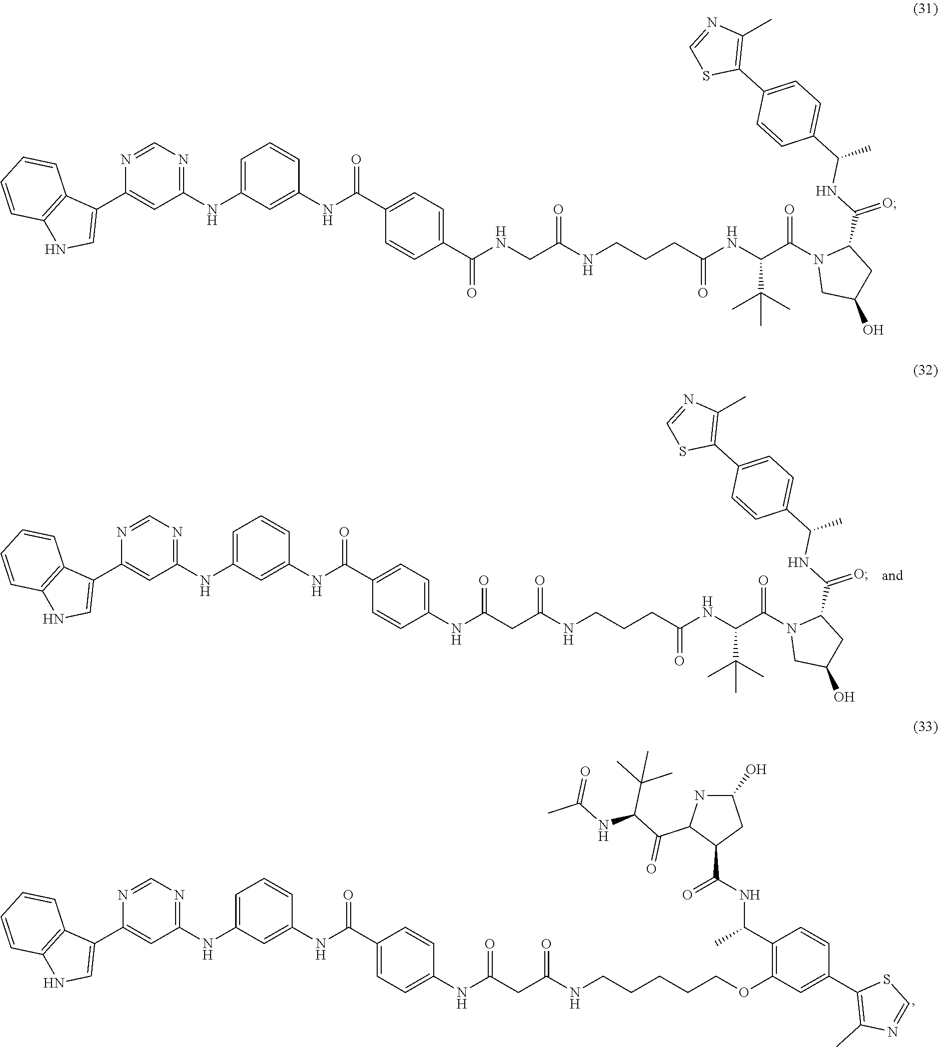
and pharmaceutically acceptable salts and stereoisomers thereof.

33. The bifunctional compound of claim 1, which represented by any one of structures 1, 8, and 14: 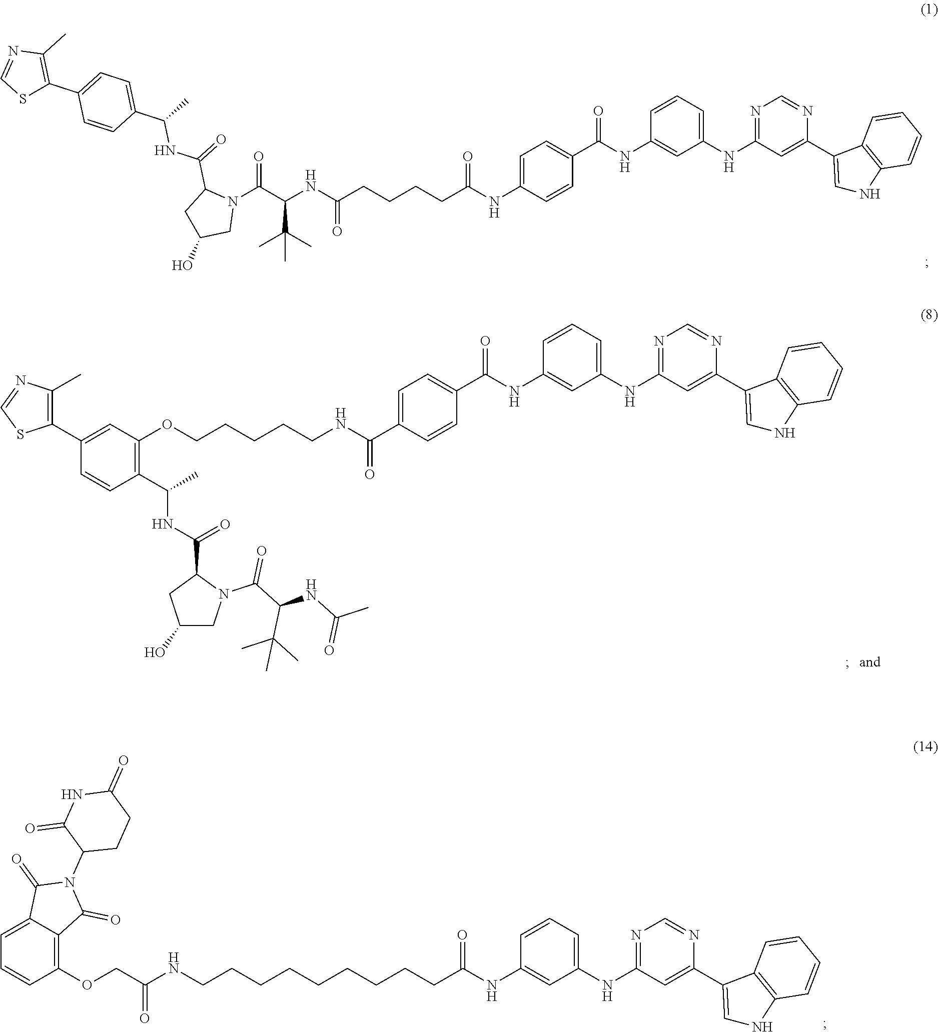 [SMILES: Cc1ncsc1-c1ccc([C@H](C)NC(=O)C2C[C@@H](O)CN2C(=O)[C@@H](NC(=O)CCCCC(=O)Nc2ccc(C(=O)Nc3cccc(Nc4cc(-c5c[nH]c6ccccc56)ncn4)c3)cc2)C(C)(C)C)cc1 ; CC(=O)N[C@H](C(=O)N1C[C@H](O)C[C@H]1C(=O)N[C@@H](C)c1ccc(-c2scnc2C)cc1OCCCCCNC(=O)c1ccc(C(=O)Nc2cccc(Nc3cc(-c4c[nH]c5ccccc45)ncn3)c2)cc1)C(C)(C)C ; O=C(COc1cccc2c1C(=O)N(C1CCC(=O)NC1=O)C2=O)NCCCCCCCCCC(=O)Nc1cccc(Nc2cc(-c3c[nH]c4ccccc34)ncn2)c1]
or a pharmaceutically acceptable salt or stereoisomer thereof.

34. A pharmaceutical composition, comprising a therapeutically effective amount of the bifunctional compound or pharmaceutically acceptable salt or stereoisomer thereof of claim 1, and a pharmaceutically acceptable carrier.

35. A method of treating a disease or disorder by modulating the level or activity of at least one of PIP4K2A, PIP4K2B, and PIP4K2C, comprising administering to a subject in need thereof a therapeutically effective amount of the bifunctional compound or pharmaceutically acceptable salt or stereoisomer thereof of claim 1.

36. (canceled)

37. The method of claim 35, wherein the disease or disorder is a cancer, immune deficiency, autoimmune disease, or infectious disease, or a combination thereof, or a neurodegenerative disease.

38. The method of claim 37, wherein the cancer is leukemia, lymphoma or multiple myeloma.

39. (canceled)

40. The method of claim 39, wherein the disease or disorder is insulin resistance or Huntington's disease.

41. (canceled)

42. (canceled)

43. A method of modulating immune functions in a subject in need thereof, comprising administering to a subject a therapeutically effective amount of the bifunctional compound or pharmaceutically acceptable salt or stereoisomer thereof of claim 1.

44. A method of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of the bifunctional compound or pharmaceutically acceptable salt or stereoisomer thereof of claim 1.
